(19) United States
(12) Patent Application Publication
(10) Pub. No.: US 2018/0078276 A1
(43) Pub. Date: Mar. 22, 2018

(54) Concentric Cutting Devices for Use in Minimally Invasive Medical Procedures

(71) Applicant: Microfabrica Inc., Van Nuys, CA (US)

(72) Inventors: Richard T. Chen, Stevenson Ranch, CA (US); Ming Ting Wu, San Jose, CA (US); Arun S. Veeramani, Vista, CA (US); Vacit Arat, Pasadena, CA (US); Gregory P. Schmitz, Los Gatos, CA (US); Juan Diego Perea, Campbell, CA (US); Ronald Leguidleguid, Union City, CA (US); Gregory B. Arcenio, Redwood City, CA (US); Eric C. Miller, Los Gatos, CA (US)

(73) Assignee: Microfabrica Inc., Van Nuys, CA (US)

(21) Appl. No.: 15/718,780

(22) Filed: Sep. 28, 2017

Related U.S. Application Data
(63) Continuation-in-part of application No. 14/181,247, filed on Feb. 14, 2014, now abandoned.
Application No. 14/181,247 is a continuation of application No. 13/388,653, filed on Apr. 16, 2012, now abandoned.
Application No. 13/388,653 is a continuation-in-part of application No. 13/714,285, filed on Dec. 13, 2012, now Pat. No. 9,814,484.
Application No. 13/714,285 is a continuation-in-part of application No. 14/033,397, filed on Sep. 20, 2013, now abandoned.
Application No. 14/033,397 is a continuation-in-part of application No. 14/440,088, filed on May 1, 2015.
Application No. 14/440,088 is a continuation-in-part of application No. 15/292,029, filed on Oct. 12, 2016.
Application No. 15/292,029 is a continuation-in-part of application No. 13/855,627, filed on Apr. 2, 2013, now abandoned.
(60) Provisional application No. 61/234,989, filed on Aug. 18, 2009.
(60) Provisional application No. 61/731,434, filed on Nov. 29, 2012.
(60) Provisional application No. 61/731,091, filed on Nov. 29, 2012.
(60) Provisional application No. 61/728,443, filed on Nov. 20, 2012.
(60) Provisional application No. 61/710,608, filed on Oct. 5, 2012.
(60) Provisional application No. 62/385,829, filed on Sep. 9, 2016.

(51) Int. Cl. A61B 17/3205

(52) U.S. Cl. CPC A61B 17/3205; A61B 2017/320064

(57) ABSTRACT

Various embodiments of a tissue cutting device and methods for using are described. In some variations devices include an elongate tube having a proximal end and a distal end and a central axis extending from the proximal end to the distal end; a first annular element at the distal end of the elongate tube, the first annular element having a cutting portion at its distal; and a second annular element at the distal end of the elongate tube and concentric with the first annular element, the second annular element having a cutting portion at its distal end, the first and second annular elements being rotatable relative to one another to cause the first annular element and the second annular element to pass each other to shear tissue.

### RELATED APPLICATIONS

The below table sets forth the priority claims for the instant application along with filing dates, patent numbers, and issue dates as appropriate. Each of the listed applications is incorporated herein by reference as if set forth in full herein including any appendices attached thereto.

| | | | Which was | | | |
|---|---|---|---|---|---|---|
| | | | Filed | | | |
| | Continuity | | (YYYY-MM- | Which is | Which | Dkt No. |
| App. No. | Type | App. No. | DD) | now | issued on | Fragment |
| This | is a CIP of | 14/181,247 | 2014-02-14 | pending | — | US295-B |
| application | | | | | | |
| 14/181,247 | is a CNT of | 13/388,653 | 2012-04-16 | pending | — | US295-A |
| 13/388,653 | is a 371 of | PCT/ | 2010-08-18 | expired | — | WO295-A |
| | | US2010/ | | | | |
| | | 045951 | | | | |
| PCT/ | claims | 61/234,989 | 2009-08-18 | expired | — | US269-A |
| US2010/ | benefit of | | | | | |
| 045951 | | | | | | |
| This | is a CIP of | 13/714,285 | 2012-12-13 | pending | — | US309-A |
| application | | | | | | |
| 13/714,285 | claims | 61/731,434 | 2012-11-29 | expired | — | US307-A |
| | benefit of | | | | | |
| This | is a CIP of | 14/033,397 | 2013-09-20 | pending | — | US318-A |
| application | | | | | | |
| 14/033,397 | claims | 61/731,091 | 2012-11-29 | expired | — | US320-A |
| | benefit of | | | | | |
| This | is a CIP of | 14/440,088 | 2015-05-01 | pending | — | US323-A |
| application | | | | | | |
| 14/440,088 | is a 371 of | PCT/ | 2013-11-20 | expired | — | WO323-A |
| | | US2013/ | | | | |
| | | 070909 | | | | |
| PCT/ | claims | 61/728,443 | 2012-11-20 | expired | — | US319-A |
| US2013/ | benefit of | | | | | |
| 070909 | | | | | | |
| This | is a CIP of | 15/292,029 | 2016-10-12 | pending | — | US351-A |
| application | | | | | | |
| 15/292,029 | is a CIP of | 13/855,627 | 2013-04-02 | abandoned | — | US312-A |
| 13/855,627 | claims | 61/710,608 | 2012-10-05 | expired | — | US304-A |
| | benefit of | | | | | |
| 15/292,029 | claims | 62/385,829 | 2016-09-09 | expired | — | US348-A |
| | benefit of | | | | | |

### U.S. GOVERNMENT RIGHTS

This invention was made with government support under Grant No. R01 HL087797 awarded by the National Institutes of Health. The government has certain rights in the invention.

### FIELD OF THE INVENTION

Embodiments of the present invention relate to micro-scale and millimeter-scale cutting devices that may be located at the distal ends of, or at intermediate positions along the length of, a lumen to provide material cutting, shredding, and removal. Such devices may, for example, be used to remove unwanted tissue or other material from selected locations within a body of a patient during minimally invasive or other medical procedures. In some embodiments, such devices may be used for non-medical procedure and in some embodiments the devices may be made in whole or in part using multi-layer, multi-material fabrication methods such as electrochemical fabrication methods.

### BACKGROUND OF THE INVENTION

Electrochemical Fabrication:

An electrochemical fabrication technique for forming three-dimensional structures from a plurality of adhered layers is being commercially pursued by Microfabrica® Inc. (formerly MEMGen Corporation) of Van Nuys, Calif. under the process names EFAB™ and MICA FREEFORM®.

Various electrochemical fabrication techniques were described in U.S. Pat. No. 6,027,630, issued on Feb. 22, 2000 to Adam Cohen. Some embodiments of this electrochemical fabrication technique allows the selective deposition of a material using a mask that includes a patterned conformable material on a support structure that is independent of the substrate onto which plating will occur. When desiring to perform an electrodeposition using the mask, the conformable portion of the mask is brought into contact with a substrate, but not adhered or bonded to the substrate, while in the presence of a plating solution such that the contact of the conformable portion of the mask to the substrate inhibits deposition at selected locations. For convenience, these masks might be generically called conformable contact masks; the masking technique may be generically called a conformable contact mask plating process. More specifically, in the terminology of Microfabrica Inc. such masks have come to be known as INSTANT MASKS™ and the process known as INSTANT MASKING™ or INSTANT MASK™ plating. Selective depositions using conformable contact mask plating may be used to form single selective deposits of material or may be used in a process to form multi-layer structures. The teachings of the '630 patent are hereby incorporated herein by reference as if set forth in full herein. Since the filing of the patent application that led to the above noted patent, various papers about conformable contact mask plating (i.e. INSTANT MASKING) and electrochemical fabrication have been published:
(1) A. Cohen, G. Zhang, F. Tseng, F. Mansfeld, U. Frodis and P. Will, “EFAB: Batch production of functional, fully-dense metal parts with micro-scale features”, Proc. 9th Solid Freeform Fabrication, The University of Texas at Austin, p 161, August 1998.
(2) A. Cohen, G. Zhang, F. Tseng, F. Mansfeld, U. Frodis and P. Will, “EFAB: Rapid, Low-Cost Desktop Micromachining of High Aspect Ratio True 3-D MEMS”, Proc. 12th IEEE Micro Electro Mechanical Systems Workshop, IEEE, p 244, January 1999.
(3) A. Cohen, “3-D Micromachining by Electrochemical Fabrication”, Micromachine devices, March 1999.
(4) G. Zhang, A. Cohen, U. Frodis, F. Tseng, F. Mansfeld, and P. Will, “EFAB: Rapid Desktop Manufacturing of True 3-D Microstructures”, Proc. 2nd International Conference on Integrated MicroNanotechnology for Space Applications, The Aerospace Co., April 1999.
(5) F. Tseng, U. Frodis, G. Zhang, A. Cohen, F. Mansfeld, and P. Will, “EFAB: High Aspect Ratio, Arbitrary 3-D Metal Microstructures using a Low-Cost Automated Batch Process”, 3rd International Workshop on High Aspect Ratio MicroStructure Technology (HARMST'99), June 1999.
(6) A. Cohen, U. Frodis, F. Tseng, G. Zhang, F. Mansfeld, and P. Will, “EFAB: Low-Cost, Automated Electrochemical Batch Fabrication of Arbitrary 3-D Microstructures”, Micromachining and Microfabrication Process Technology, SPIE 1999 Symposium on Micromachining and Microfabrication, September 1999.
(7) F. Tseng, G. Zhang, U. Frodis, A. Cohen, F. Mansfeld, and P. Will, “EFAB: High Aspect Ratio, Arbitrary 3-D Metal Microstructures using a Low-Cost Automated Batch Process”, MEMS Symposium, ASME 1999 International Mechanical Engineering Congress and Exposition, November, 1999.
(8) A. Cohen, “Electrochemical Fabrication (EFAB™)”, Chapter 19 of The MEMS Handbook, edited by Mohamed Gad-El-Hak, CRC Press, 2002.
(9) Microfabrication—Rapid Prototyping's Killer Application”, pages 1-5 of the Rapid Prototyping Report, CAD/CAM Publishing, Inc., June 1999.

The disclosures of these nine publications are hereby incorporated herein by reference as if set forth in full herein.

An electrochemical deposition for forming multilayer structures may be carried out in a number of different ways as set forth in the above patent and publications. In one form, this process involves the execution of three separate operations during the formation of each layer of the structure that is to be formed:

1. Selectively depositing at least one material by electrodeposition upon one or more desired regions of a substrate. Typically this material is either a structural material or a sacrificial material.

2. Then, blanket depositing at least one additional material by electrodeposition so that the additional deposit covers both the regions that were previously selectively deposited onto, and the regions of the substrate that did not receive any previously applied selective depositions. Typically this material is the other of a structural material or a sacrificial material.

3. Finally, planarizing the materials deposited during the first and second operations to produce a smoothed surface of a first layer of desired thickness having at least one region containing the at least one material and at least one region containing at least the one additional material.

After formation of the first layer, one or more additional layers may be formed adjacent to an immediately preceding layer and adhered to the smoothed surface of that preceding layer. These additional layers are formed by repeating the first through third operations one or more times wherein the formation of each subsequent layer treats the previously formed layers and the initial substrate as a new and thickening substrate.

Once the formation of all layers has been completed, at least a portion of at least one of the materials deposited is generally removed by an etching process to expose or release the three-dimensional structure that was intended to be formed. The removed material is a sacrificial material while the material that forms part of the desired structure is a structural material.

The preferred method of performing the selective electrodeposition involved in the first operation is by conformable contact mask plating. In this type of plating, one or more conformable contact (CC) masks are first formed. The CC masks include a support structure onto which a patterned conformable dielectric material is adhered or formed. The conformable material for each mask is shaped in accordance with a particular cross-section of material to be plated (the pattern of conformable material is complementary to the pattern of material to be deposited). At least one CC mask is used for each unique cross-sectional pattern that is to be plated.

The support for a CC mask is typically a plate-like structure formed of a metal that is to be selectively electroplated and from which material to be plated will be dissolved. In this typical approach, the support will act as an anode in an electroplating process. In an alternative approach, the support may instead be a porous or otherwise perforated material through which deposition material will pass during an electroplating operation on its way from a distal anode to a deposition surface. In either approach, it is possible for multiple CC masks to share a common support, i.e. the patterns of conformable dielectric material for plating multiple layers of material may be located in different areas of a single support structure. When a single support structure contains multiple plating patterns, the entire structure is referred to as the CC mask while the individual plating masks may be referred to as “submasks”. In the present application such a distinction will be made only when relevant to a specific point being made.

In preparation for performing the selective deposition of the first operation, the conformable portion of the CC mask is placed in registration with and pressed against a selected portion of (1) the substrate, (2) a previously formed layer, or (3) a previously deposited portion of a layer on which deposition is to occur. The pressing together of the CC mask and relevant substrate occur in such a way that all openings, in the conformable portions of the CC mask contain plating solution. The conformable material of the CC mask that contacts the substrate acts as a barrier to electrodeposition while the openings in the CC mask that are filled with electroplating solution act as pathways for transferring material from an anode (e.g. the CC mask support) to the non-contacted portions of the substrate (which act as a cathode during the plating operation) when an appropriate potential and/or current are supplied.

An example of a CC mask and CC mask plating are shown in FIGS. 1A-1C. FIG. 1A shows a side view of a CC mask 8 consisting of a conformable or deformable (e.g. elastomeric) insulator 10 patterned on an anode 12. The anode has two functions. One is as a supporting material for the patterned insulator 10 to maintain its integrity and alignment since the pattern may be topologically complex (e.g., involving isolated “islands” of insulator material). The other function is as an anode for the electroplating operation. FIG. 1A also depicts a substrate 6, separated from mask 8, onto which material will be deposited during the process of forming a layer. CC mask plating selectively deposits material 22 onto substrate 6 by simply pressing the insulator against the substrate then electrodepositing material through apertures 26a and 26b in the insulator as shown in FIG. 1B. After deposition, the CC mask is separated, preferably non-destructively, from the substrate 6 as shown in FIG. 1C.

The CC mask plating process is distinct from a “through-mask” plating process in that in a through-mask plating process the separation of the masking material from the substrate would occur destructively. Furthermore in a through mask plating process, opening in the masking material are typically formed while the masking material is in contact with and adhered to the substrate. As with through-mask plating, CC mask plating deposits material selectively and simultaneously over the entire layer. The plated region may consist of one or more isolated plating regions where these isolated plating regions may belong to a single structure that is being formed or may belong to multiple structures that are being formed simultaneously. In CC mask plating as individual masks are not intentionally destroyed in the removal process, they may be usable in multiple plating operations.

Another example of a CC mask and CC mask plating is shown in FIGS. 1D-1G. FIG. 1D shows an anode 12′ separated from a mask 8′ that includes a patterned conformable material 10′ and a support structure 20. FIG. 1D also depicts substrate 6 separated from the mask 8′. FIG. 1E illustrates the mask 8′ being brought into contact with the substrate 6. FIG. 1F illustrates the deposit 22′ that results from conducting a current from the anode 12′ to the substrate 6. FIG. 1G illustrates the deposit 22′ on substrate 6 after separation from mask 8′. In this example, an appropriate electrolyte is located between the substrate 6 and the anode 12′ and a current of ions coming from one or both of the solution and the anode are conducted through the opening in the mask to the substrate where material is deposited. This type of mask may be referred to as an anodeless INSTANT MASK™ (AIM) or as an anodeless conformable contact (ACC) mask.

Unlike through-mask plating, CC mask plating allows CC masks to be formed completely separate from the substrate on which plating is to occur (e.g. separate from a three-dimensional (3D) structure that is being formed). CC masks may be formed in a variety of ways, for example, using a photolithographic process. All masks can be generated simultaneously, e.g. prior to structure fabrication rather than during it. This separation makes possible a simple, low-cost, automated, self-contained, and internally-clean “desktop factory” that can be installed almost anywhere to fabricate 3D structures, leaving any required clean room processes, such as photolithography to be performed by service bureaus or the like.

An example of the electrochemical fabrication process discussed above is illustrated in FIGS. 2A-2F. These figures show that the process involves deposition of a first material 2 which is a sacrificial material and a second material 4 which is a structural material. The CC mask 8, in this example, includes a patterned conformable material (e.g. an elastomeric dielectric material) 10 and a support 12 which is made from deposition material 2. The conformal portion of the CC mask is pressed against substrate 6 with a plating solution 14 located within the openings 16 in the conformable material 10. An electric current, from power supply 18, is then passed through the plating solution 14 via (a) support 12 which doubles as an anode and (b) substrate 6 which doubles as a cathode. FIG. 2A illustrates that the passing of current causes material 2 within the plating solution and material 2 from the anode 12 to be selectively transferred to and plated on the substrate 6. After electroplating the first deposition material 2 onto the substrate 6 using CC mask 8, the CC mask 8 is removed as shown in FIG. 2B. FIG. 2C depicts the second deposition material 4 as having been blanket-deposited (i.e. non-selectively deposited) over the previously deposited first deposition material 2 as well as over the other portions of the substrate 6. The blanket deposition occurs by electroplating from an anode (not shown), composed of the second material, through an appropriate plating solution (not shown), and to the cathode/substrate 6. The entire two-material layer is then planarized to achieve precise thickness and flatness as shown in FIG. 2D. After repetition of this process for all layers, the multi-layer structure 20 formed of the second material 4 (i.e. structural material) is embedded in first material 2 (i.e. sacrificial material) as shown in FIG. 2E. The embedded structure is etched to yield the desired device, i.e. structure 20, as shown in FIG. 2F.

Various components of an exemplary manual electrochemical fabrication system 32 are shown in FIGS. 3A-3C. The system 32 consists of several subsystems 34, 36, 38, and 40. The substrate holding subsystem 34 is depicted in the upper portions of each of FIGS. 3A-3C and includes several components: (1) a carrier 48, (2) a metal substrate 6 onto which the layers are deposited, and (3) a linear slide 42 capable of moving the substrate 6 up and down relative to the carrier 48 in response to drive force from actuator 44. Subsystem 34 also includes an indicator 46 for measuring differences in vertical position of the substrate which may be used in setting or determining layer thicknesses and/or deposition thicknesses. The subsystem 34 further includes feet 68 for carrier 48 which can be precisely mounted on subsystem 36.

The CC mask subsystem 36 shown in the lower portion of FIG. 3A includes several components: (1) a CC mask 8 that is actually made up of a number of CC masks (i.e. submasks) that share a common support/anode 12, (2) precision X-stage 54, (3) precision Y-stage 56, (4) frame 72 on which the feet 68 of subsystem 34 can mount, and (5) a tank 58 for containing the electrolyte 16. Subsystems 34 and 36 also include appropriate electrical connections (not shown) for connecting to an appropriate power source (not shown) for driving the CC masking process. The blanket deposition subsystem 38 is shown in the lower portion of FIG. 3B and includes several components: (1) an anode 62, (2) an electrolyte tank 64 for holding plating solution 66, and (3) frame 74 on which feet 68 of subsystem 34 may sit. Subsystem 38 also includes appropriate electrical connections (not shown) for connecting the anode to an appropriate power supply (not shown) for driving the blanket deposition process.

The planarization subsystem 40 is shown in the lower portion of FIG. 3C and includes a lapping plate 52 and associated motion and control systems (not shown) for planarizing the depositions.

In addition to teaching the use of CC masks for electrodeposition purposes, the '630 patent also teaches that the CC masks may be placed against a substrate with the polarity of the voltage reversed and material may thereby be selectively removed from the substrate. It indicates that such removal processes can be used to selectively etch, engrave, and polish a substrate, e.g., a plaque.

The '630 patent further indicates that the electroplating methods and articles disclosed therein allow fabrication of devices from thin layers of materials such as, e.g., metals, polymers, ceramics, and semiconductor materials. It further indicates that although the electroplating embodiments described therein have been described with respect to the use of two metals, a variety of materials, e.g., polymers, ceramics and semiconductor materials, and any number of metals can be deposited either by the electroplating methods therein, or in separate processes that occur throughout the electroplating method. It indicates that a thin plating base can be deposited, e.g., by sputtering, over a deposit that is insufficiently conductive (e.g., an insulating layer) so as to enable subsequent electroplating. It also indicates that multiple support materials (i.e. sacrificial materials) can be included in the electroplated element allowing selective removal of the support materials.

The '630 patent additionally teaches that the electroplating methods disclosed therein can be used to manufacture elements having complex microstructure and close tolerances between parts. An example is given with the aid of FIGS. 14A-14E of that patent. In the example, elements having parts that fit with close tolerances, e.g., having gaps between about 1-5 um, including electroplating the parts of the device in an unassembled, preferably pre-aligned, state and once fabricated. In such embodiments, the individual parts can be moved into operational relation with each other or they can simply fall together. Once together the separate parts may be retained by clips or the like.

Another method for forming microstructures from electroplated metals (i.e. using electrochemical fabrication techniques) is taught in U.S. Pat. No. 5,190,637 to Henry Guckel, entitled “Formation of Microstructures by Multiple Level Deep X-ray Lithography with Sacrificial Metal layers”. This patent teaches the formation of metal structure utilizing through mask exposures. A first layer of a primary metal is electroplated onto an exposed plating base to fill a void in a photoresist (the photoresist forming a through mask having a desired pattern of openings), the photoresist is then removed and a secondary metal is electroplated over the first layer and over the plating base. The exposed surface of the secondary metal is then machined down to a height which exposes the first metal to produce a flat uniform surface extending across both the primary and secondary metals. Formation of a second layer may then begin by applying a photoresist over the first layer and patterning it (i.e. to form a second through mask) and then repeating the process that was used to produce the first layer to produce a second layer of desired configuration. The process is repeated until the entire structure is formed and the secondary metal is removed by etching. The photoresist is formed over the plating base or previous layer by casting and patterning of the photoresist (i.e. voids formed in the photoresist) are formed by exposure of the photoresist through a patterned mask via X-rays or UV radiation and development of the exposed or unexposed areas.

The '637 patent teaches the locating of a plating base onto a substrate in preparation for electroplating materials onto the substrate. The plating base is indicated as typically involving the use of a sputtered film of an adhesive metal, such as chromium or titanium, and then a sputtered film of the metal that is to be plated. It is also taught that the plating base may be applied over an initial layer of sacrificial material (i.e. a layer or coating of a single material) on the substrate so that the structure and substrate may be detached if desired. In such cases after formation of the structure the sacrificial material forming part of each layer of the structure may be removed along the initial sacrificial layer to free the structure. Substrate materials mentioned in the '637 patent include silicon, glass, metals, and silicon with protected semiconductor devices. A specific example of a plating base includes about 150 angstroms of titanium and about 300 angstroms of nickel, both of which are sputtered at a temperature of 160° C. In another example it is indicated that the plating base may consist of 150 angstroms of titanium and 150 angstroms of nickel where both are applied by sputtering.

Electrochemical Fabrication provides the ability to form prototypes and commercial quantities of miniature objects, parts, structures, devices, and the like at reasonable costs and in reasonable times. In fact, Electrochemical Fabrication is an enabler for the formation of many structures that were hitherto impossible to produce. Electrochemical Fabrication opens the spectrum for new designs and products in many industrial fields. Even though Electrochemical Fabrication offers this new capability and it is understood that Electrochemical Fabrication techniques can be combined with designs and structures known within various fields to produce new structures, certain uses for Electrochemical Fabrication provide designs, structures, capabilities and/or features not known or obvious in view of the state of the art.

A need exists in various fields for miniature devices having improved characteristics, reduced fabrication times, reduced fabrication costs, simplified fabrication processes, greater versatility in device design, improved selection of materials, improved material properties, more cost effective and less risky production of such devices, and/or more independence between geometric configuration and the selected fabrication process.

Material Removal Devices for Medical Applications

Various mechanical material breakdown and/or removal methods and devices have been proposed and/or used in minimally invasive medical applications such as thrombectomy and atherectomy procedures. These devices can be used in medical procedures including planning, coring, milling, and drilling. Such devices, for example, have included the use of cutting elements, shaving elements, and grinding elements. Examples of cutting devices are found, for example in (1) US Patent Application Publication No. 2006/0212060 A1, entitled “Arthroscopic Shaver and Method of Manufacturing Same” by Randall L. Hacker, et al. and assigned to Arthex, Inc.; (2) U.S. Pat. No. 6,447,525; (3) U.S. Pat. No. 7,479,147; and (4) U.S. Pat. No. 7,235,088.

Planing devices can be used to surface thin layers of tissue, e.g. for removing scars from the surface of the skin. Conventional planing devices include at least one sharp edge that can be translated across the tissue to remove the top-most layer. Such cutting surfaces in conventional planing devices generally have dimensions that are too large to cut thin slices of tissue, e.g. to cut slices of tissue having a thickness less than 50 μm, and these devices therefore cannot precisely remove small areas of tissue.

Coring devices can be used for biopsying tissue. Conventional coring devices generally include a needle that bores into the tissue. Conventional coring devices tend to cause pulling of and damage to surrounding tissue as the needle is pushed in. The rapid forward movement of the needle can also push aside the target tissue, such as a suspected tumor, especially if the target tissue is firmer than the surrounding tissue. Further, conventional coring devices do not have small enough feature sizes to remove only small tissue particles, again resulting in excessive damage to surrounding tissue.

Milling devices, such as debriders, can be used for de-bulking, e.g. for surgical removal of a malignant tumor. Conventional debriders include a rounded or pointed distal end to aid in removing specific tissue. However, such conventional milling devices are disadvantageous in that they often remove too much tissue and, due to their rounded ends, cannot selectively remove surface tissue. Further, conventional milling devices have dimensions that are generally too large to precisely remove small areas of tissue.

Drilling devices, such as atherectomy devices, are used to cut through tissue in the body. For example, atherectomy devices are used to treat atherosclerosis, in which the arteries are obstructed due to the accumulation of plaque and neointimal hyperplasia. Such atherectomy devices work by cutting away or excising the obstructing plaque to help restore blood flow. Drilling devices are configured in a variety of ways, but generally include employing a rotatable and/or axially translatable cutting blade or abrasive end which can be advanced into the occluding material and rotated or translated to cut away the desired material. Conventional drilling devices, however, have several drawbacks. Namely, the minimum feature size and shape of such devices, e.g. the size and shape of the cutting blades, are often too large to cut specifically and precisely, such as down to a micrometer or cellular scale. As a result, such devices tend to either leave unwanted tissue in the body, such as plaque in the blood vessel, or cut too much tissue, thereby injuring surrounding tissue. Further, traditional drilling devices have a fairly large diameter, e.g. over 2 mm, and are not configured to fit into small lumens, such as blood vessels, having a smaller diameter. As a result, some areas in the body are unreachable by conventional drilling devices.

Accordingly, there is a need for small tissue-cutting devices, such as planing, coring, milling, or drilling devices that can precisely cut tissue down to a micrometer or cellular scale.

### SUMMARY OF THE INVENTION

This application and its parent applications are directed to, intra alia, miniature cylindrical devices for cutting tissue, systems that include such devices, methods for making such devices and/or systems, and medical procedures that use such devices or systems to provide a benefit to a patient (e.g. as part of a minimally invasive surgical procedure). As noted above teachings set forth in the parent applications are incorporated herein by reference and form an integral part of the teachings hereof. It is an object of some embodiments of the invention to provide an improved method for forming multi-layer three-dimensional structures.

It is an object of some embodiments of the invention to provide improved millimeter-scale or micro-scale devices that may be used in minimally invasive procedure to provide therapeutic, diagnostic, or preventive treatment.

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively may address some other object ascertained from the teachings herein. It is not necessarily intended that all objects be addressed by any single aspect of the invention even though that may be the case with regard to some aspects.

This application and its parent applications are directed to, intra alia, miniature cylindrical devices for cutting tissue, systems that include such devices, methods for making such devices and/or systems, and medical procedures that use such devices or systems to provide a benefit to a patient (e.g. as part of a minimally invasive surgical procedure). As noted above teachings set forth in the parent applications are incorporated herein by reference and form an integral part of the teachings hereof.

The Ser. No. 14/181,247 Application

This referenced application includes, inter alia, teachings directed to tissue cutting devices, such as devices with an elongate tubes having proximal ends and distal ends and central axes extending from the proximal end to the distal end. A first annular element may be located at the distal end of the elongate tube, the first annular element may have a flat portion at its distal end that is oriented perpendicular to the central axis, the flat portion extending from an outer circumference of the first annular element to the central axis. A second annular element may also be located at the distal end of the elongate tube and be concentric with the first annular element, the second annular element may have a flat portion at its distal end that is oriented perpendicular to the central axis. In such devices at least one of the first or second annular elements is rotatable about the central axis, the rotation causing the first annular element and the second annular element to relatively pass each other to shear tissue.

The Ser. No. 13/714,285 Application:

This referenced application includes, inter alia, teachings directed to bendable medical devices such as ones for removing tissue from a subject. The devices may include a distal housing, an outer support tube, an inner drive tube, a coupler and a commutator portion. The coupler and commutator portion may serve to axially constrain a distal end of the inner drive tube during bending, and to supply fluid for lubricating, cooling and irrigating the distal end of the device.

The Ser. No. 14/033,397 Application:

This referenced application includes, inter alia, teachings directed to methods for removing at least part of a brain tumor that may first involve contacting a forward-facing tissue cutter disposed at the distal end of a tissue removal device with the brain tumor tissue. Other teachings are directed to tissue removal devices that may include a shaft having a diameter no greater than about 10 mm, and in some embodiments the tissue cutter does not extend laterally beyond the diameter of the shaft. Such methods may next involve cutting tissue from the brain tumor, using a tissue cutter. Such methods may then involve moving cut tissue through a channel of the shaft in a direction from the distal end of a tissue removal device toward a proximal end of the device.

The Ser. No. 14/440,088 Application:

This referenced application includes, inter alia, teachings directed to methods for removing a volume of tissue from a tongue in a patient to treat sleep apnea that may involve cutting tissue from the tongue using a tissue cutting device having a shaft and at least one moveable cutting member attached to the shaft at a distal end of the tissue cutting device and moving the cut tissue through a channel of the shaft in a direction from the distal end of the tissue cutting device toward a proximal end of the device. Other teachings are directed to tissue cutting devices for removing a volume of tissue from a tongue in a patient to treat sleep apnea wherein the devices may include a shaft, at least one moveable cutting member disposed at a distal end of a distal tip of the shaft, a handle coupled with a proximal portion of the shaft, and an actuator.

The Ser. No. 15/292,029 Application:

This referenced application includes, inter alia, teachings directed to Methods and devices for use in medical applications involving tissue removal. One exemplary powered scissors device includes a distal housing having a fixed cutting arm located thereon, an elongate member coupled to the distal housing and configured to introduce the distal housing to a target tissue site of the subject, a rotatable blade rotatably mounted to the distal housing, the rotatable blade having at least one cutting element configured to cooperate with the fixed arm to shear tissue therebetween, a crown gear located at a distal end of an inner drive tube, and a first spur gear configured to inter-engage with the crown gear and coupled with the rotatable blade to allow the crown gear to drive the rotatable blade.

### Aspects of the Invention

A first aspect of the invention provides a tissue cutting device, including: (a) an elongate tube having a proximal end and a distal end and a central axis extending from the proximal end to the distal end; (b) a first annular element at the distal end of the elongate tube, the first annular element having a flat portion at its distal end perpendicular to the central axis, the flat portion extending from an outer circumference of the first annular element to the central axis; and (c) a second annular element at the distal end of the elongate tube and concentric with the first annular element, the second annular element having a flat portion at its distal end perpendicular to the central axis, at least one of the first or second annular elements rotatable about the central axis, the rotation causing the first annular element and the second annular element to pass each other to shear tissue.

Numerous variations of the first aspect of the invention are possible and include, for example: (1) the elongate tube having a diameter less than 5 mm; (2) at least one of the first and second annular elements having a tooth having a radial thickness of less than 50 microns; (3) the flat portion having an axial thickness of less than 100 microns; (4) the first annular element being rotatable about the central axis in an opposite direction from the second annular element; (5) the first annular element being rotatable about the central axis in a same direction as the second annular element, the first annular element and the second annular element being configured to be rotated at different speeds; (6) further including an intake window at the distal end of the elongate tube; (7) further including a hole extending along the central axis; (8) variation (7) further including an ancillary component extending through the hole, the ancillary component including an imaging element, a guide wire, a water jet tube, or a barbed device; and (9) further including a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

A second aspect of the invention provides a tissue cutting device, including: (a) an elongate tube having a proximal end and a distal end and a central axis extending from the proximal end to the distal end; (b) a first annular element at the distal end of the elongate tube; and (c) a second annular element at the distal end of the elongate tube and concentric with the first annular element, at least one of the first or second annular elements rotatable about the central axis, the rotation causing the first annular element and the second annular element to pass each other to shear tissue; wherein the first and second elements together form a conical shape at the distal end of the elongate tube; and wherein edges of the first and second tubular element are beveled to further shear tissue.

Numerous variations of the second aspect of the invention are possible and include, for example: (1) the elongate tube having a diameter less than 5 mm; (2) the beveled edges having a thickness less than 10 microns; (3) the first annular element being rotatable about the central axis in an opposite direction from the second annular element; (4) the first and second elements together form a second conical shape, the second conical shape facing proximally; (5) the first annular element being rotatable about the central axis in a same direction as the second annular element, the first annular element and the second annular element being configured to rotate at different speeds; (6) further including an intake window at the distal end of the elongate tube; (7) further including a hole extending along the central axis; (8) variation (7) further including an ancillary component extending through the hole, the ancillary component including an imaging element, a guide wire, a water jet tube, or a barbed device; and (9) further including a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

A third aspect of the invention provides a tissue cutting device, including: (a) an elongate tube having a proximal end and a distal end and a central axis extending from the proximal end to the distal end; (b) a first annular element at the distal end of the elongate tube; and (c) a second annular element at the distal end of the elongate tube and concentric with the first annular element, at least one of the first or second annular elements rotatable about the central axis; wherein the first and second annular elements each have an axially-extending cutting surface, the rotation causing the axially-extending surfaces of the first and second annular elements to pass each other to shear tissue, and wherein the first and second annular elements each have a radially-extending cutting surface, rotation causing the axially-extending surfaces of the first and second elements to pass each other to shear tissue, wherein the axially extending cutting surface has an axial length of less than 100 microns.

Numerous variations of the third aspect of the invention are possible and include, for example: (1) further including teeth extending along the axially-extending or radially-extending cutting surfaces; (2) the elongate tube having a diameter less than 0.5 mm; (3) the first annular element being rotatable about the central axis in an opposite direction from the second annular element; (4) the first annular element being rotatable about the central axis in a same direction as the second annular element, the first annular element and the second annular element being configured to be rotated at different speeds; (5) further including an intake window at the distal end of the elongate tube; (6) further including a hole extending along the central axis; (7) variation (6) further including an ancillary component extending through the hole, the ancillary component including an imaging element, a guide wire, a water jet tube, or a barbed device; and (8) further including a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

A fourth aspect of the invention provides a tissue cutting device, including: (a) an elongate tube having a proximal end and a distal end and a central axis extending from the proximal end to the distal end; (b) a first annular element at the distal end of the elongate tube; and (c) a second annular element at the distal end of the elongate tube and concentric with the first annular element, at least one of the first or second annular elements rotatable about the central axis; wherein the first and second annular elements each include axially-extending teeth, the teeth having a radial thickness of less than 10 microns, the rotation causing the teeth of the first annular element and the teeth of the second annular element to pass each other to shear tissue.

Numerous variations of the fourth aspect of the invention are possible and include, for example: (1) the elongate tube having a diameter less than 5 mm; (2) the first annular element being rotatable about the central axis in an opposite direction from the second annular element; (3) the first annular element being rotatable about the central axis in a same direction as the second annular element, the first annular element and the second annular element being configured to be rotated at different speeds; (4) the teeth having a pitch of less than 200 microns; (5) further including an intake window at the distal end of the elongate tube; (6) further including a hole extending along the central axis; (7) variation (6) further including an ancillary component extending through the hole, the ancillary component including an imaging element, a guide wire, a water jet tube, or a barbed device; and (8) further including a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

A fifth aspect of the invention provides a tissue cutting device, including: (a) an elongate tube having a proximal end and a distal end and a central axis extending from the proximal end to the distal end; (b) a first annular element at the distal end of the elongate tube, the first annular element including a plurality of first shearing elements, each first shearing element having a perpendicular shearing surface that is perpendicular to the central axis; and (c) a second annular element at the distal end of the elongate tube and concentric with the first annular element, the second annular element including a plurality of second shearing elements, each second shearing element having a perpendicular shearing surface that is perpendicular to the central axis, wherein at least one of the first or second annular elements is rotatable about the central axis, the rotation causing the perpendicular shearing surfaces of the first shearing elements and the perpendicular shearing surfaces of the second shearing elements to pass each other to shear tissue.

Numerous variations of the fifth aspect of the invention are possible and include, for example: (1) at least some of the perpendicular shearing surfaces of the first shearing elements lying along the same plane; (2) variation (1) wherein the at least some of the perpendicular shearing surfaces are located at the same radial distance from the central axis; (3) at least some of the perpendicular shearing surfaces not lying along the same plane; (4) variation (3) wherein the at least some perpendicular shearing surfaces are located at different radial distances from the central axis; (5) each first shearing element having a parallel shearing surface that is parallel to the central axis, each second shearing element having a parallel shearing surface that is parallel to the central axis, and rotation of one or both of the first and second annular element causing the parallel shearing surfaces of the first shearing elements and the parallel shearing surfaces of the second shearing elements to pass each other to shear tissue; (6) variation (5) wherein at least some of the parallel shearing surfaces of the first shearing elements lie along the same radial plane; (7) variation (6) wherein the at least some parallel shearing surfaces are spaced apart from each other circumferentially; (8) variation (4) wherein at least some of the parallel shearing surfaces of the first shearing elements are spaced apart from each other radially; and (9) the elongate tube having a diameter of less than 5 mm.

A sixth aspect of the invention provides a tissue cutting device, including: (a) an elongate tube having a proximal end and a distal end and a central axis extending from the proximal end to the distal end; (b) a first annular element at the distal end of the elongate tube, the first annular element including a plurality of first shearing elements, each first shearing element having a parallel shearing surface that is parallel to the central axis; and (c) a second annular element at the distal end of the elongate tube and concentric with the first annular element, the second annular element including a plurality of second shearing element, each second shearing element having a parallel shearing surface that is parallel to the central axis, wherein at least one of the first or second annular elements is rotatable about the central axis, the rotation causing the parallel shearing surfaces of the first shearing elements and the parallel shearing surfaces of the second shearing elements to pass each other to shear tissue.

Numerous variations of the sixth aspect of the invention are possible and include, for example: (1) at least some of the parallel shearing surfaces of the first shearing elements lying along the same radial plane; (2) variation (1) wherein the at least some of the parallel shearing surfaces are spaced apart from each other axially; (3) variation (2) wherein the at least some of the parallel shearing surfaces are spaced apart from each other circumferentially; (4) at least some of the parallel shearing surfaces of the first shearing elements being spaced apart from each other radially; and (5) the elongate tube having a diameter of less than 5 mm.

A seventh aspect of the invention provides a medical device for removing tissue from a subject, including: (a) a distal housing configured with a tissue cutter assembly; (b) an elongate member coupled to the distal housing and configured to introduce the distal housing to a target tissue site of the subject, the elongate member having an outer tube, an inner drive tube rotatably mounted within the outer tube, and an annular void formed between the inner drive tube and the outer tube, wherein the outer tube and the distal housing form a stator assembly; (c) a coupler located at a distal end of the inner drive tube and rotationally coupled therewith to form a rotor assembly, the coupler configured to engage with the tissue cutter assembly to rotatably drive the tissue cutter assembly, the coupler having a rear thrust surface configured to cooperate with a first surface on the stator assembly to prevent the inner drive tube from moving proximally beyond a predetermined rear location, the coupler having a forward thrust surface configured to cooperate with a second surface on the stator assembly to prevent the inner drive tube from moving distally beyond a predetermined forward location; and (d) a commutator portion located between the rotor assembly and the stator assembly, the commutator portion having at least one solid region configured to rotatably support the rotor assembly relative to the stator assembly, the commutator portion having at least one fluid channel configured to allow passage of a fluid from the annular void, distally across the commutator portion, and into a first fluid plenum adjacent to the rear thrust surface and the first surface of the stator assembly; wherein the coupler and the distal housing form at least one passage therebetween that fluidically connects the first fluid plenum with a second fluid plenum adjacent to the forward thrust surface and the second surface of the stator assembly, wherein the device is configured to allow a fluid to flow distally through the annular void, through the at least one fluid channel in the commutator portion, through the first fluid plenum, through the at least one passage between the coupler and the distal housing, through the second fluid plenum, into at least a portion of the tissue cutter assembly, and proximally through the inner drive tube, wherein the device is configured to allow the fluid to lubricate and cool the forward and rear thrust surfaces and the tissue cutter assembly, and to transport tissue pieces cut by the tissue cutter assembly proximally through the inner drive tube.

Numerous variations of the seventh aspect of the invention are possible and include, for example: (1) the commutator portion being located on the coupler; (2) the commutator portion being located on the distal housing; (3) the commutator portion being located on both the coupler and the distal housing; (4) the commutator portion including a radially outwardly protruding bearing surface configured to rotate relative to and bear against a portion of the stator assembly, and a radially inwardly protruding surface at least partially defining the at least one fluid channel across the commutator portion; (5) the coupler being integrally formed on the distal end of the inner drive tube; (6) the coupler being a separate piece attached to the distal end of the inner drive tube; (7) the rotor assembly including a third plenum axially located between the first plenum and the second plenum; (8) variation (7) wherein the third plenum is formed in the coupler and encircles the coupler; (9) variation (7) wherein the third plenum is formed in the distal housing and encircles the distal housing; (10) the inner drive tube having a proximal end that is axially unconstrained so that it may move axially relative to a proximal end of the outer tube; (11) at least a portion of both the inner drive tube and outer tube being bendable; (12) variation (11) wherein at least a portion of at least one of the inner drive tube and outer tube is malleable; (13) a first portion of the elongate member telescoping within a second portion of the elongate member; and (14) a first portion of the elongate member articulating around at least one transverse pivot axis relative to a second portion of the elongate member.

An eighth aspect of the invention provides a medical device for removing tissue from a subject, including: (a) a distal housing configured with a tissue cutter assembly; (b) an elongate member coupled to the distal housing and configured to introduce the distal housing to a target tissue site of the subject, the elongate member having an outer tube, an inner drive tube rotatably mounted within the outer tube, and an annular void formed between the inner drive tube and the outer tube, wherein the outer tube and the distal housing form a stator assembly; (c) a crown gear located on a distal end of the inner drive tube, the coupler configured to engage a right angle gear of the tissue cutter assembly to rotatably drive the tissue cutter assembly, (d) a thrust ring rigidly affixed around the inner drive tube near the distal end of the drive tube, the thrust ring having a rear thrust surface configured to cooperate with a first surface on the stator assembly to prevent the inner drive tube from moving proximally beyond a predetermined rear location; and (e) a commutator portion located between the inner drive tube and the stator assembly, the commutator portion having at least one solid region configured to rotatably support the inner drive tube relative to the stator assembly, the commutator portion having at least one fluid channel configured to allow passage of a fluid from the annular void, distally across the commutator portion, and into a first fluid plenum adjacent to the rear thrust surface and the first surface of the stator assembly; wherein the thrust ring and the distal housing form at least one passage therebetween that is in fluid communication with the first fluid plenum, wherein the device is configured to allow a fluid to flow distally through the annular void, through the at least one fluid channel in the commutator portion, through the first fluid plenum, through the at least one passage between the thrust ring and the distal housing, into at least a portion of the tissue cutter assembly, and proximally through the inner drive tube, wherein the device is configured to allow the fluid to lubricate and cool the rear thrust surface and the tissue cutter assembly, and to transport tissue pieces cut by the tissue cutter assembly proximally through the inner drive tube.

Numerous variations of the eighth aspect of the invention are possible and include, for example: (1) the commutator portion being located on the distal housing; (2) the commutator portion including a radially inwardly protruding bearing surface configured to bear against a portion of the inner drive tube, thereby radially constraining the inner drive tube while permitting it to freely rotate, and a radially outwardly protruding surface at least partially defining the at least one fluid channel across the commutator portion; (3) the thrust ring being rigidly affixed to the inner drive tube with at least one weldment inside a preformed hole through a wall of the thrust ring; (4) the first fluid plenum being formed in the distal housing and encircles the distal housing; (5) the crown gear and the right angle gear being configured to cooperate to prevent the inner drive tube from moving distally beyond a predetermined forward location; (6) the tissue cutter assembly including a first rotor and a second, oppositely rotating rotor, each of the first and second rotors configured to rotate about an axis that is perpendicular to a central longitudinal axis of the elongate member, each of the first and second rotors having a plurality of blades, wherein the blades of the first rotor are configured to interdigitate with the blades of second rotor; (7) the inner drive tube having a proximal end that is axially unconstrained so that it may move axially relative to a proximal end of the outer tube; (8) at least a portion of both the inner drive tube and outer tube being bendable; (9) variation (8) wherein at least a portion of at least one of the inner drive tube and outer tube is malleable; (10) a first portion of the elongate member telescoping within a second portion of the elongate member; and (11) a first portion of the elongate member articulating around at least one transverse pivot axis relative to a second portion of the elongate member.

A ninth aspect of the invention provides a method for removing at least part of a pituitary tumor in a patient, the method including: (a) advancing a distal end of a tissue cutter through a nostril and through the sphenoid sinus of the patient to contact a cutting member of the tissue cutter with the pituitary tumor, wherein the tissue cutter includes an outer shaft configured to enter the nostril and having an outer diameter no greater than about 10 mm, which includes a distal shaft portion and a proximal shaft portion, and wherein the distal shaft portion is sharply angled relative to the proximal shaft portion; (b) activating the cutting member to cut tissue from the pituitary tumor by rotating an inner drive shaft located within the outer shaft; and (c) moving the cut pituitary tumor tissue through a channel within at least one of the shafts toward a proximal end of the tissue cutter.

Numerous variations of the ninth aspect of the invention are possible and include, for example: (1) the cutting member not extending laterally beyond the outer diameter of the tissue cutter outer shaft; (2) further including, before contacting the pituitary tumor: forming an opening through the sphenoid sinus; and advancing the distal end of the tissue cutter through the opening; (3) variation (2) wherein the opening is formed using the tissue cutter; (4) cutting the tissue including shredding the tissue; (5) moving the tissue including urging the tissue into the channel with a cutting motion of the tissue cutter; (6) variation (5) wherein moving the cut tissue through the channel further includes applying suction to the channel; (7) variation (6) wherein moving the cut tissue through the channel further includes introducing fluid, via the tissue cutter, to an area at or near the distal end of the tissue cutter, wherein the applied suction moves at least some of the fluid proximally through the channel with the cut tissue; (8) the cutting member including at least one moveable blade and at least one stationary blade, and wherein cutting tissue including rotating the at least one rotating blade past the at least one stationary blade; (9) the cutting member including at least two interdigitated blades, and cutting tissue including rotating the two interdigitated blades toward one another to shear tissue therebetween; (10) the cutting member being selected from the group consisting of micro-shears, graspers and biopsy forceps; (11) the distal shaft portion being angled relative to the proximal shaft portion by at least 1 degree; (12) the distal shaft portion being angled relative to the proximal shaft portion by at least 45 degrees; (13) the distal shaft portion being angled relative to the proximal shaft portion by about 90 degrees; (14) variation (11) wherein the proximal shaft portion is curved; (15) further including visualizing the tissue cutting using a visualization device selected from the group consisting of a straight endoscope, an angled endoscope, a swing prism endoscope, a side viewing endoscope, a flexible endoscope, a CMOS digital camera, an ultrasound device and a scanning single fiber endoscope; (16) variation (13) wherein the visualization device is incorporated into the tissue removal device; (17) further including measuring an amount of the removed tissue by filtering the removed tissue from a stream of irrigation fluid; (18) further including measuring an amount of the removed tissue by determining motor torque in the tissue removal device during engagement of the device with the tissue and using at least one of the determined motor torque, a time period of tissue removal or a loading condition to approximate the amount of the removed tissue; (19) further including monitoring a location of the tissue removal device during use, using a navigation system and at least one tracking feature on the device; (20) further including collecting a sample of cut tissue, using a tissue capturing feature on the device, for use as a histological sample; (21) further including at least partially removing a blood clot from the patient through the channel, wherein removing the blood clot includes breaking up the clot using the cutting member; (22) the tissue cutter being coupled with an image guided or robotic surgical system during performance of at least part of the method; (23) further including protecting tissues not intended for treatment from contacting the cutting member during use of the device; and (24) further including: stimulating a portion of the pituitary tumor using a stimulation member at or near the distal end of the tissue removal device; and deciding whether to cut the stimulated tissue, based on an observed response from the stimulation.

A tenth aspect of the invention provides a device for removing at least part of a pituitary tumor, the device including: (a) an outer shaft including a distal end, a proximal end, a distal shaft portion, a proximal shaft portion, a sharp bend at a juncture of the distal shaft portion and the proximal shaft portion, a channel extending from the distal end through at least part of the proximal portion, and an outer diameter no greater than about 10 mm; (b) at least one moveable cutting member disposed at the distal end of the shaft such that, in use, the cutting member does not extend laterally beyond the outer diameter of the outer shaft; (c) a handle coupled with the proximal portion of the outer shaft; (d) an actuator coupled with the handle and the at least one cutting member to allow a user to activate the at least one cutting member via the handle, the actuator including an inner drive shaft configured to rotate about a central longitudinal axis when activating the at least one cutting member; and (e) at least one aperture on at least one of the handle or the proximal shaft portion and in fluid communication with the channel, for providing at least one of attachment to a source of suction force or withdrawal of cut tissue through the aperture.

Numerous variations of the tenth aspect of the invention are possible and include, for example: (1) the distal portion having a length of no more than about 25 mm, and the bend forming an angle between the distal shaft portion and the proximal shaft portion of at least about 5 degrees; (2) the channel extending from the distal end of the outer shaft to the at least one aperture; (3) further including a suction port on the proximal portion or the handle for applying suction to the channel; (4) variation (3) further including an irrigation port on the proximal portion or the handle for applying irrigation fluid to the channel; (5) variation (4) wherein the suction port is in fluid communication with the channel which serves as a suction channel in the inner drive shaft of the device, and wherein the irrigation port is in fluid communication with an irrigation channel including a space between an outer surface of the inner tube and an inner surface of the outer shaft of the device; (6) the at least one moveable cutting member including: at least one rotating blade, and at least one stationary blade positioned relative to the rotating blade such that tissue is cut between the rotating blade and the stationary blade; (7) the at least one moveable cutting member including multiple interdigitated blades that rotate toward one another to shred tissue; (8) the at least one moveable cutting member being selected from the group consisting of micro-shears, graspers and biopsy forceps; (9) further including at least one tubular crown gear for driving the at least one cutting member; (10) variation (9) wherein the at least one tubular crown gear includes two tubular crown gears coupled together with at least one intermediate gear disposed between them; (11) variation (10) wherein the intermediate gear is disposed at the bend in the outer shaft; (12) further including an energy transmission member coupled with the distal tip of the outer shaft for transmitting energy to the pituitary tumor, wherein the energy transmitted by the energy transmission member is selected from the group consisting of radiofrequency, ultrasound, microwave, heat and laser energy; (13) further including a visualization lumen coupled with an outer surface of the outer shaft, for holding at least a portion of an elongate visualization device; (14) the proximal portion of the outer shaft being curved; (15) further including at least one attachment member for attaching the device to an image guide or robotic surgical system; and (16) the distal shaft portion including a safety portion that extends along one side of the cutting member to prevent tissues not intended for treatment from contacting the cutting member during use of the device.

An eleventh aspect of the invention provides a method for removing a volume of tissue from a tongue in a patient to treat sleep apnea, the method including: (a) cutting tissue from the tongue using a tissue cutting device having a shaft and at least one moveable cutting member attached to the shaft at a distal end of the tissue cutting device; and (b) moving the cut tissue through a channel of the shaft in a direction from the distal end of the tissue cutting device toward a proximal end of the device.

Numerous variations of the eleventh aspect of the invention are possible and include, for example: (1) further including, before cutting the tissue: forming an incision in the tongue, and advancing the distal end of the tissue cutting device through the incision to cut tissue within an inner portion of the tongue; (2) variation (1) wherein the incision is formed using the tissue cutting device; (3) variation (1) wherein the incision is formed in a top of the tongue; (4) variation (1) wherein the incision is formed in a bottom of the tongue; (5) variation (1) wherein the incision is formed from under the patient's chin through a bottom of the tongue; (6) variation (1) further including closing the incision using an energy emitting member on the tissue cutting device, wherein the energy emitting member emits energy selected from the group consisting of radiofrequency, ultrasound, microwave, heat and laser energy; (7) the moveable cutting member including at least one moveable blade and at least one stationary blade, and cutting tissue including rotating the at least one rotating blade past the at least one stationary blade; (8) the moveable cutting member including at least two interdigitated tissue cutters, and cutting tissue including rotating the two interdigitated cutters toward one another; (9) moving the cut tissue through the channel including applying suction to the channel; (10) variation (9) wherein moving the cut tissue through the channel further includes introducing fluid, via the tissue cutting device, to an area at or near the distal end of the tissue cutting device, wherein the applied suction moves at least some of the fluid proximally through the channel with the cut tissue; (11) the shaft of the tissue cutting device having a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees; (12) further including visualizing the cutting using a visualization device selected from the group consisting of a straight endoscope, an angled endoscope, a swing prism endoscope, a side viewing endoscope, a flexible endoscope, a CMOS digital camera, an ultrasound device and a scanning single fiber endoscope; (13) variation (12) wherein the visualization device is incorporated into the tissue removal device; (14) further including measuring an amount of the removed tissue by filtering the removed tissue from a stream of irrigation fluid; and (15) further including measuring an amount of the removed tissue by determining motor torque in the tissue removal device during engagement of the device with the tissue and using at least one of the determined motor torque, a time period of tissue removal or a loading condition to approximate the amount of the removed tissue.

A twelfth aspect of the invention provides a method for removing a volume of tissue from a tongue in a patient to treat sleep apnea, the method including cutting tissue from the tongue using a mechanical, tissue debriding device including at least one moveable blade.

A thirteenth aspect of the invention provides a device for removing a volume of tissue from a tongue in a patient to treat sleep apnea, the device including: (a) a shaft having a proximal portion, a distal tip disposed at an angle relative to the proximal portion, and a channel extending from a distal end of the distal tip through at least part of the proximal portion; (b) at least one moveable cutting member disposed at the distal end of the distal tip and including at least two interdigated blades; (c) a handle coupled with the proximal portion of the shaft; and (d) an actuator coupled with the handle for actuating the at least one moveable cutting member.

Numerous variations of the thirteenth aspect of the invention are possible and include, for example: (1) the shaft having a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees; (2) the channel including a tissue removal channel extending from the distal end of the distal tip to a proximal aperture on the proximal portion through which tissue can be removed from the device; (3) further including a suction port on the proximal portion or the handle for applying suction to the channel; (4) variation (3) further including an irrigation port on the proximal portion or the handle for applying irrigation fluid to the channel; (5) variation (4) wherein the suction port is in fluid communication with a suction channel in an inner tube of the device, and wherein the irrigation port is in fluid communication with an irrigation channel including a space between an outer surface of the inner tube and an inner surface of the shaft of the device; (6) the at least one moveable cutting member including: at least one rotating blade, and at least one stationary blade positioned relative to the rotating blade such that tissue is cut between the rotating blade and the stationary blade; (7) the at least one moveable cutting member including multiple interdigitated cutters that rotate toward one another to shred tissue; (8) the at least one moveable cutting member including multiple interdigitated cutters that rotate toward one another to shred tissue; (9) variation (8) wherein the at least one tubular crown gear includes two tubular crown gears coupled together with at least one intermediate gear disposed between them; (10) variation (9) wherein the intermediate gear is disposed at a bend in the shaft located at an intersection of the proximal portion and the distal tip; and (11) further including an energy transmission member coupled with the distal tip of the shaft for transmitting energy to the tissue, wherein the energy transmitted by the energy transmission member is selected from the group consisting of radiofrequency, ultrasound, microwave, heat and laser energy.

A fourteenth aspect of the invention provides a system for removing a volume of tissue from a tongue in a patient to treat sleep apnea, the system including: (a) a mechanical tissue debrider, including: (i) a shaft having a proximal portion, a distal tip disposed at an angle relative to the proximal portion, and a channel extending from a distal end of the distal tip through at least part of the proximal portion; (ii) at least one moveable cutting member disposed at the distal end of the distal tip; (iii) a handle coupled with the proximal portion of the shaft; and (iv) an actuator coupled with the handle for actuating the at least one moveable cutting member; and (b) an energy transmission member coupled with the distal tip of the shaft for transmitting an energy to the tissue, wherein the energy is selected from the group consisting of radiofrequency, ultrasound, microwave, heat and laser energy.

Numerous variations of the fourteenth aspect of the invention are possible and include, for example: (1) further including a suction port on the proximal portion of the shaft or the handle for applying suction to the channel; and (2) variation (1) further including an irrigation port on the proximal portion of the shaft or the handle for applying irrigation fluid to the channel.

A fifteenth aspect of the invention provides a powered scissors device including: (a) a distal housing having a fixed cutting arm located thereon; (b) an elongate member coupled to the distal housing and configured to introduce the distal housing to a target tissue site of the subject, the elongate member including an outer tube and an inner drive tube rotatably mounted within the outer tube; (c) a rotatable blade rotatably mounted to the distal housing, the rotatable blade having at least one cutting element configured to cooperate with the fixed arm to shear tissue therebetween; (d) a crown gear located at a distal end of the inner drive tube; and (e) a first spur gear configured to inter-engage with the crown gear and coupled with the rotatable blade to allow the crown gear to drive the rotatable blade.

Numerous variations of the fifteenth aspect of the invention are possible and include, for example: (1) the rotatable blade having an axis of rotation that is perpendicular to an axis of rotation of the inner drive tube; (2) the rotatable blade being partially located within a slot formed within the distal housing such that the at least one cutting element is covered by the distal housing during at least half of its rotation about an axis of rotation of the rotatable blade; (3) the rotatable blade having multiple cutting elements, each of the cutting elements having a cutting edge configured to cooperate with a cutting edge of the fixed arm to shear tissue therebetween; (4) variation (3) wherein every cutting edge of the multiple cutting elements of the rotatable blade lies in a common plane; (5) the cutting element being shorter than the fixed arm; (6) the cutting element having a top side and a bottom side, being flat on the top side, and having a cutting bevel provided along the bottom side; (7) the cutting element having a cutting edge that is curved, and the fixed arm having a cutting edge that is curved in the same direction; (8) variation (7) wherein the cutting edges of the cutting element and the fixed arm are curved in an outward direction trailing away from a direction of rotation of the cutting element; (9) variation (7) wherein the cutting edge of the cutting element has a smaller radius of curvature than a radius of curvature of the cutting edge of the fixed arm; and (10) the fixed arm being provided with a radio frequency electrode.

A sixteenth aspect of the invention provides a medical device for manipulating tissue of a subject, including (a) a distal housing configured with an end effector; (b) an elongate member coupled to the distal housing and configured to introduce the distal housing to a target tissue site of the subject, the elongate member including a proximal portion having a first central axis and a distal portion having a second central axis, the proximal portion of the elongate member including a proximal outer tube and a proximal inner drive tube rotatably mounted within the proximal outer tube, the distal portion of the elongate member including a distal outer tube and a distal inner drive tube rotatably mounted within the distal outer tube, the distal inner drive tube engaging with a portion of the end effector to drive the end effector; (c) a joint mechanism configured to pivotably connect a distal end of the proximal outer tube with a proximal end of the distal outer tube, wherein the joint mechanism allows the distal portion of the elongate member to be pivoted relative to the proximal portion such that an angle formed between the first and the second central axes can be changed; (d) a proximal crown gear located at a distal end of the proximal inner drive tube; (e) a distal crown gear located at a proximal end of the distal inner drive tube; and (f) a first spur gear spanning between and inter-engaging with the proximal crown gear and the distal crown gear, thereby allowing the end effector to be positioned by the proximal and the distal outer tubes, and to be driven by the proximal inner drive tube, the spur gear and the distal inner drive tube.

Numerous variations of the sixteenth aspect of the invention are possible and include, for example: (1) the end effector including a rotary tissue cutter assembly; (2) variation (1) wherein the rotary tissue cutter assembly includes at least one rotatable member that rotates about the second central axis; (3) variation (1) wherein the rotary tissue cutter assembly includes at least one rotatable member that has an axis of rotation that is perpendicular to the second central axis; (4) variation (1) wherein the distal inner drive tube includes a first lumen and the proximal inner drive tube includes a second lumen, wherein the first lumen is in fluid communication with the tissue cutter assembly and the second lumen is in fluid communication with the first lumen through the joint mechanism; (5) variation (4) wherein the tissue cutter assembly, the first lumen, the joint mechanism and the second lumen are configured to cooperate to transport tissue debris cut by the tissue cutter assembly in a proximal direction through the first lumen, the joint mechanism and the second lumen; (6) the end effector including a pair of scissor blades configured to cut tissue; (7) the end effector including a pair of tissue grasper jaws; (8) the end effector including a needle driver; (9) the proximal portion of the elongate member further including a proximal inner articulation tube rotatably mounted within the proximal outer tube, and the proximal inner articulation tube including a crown gear on a distal end thereof configured to mesh with a gear segment of the joint mechanism to pivotably drive the distal portion of the elongate member relative to the proximal portion of the elongate member; (10) the proximal portion of the elongate member including a second proximal inner drive tube rotatably mounted within the proximal outer tube, the distal portion of the elongate member including a second distal inner drive tube rotatably mounted within the distal outer tube, the second distal inner drive tube engaging with a portion of the end effector to drive the end effector, the device further including a second proximal crown gear located at a distal end of the second proximal inner drive tube, a second distal crown gear located at a proximal end of the second distal inner drive tube, and a second spur gear spanning between and inter-engaging with the second proximal crown gear and the second distal crown gear; (11) variation (10) wherein the end effector includes a pair of tissue grasper jaws, wherein one of the pair of tissue grasper jaws is configured to be rotatably driven by a crown gear located on a distal end of the first distal inner drive tube, and wherein the other of the pair of tissue grasper jaws is configured to be rotatably driven by a crown gear located on a distal end of the second distal inner drive tube, such that each of the pair of tissue grasper jaws may be independently rotated relative to the second central axis and may be rotated between an open jaw position and a closed jaw position; (12) the proximal portion of the elongate member including a second proximal drive tube rotatably mounted coaxially with the proximal outer tube, the distal portion of the elongate member including a second distal drive tube rotatably mounted coaxially with the distal outer tube, the second distal drive tube engaging with a portion of the end effector to support the end effector, the device further including a second proximal crown gear located at a distal end of the second proximal drive tube, a second distal crown gear located at a proximal end of the second distal drive tube, and a second spur gear spanning between and inter-engaging with the second proximal crown gear and the second distal crown gear, and the rotational orientation of the end effector relative to the second central axis is changeable by rotating the second distal drive tube with the second proximal drive tube and second spur gear; (13) variation (12) wherein the proximal and the distal portions of the elongate member are configured to rotate together about the first central axis relative to a more proximal portion of the device; (14) variation (12) wherein the proximal and the distal portions of the elongate member are configured to translate together about the first central axis relative to a more proximal portion of the device; (15) variation (12) wherein the proximal and the distal portions of the elongate member are configured to pivot together about a shoulder joint relative to a more proximal portion of the device; (16) variation (12) wherein the proximal and the distal portions of the elongate member are configured to translate together in a direction perpendicular to the first central axis relative to a more proximal portion of the device; (17) variation (12) wherein the proximal and the distal portions of the elongate member are configured to pivot together about an axis perpendicular to the first central axis relative to a more proximal portion of the device; and (18) further including a second spur gear spanning between and inter-engaging with the proximal crown gear and the distal crown gear, thereby allowing the end effector to be driven by the proximal inner drive tube, the first and second spur gears and the distal inner drive tube, wherein the first and the second spur gears provide a dual load path between the proximal and the distal inner drive tubes

A seventeenth aspect of the invention provides a method of manipulating tissue of a subject including: (a) providing a device having a distal housing configured with an end effector and an elongate member coupled to the distal housing; (b) introducing the distal housing to a target tissue site of the subject with the elongate member; (c) driving the end effector with a drive train including a proximal crown gear located at a distal end of a proximal drive tube, a distal crown gear located at a proximal end of a distal drive tube, and a first spur gear spanning between and inter-engaging with the proximal crown gear and the distal crown gear; (d) pivoting the location of the end effector, the distal housing and the distal drive tube relative to the proximal drive tube by rotating a second proximal tube, the second proximal tube being rotatably mounted coaxially with the proximal drive tube and having a crown gear located on a distal end, the crown gear engaging with a gear segment coaxially mounted with the spur gear; and (e) manipulating the tissue of the subject with the end effector.

Numerous variations of the seventeenth aspect of the invention are possible and include, for example: (1) the end effector including a rotary tissue cutter assembly; (2) variation (1) wherein the rotary tissue cutter assembly includes at least one rotatable member that rotates about a central axis of the distal drive tube; (3) variation (1) wherein the rotary tissue cutter assembly includes at least one rotatable member that has an axis of rotation that is perpendicular to a central axis of the distal drive tube; (4) the end effector including a pair of scissor blades configured to cut tissue; (5) the end effector including a pair of tissue grasper jaws; (6) the end effector including a needle driver; and (7) the pivoting step including a computer receiving movement inputs from a surgeon and providing electrical outputs to drive an electric motor coupled to the second proximal tube.

An eighteenth aspect of the invention provides a powered scissors device including: (a) a distal housing having a fixed cutting arm located thereon; (b) an elongate member coupled to the distal housing and configured to introduce the distal housing to a target tissue site of the subject, the elongate member including an outer tube and an inner drive tube rotatably mounted within the outer tube; (c) a rotatable blade rotatably mounted to the distal housing, the rotatable blade having at least one cutting element configured to cooperate with the fixed arm to shear tissue therebetween; (d) a crown gear located at a distal end of the inner drive tube; and (e) a first spur gear configured to inter-engage with the crown gear and coupled with the rotatable blade to allow the crown gear to drive the rotatable blade.

Numerous variations of the eighteenth aspect of the invention are possible and include, for example: (1) the rotatable blade having an axis of rotation that is perpendicular to an axis of rotation of the inner drive tube; and (2) the rotatable blade being partially located within a slot formed within the distal housing such that the at least one cutting element is covered by the distal housing during at least half of its rotation about an axis of rotation of the rotatable blade.

A nineteenth aspect of the invention provides a medical device for manipulating tissue of a subject, including: (a) a distal housing configured with an end effector, the end effector including a first member pivotably mounted to the distal housing and a second member pivotably mounted to the distal housing independent from the first member; the first and the second members each having surfaces configured to manipulate tissue of the subject; and (b) an elongate member coupled to the distal housing and configured to introduce the distal housing to a target tissue site of the subject, the elongate member including a first drive tube and a second drive tube coaxially mounted within the first drive tube, the first and the second drive tubes being configured to independently rotate relative to the distal housing, the first drive tube having a first crown gear located on a distal end thereof coupled with the first member such that rotating the first drive tube and first crown gear causes the first member to pivot, the second drive tube having a second crown gear located on a distal end thereof coupled with the second member such that rotating the second drive tube and second crown gear causes the second member to pivot, wherein the tissue engaging surfaces of the first and the second members may be alternately pivoted towards each other by their respective drive tubes into a closed position and away from each other into an open position.

Numerous variations of the nineteenth aspect of the invention are possible and include, for example: (1) the first and the second members being rotated in the same direction by their respective drive tubes such that an articulation angle of the members relative to the distal housing when in the closed position may be varied; (2) the first member and the second member both pivoting about a common axis; (3) at least one of the first and the second members pivoting about an axis that is transverse to an axis of rotation of the first and the second drive tubes; (4) the first and the second members forming tissue graspers; (5) the first and the second members forming tissue scissors; (6) further including a first gear segment coupled to the first member and configured to mesh with the first crown gear for pivotably driving the first member, and a second gear segment coupled to the second member and configured to mesh with the second crown gear for pivotably driving the second member; (7) variation (6) wherein the first and the second gear segments are located on opposite sides of a central rotation axis of the first and the second drive tubes such that the drive tubes are rotated in a common direction to drive the first and the second members from the open position to the closed position; (8) further including at least one radio frequency electrode located on one of the tissue manipulating surfaces of the first and the second members; and (9) further including a third drive tube configured to rotate the end effector relative to the elongate member.

A twentieth aspect of the invention provides a cutting device including (a) an elongate tube having a proximal end, a distal end, and a central axis extending from the proximal end to the distal end; (b) a first annular element at the distal end of the elongate tube, and (c) a second annular element at the distal end of the elongate tube, wherein the first annular element includes at least one surface, and the at least one surface has a first shearing element, wherein the second annular element includes at least one second surface, and the at least one second surface includes a second shearing element, wherein the second annular element is concentric with the first annular element and rotatable about a central axis, and wherein rotation causes the first shearing elements and the second shearing elements to pass each other.

Numerous variations of the twentieth aspect of the invention exist and may include, for example, one or more of: (1) at least one surface being be perpendicular to the central axis; (2) at least one surface being parallel to the central axis; (3) at least a portion of the at least one surface being perpendicular and being located at the radial-most location of the first or second annular elements; (4) a total radial length occupied by the at least one perpendicular surface being selected from the group consisting of (a) at least 1/10 of the radius of the cutting device, (b) being at least ⅕ of the radius of the cutting device, (c) being at least ¼ of the radius of the cutting device, (d) being at least ⅓ of the radius of the cutting device, and (e) being such as at least ½ the radius of the cutting device; (5) at least one surface being spaced apart from the central axis; (6) at least two surfaces occupying different planes which are perpendicular to the central axis; (7) at least two surfaces being on a common plane and separated by a gap; (8) the first shearing element and the second shearing element being separated upon passing by an amount selected from the group consisting of (a) less than 20 microns, (b) less than 10 microns, (c) less than 5 microns, and (d) approximately 1 micron; (9) the first and second shearing elements contacting when passing each other; (10) the shearing elements being substantially parallel to the central axis; (11) a distance from the shearing element to the central axis being selected from the group consisting of (a) less than ⅞ of the radius, (b) less than ¾ of the radius, (c) less than ⅝ of the radius, and (d) less than ½ of the radius; (12) alternating shearing elements that are perpendicular and parallel to the central axis; and (13) each surface including a plurality of shearing elements.

A twenty-first aspect of the invention provides a cutting device including: (a) an elongate tube having a proximal end and a distal end, and a central axis extending from the proximal end to the distal end; (b) a first annular element at the distal end of the elongate tube, and (c) a second annular element at the distal end of the elongate tube; the first annular element including at least one first blade element, the at least one first blade element including a first front surface and a first back surface, the first front surface including a first front shearing element, and the first back surface including a first back shearing element; and wherein the second annular element includes at least one second blade element, the at least one second blade element including a second back surface and a second front surface, the second front surface including a second front shearing element, and the second back surface including a second back shearing element.

Numerous variations of the twenty-first aspect of the invention exist and may include, for example, one or more of: (1) the surfaces of the blades being perpendicular to the central axis; (2) the surfaces of the blades being substantially parallel to the central axis; (3) the first blade element including at least one second blade element perpendicular to the first blade element; the distance between shearing elements of the first annular element and shearing elements of the second shearing elements being selected from the group consisting of: (a) less than 20 microns, (b) less than 10 microns, (c) less than 5 microns, and (d) approximately 1 micron; (4) the shearing elements of the first annular element and the shearing elements of the second annular elements being in contact when passing each other; and (5) the surfaces of the blades having at least one tooth.

A twenty-second aspect of the invention provides a device for removing at least part of a brain tumor including: (a) a shaft having a proximal portion, a distal tip disposed at an angle relative to the proximal portion, and a channel extending from a distal end of the distal tip through at least part of the proximal portion; (b) at least one moveable cutting member disposed at the distal end of the distal tip and including at least two interdigated blades; (c) a handle coupled with the proximal portion of the shaft; and (d) an actuator coupled with the handle for actuating the at least one moveable cutting member.

Numerous variations of the twenty-second aspect of the invention exist and may include, for example, one or more of: (1) the shaft having a diameter no greater than about 10 mm; (2) the distal tip having a length of between about 1 mm and about 25 mm; (3) a bend between a proximal portion of the shaft and the distal tip forming an angle between the proximal portion and the distal tip of between about 1 degree and about 90 degrees; (4) the channel also being a tissue removal channel extending from the distal end of the distal tip to a proximal aperture on the proximal portion through which tissue can be removed from the device; (5) a suction port on the proximal portion or the handle for applying suction to the channel; (6) an irrigation port on the proximal portion or the handle for applying irrigation fluid to the channel; (7) a suction port in fluid communication with a suction channel in an inner tube of the device, (8) an irrigation port in fluid communication with an irrigation channel including a space between an outer surface of the inner tube and an inner surface of the shaft of the device; (9) the cutting member having at least one rotating blade and at least one stationary blade positioned relative to the rotating blade such that tissue is cut between the rotating blade and the stationary blade; (10) the cutting member having multiple interdigitated cutters that rotate toward one another to shred tissue; (11) the cutting member having one or more of micro-shears, graspers and/or biopsy forceps; (12) at least one tubular crown gear for driving the at least one cutting member; (13) two tubular crown gears coupled together with at least one intermediate gear disposed between them; and (14) an energy transmission member coupled with the distal tip of the shaft for transmitting energy to the brain tumor (e.g. radiofrequency, ultrasound, microwave, heat and laser energy).

A twenty-third aspect of the invention provides a system for removing at least part of a brain tumor, including: (a) a mechanical tissue debrider, including (i) a shaft having a proximal portion, (ii) a distal tip disposed at an angle relative to the proximal portion, and (iii) a channel extending from a distal end of the distal tip through at least part of the proximal portion; (b) at least one moveable cutting member disposed at the distal end of the distal tip; (c) a handle coupled with the proximal portion of the shaft; and (d) an actuator coupled with the handle for actuating the at least one moveable cutting member.

Numerous variations of the twentieth-third aspect of the invention exist and may include, for example, one or more of: (1) suction tubing for connecting the handle to a source of suction; (2) an energy transmission member coupled with the distal tip of the shaft for transmitting an energy to the tissue (e.g. radiofrequency, ultrasound, microwave, heat or laser energy); and (3) an irrigation port on the proximal portion of the shaft or the handle for applying irrigation fluid to the channel.

A twenty-fourth aspect of the invention provides a method for removing at least part of a pituitary tumor in a patient, including: (a) advancing a distal end of a tissue cutter through a nostril and through the sphenoid sinus of the patient to contact a cutting member of the tissue cutter with the pituitary tumor; and (b) activating the cutting member to cut tissue from the pituitary tumor, wherein the cutting member does not extend laterally beyond the diameter of the tissue cutter shaft; and moving the cut pituitary tumor tissue through a channel of the shaft toward a proximal end of the tissue cutter.

Numerous variations of the twenty-fourth aspect of the invention exist and include, for example, one or more of: (1) a shaft having an outer diameter no greater than about 10 mm, which includes a distal shaft portion and a proximal shaft portion; (2) variation (1) with the distal shaft portion sharply angled relative to the proximal shaft portion; (3) before contacting the pituitary tumor, forming an opening through the sphenoid sinus, and advancing the distal end of the tissue cutter through the opening; (4) variation (3) with opening formed using the tissue cutter; (5) cutting the tissue by shredding the tissue; (6) moving the tissue by urging the tissue into the channel with cutting motion of the tissue cutter; (7) moving the cut tissue through the channel by applying suction to the channel; (8) moving the cut tissue through the channel by introducing fluid, via the tissue cutter, to an area at or near the distal end of the tissue cutter, where applied suction moves at least some of the fluid proximally through the channel with the cut tissue; (9) the cutting member includes at least one moveable blade and at least one stationary blade and where cutting tissue includes rotating the at least one rotating blade past the at least one stationary blade; (10) the cutting member includes at least two interdigitated blades, and cutting tissue includes rotating the two interdigitated blades toward one another; (11) the cutting member includes one or more of micro-shears, graspers and/or biopsy forceps; (12) the distal shaft portion being angled relative to the proximal shaft portion; (13) the proximal shaft portion being curved (e.g. a gradual curve, a bayonet-shaped curve, or both); (14) visualizing the cutting using a visualization device such as, for example, a straight endoscope, an angled endoscope, a swing prism endoscope, a side viewing endoscope, a flexible endoscope, a CMOS digital camera, an ultrasound device or a scanning single fiber endoscope; (15) a visualization device incorporated into the tissue removal device; (16) measuring an amount of the removed tissue by filtering the removed tissue from a stream of irrigation fluid; (17) measuring an amount of the removed tissue by determining motor torque in the tissue removal device during engagement of the device with the tissue and using the determined motor torque, a time period of tissue removal and/or a loading condition to approximate the amount of the removed tissue; (18) monitoring a location of the tissue removal device during use, using a navigation system and at least one tracking feature on the device; (19) collecting a sample of cut tissue, using a tissue capturing feature on the device, for use as a histological sample; (20) at least partially removing a blood clot from the patient through the shaft, where removing the blood clot includes breaking up the clot using the cutting member; (21) coupling the tissue cutter to an image guided or robotic surgical system for performance of at least part of the method; (22) protecting tissues not intended for treatment from contacting the cutting member during use of the device; and (23) stimulating a portion of the pituitary tumor using a stimulation member at or near the distal end of the tissue removal device and deciding whether to cut the stimulated tissue, based on an observed response from the stimulation.

A twenty-fifth aspect of the invention provides a device for removing at least part of a pituitary tumor, including: (a) a shaft including (i) a distal end, (ii) a proximal end, (iii) a distal shaft portion, (iv) a proximal shaft portion, (v) a sharp bend at a juncture of the distal shaft portion and the proximal shaft portion, (vi) a channel extending from the distal end through at least part of the proximal portion, and (vii) an outer diameter no greater than about 10 mm; (b) at least one moveable cutting member disposed at the distal end of the shaft such that, in use, the cutting member does not extend laterally beyond the outer diameter of the shaft; (c) a handle coupled with the proximal portion of the shaft; (d) an actuator coupled with the handle and the at least one cutting member to allow a user to activate the at least one cutting member via the handle; and (e) at least one aperture on at least one of the handle or the proximal shaft portion and in fluid communication with the channel, for providing attachment to a source of suction force and/or withdrawal of cut tissue through the aperture.

Numerous variations of the twenty-fifth aspect of the invention exist and include, for example, one or more of: (1) the distal portion having a length of no more than about 25 mm and the bend having an angle between the distal shaft portion and the proximal shaft portion of at least about 5 degrees; (2) the channel extending from the distal end of the shaft to the at least one aperture; (3) a suction port on the proximal portion or the handle for applying suction to the channel; (4) an irrigation port on the proximal portion or the handle for applying irrigation fluid to the channel; (5) a suction port in fluid communication with a suction channel in an inner tube of the device, and wherein the irrigation port is in fluid communication with an irrigation channel including a space between an outer surface of the inner tube and an inner surface of the shaft of the device; (6) the moveable cutting member including at least one rotating blade and at least one stationary blade positioned relative to the rotating blade such that tissue is cut between the rotating blade and the stationary blade; (7) the moveable cutting member including multiple interdigitated blades that rotate toward one another to shred tissue; (8) the moveable cutting member including one or more of micro-shears, graspers or biopsy forceps; (9) at least one tubular crown gear for driving the at least one cutting member; (10) at least two tubular crown gears coupled together with at least one intermediate gear disposed between them for driving the at least one cutting member; (11) variation (10) with the intermediate gear disposed at the bend in the shaft; (12) an energy transmission member coupled with the distal tip of the shaft for transmitting energy to the pituitary tumor where the energy may include for example for example one or more of radiofrequency, ultrasound, microwave, heat, or laser energy; (13) a visualization lumen coupled with an outer surface of the shaft, for holding at least a portion of an elongate visualization device; (14) a proximal portion of the shaft of the device being curved; (15) at least one attachment member for attaching the device to an image guide or robotic surgical system; and (16) the distal shaft portion including a safety portion that extends along one side of the cutting member to prevent tissues not intended for treatment from contacting the cutting member during use of the device.

The disclosure of the present invention provides numerous device embodiments wherein the devices may be formed, in whole or in part, using a multi-layer, multi-material fabrication process wherein each successively formed layer includes at least two materials, one of which is a structural material and the other of which is a sacrificial material, and wherein each successive layer defines a successive cross-section of the three-dimensional structure, and wherein the forming of each of the plurality of successive layers includes: (i) depositing a first of the at least two materials; (ii) depositing a second of the at least two materials; and (B) after the forming of the plurality of successive layers, separating at least a portion of the sacrificial material from the structural material to reveal the three-dimensional structure.

Other aspects of the invention will be understood by those of skill in the art upon review of the teachings herein. Other aspects of the invention may involve combinations of the above noted aspects or variations of aspects of the invention. It is intended that variations of one aspect of the invention may be applied to other aspects of the invention and that various features of one or more aspects of the invention be useable in other aspects of the invention and even that sub-combinations of various features of one or more aspects of the invention may provide new aspects of the invention. Combinations are considered appropriate so long as the combinations do not remove all functionality provided by individual components. These other aspects of the invention may provide various combinations and sub-combination of the aspects presented above as well as provide other configurations, structures, functional relationships, processes for making, and/or procedures for using that have not been specifically set forth above.

### BRIEF DESCRIPTION OF THE DRAWINGS

FIGS. 1A-1C schematically depict side views of various stages of a CC mask plating process, while FIGS. 1D-G schematically depict a side views of various stages of a CC mask plating process using a different type of CC mask.

FIGS. 2A-2F schematically depict side views of various stages of an electrochemical fabrication process as applied to the formation of a particular structure where a sacrificial material is selectively deposited while a structural material is blanket deposited.

FIGS. 3A-3C schematically depict side views of various example subassemblies that may be used in manually implementing the electrochemical fabrication method depicted in FIGS. 2A-2F.

FIGS. 4A-4F schematically depict the formation of a first layer of a structure using adhered mask plating where the blanket deposition of a second material overlays both the openings between deposition locations of a first material and the first material itself

FIG. 4G depicts the completion of formation of the first layer resulting from planarizing the deposited materials to a desired level.

FIGS. 4H and 4I respectively depict the state of the process after formation of the multiple layers of the structure and after release of the structure from the sacrificial material.

FIGS. 5A-5E illustrate an exemplary embodiment of a cutting device as described herein.

FIGS. 6A-6C illustrate an exemplary embodiment of a cutting device as described herein.

FIGS. 7A-7B illustrate an exemplary embodiment of a cutting device described herein.

FIG. 8 illustrates an exemplary embodiment of a cutting described herein.

FIG. 9 illustrates an exemplary embodiment of a cutting device described herein.

FIGS. 10A-10B illustrate an exemplary embodiment of a cutting device described herein.

FIGS. 11A-11B illustrate an exemplary embodiment of a cutting device described herein.

FIG. 12 illustrates an exemplary embodiment of a cutting device described herein.

FIG. 13 illustrates an exemplary embodiment of a cutting device described herein.

FIG. 14 illustrates an exemplary embodiment of a cutting device described herein.

FIGS. 15A-15B illustrate an exemplary embodiment of a cutting device described herein.

FIG. 16 illustrates an exemplary embodiment of a cutting device described herein.

FIGS. 17A-17C illustrate an exemplary embodiment of a cutting device described herein.

FIG. 18 illustrates an exemplary embodiment of a cutting device described herein.

FIG. 19 illustrates an exemplary embodiment of a cutting device described herein.

FIGS. 20A-20I illustrate an exemplary embodiment of a cutting device described herein.

FIG. 21 illustrates an exemplary embodiment of a cutting device described herein.

FIGS. 22A-22B illustrate an exemplary embodiment of a cutting device described herein.

FIGS. 23A-23B illustrate an exemplary embodiment of a cutting device described herein.

FIGS. 24A-24B illustrate an exemplary embodiment of a cutting device described herein.

FIGS. 25A-25C illustrate an exemplary embodiment of a cutting device described herein.

FIGS. 26A-26C illustrate an exemplary embodiment of a cutting device described herein.

FIGS. 27A-27C illustrate an exemplary embodiment of a cutting device described herein.

FIGS. 28A-28B illustrate an exemplary embodiment of a cutting device described herein.

FIG. 29 illustrates an exemplary embodiment of a tissue cutting device having a working component extending therethrough.

FIGS. 30A-30G illustrate exemplary embodiments of working components that can extend through the medical devices described herein.

### DETAILED DESCRIPTION OF PREFERRED EMBODIMENTS

Electrochemical Fabrication in General

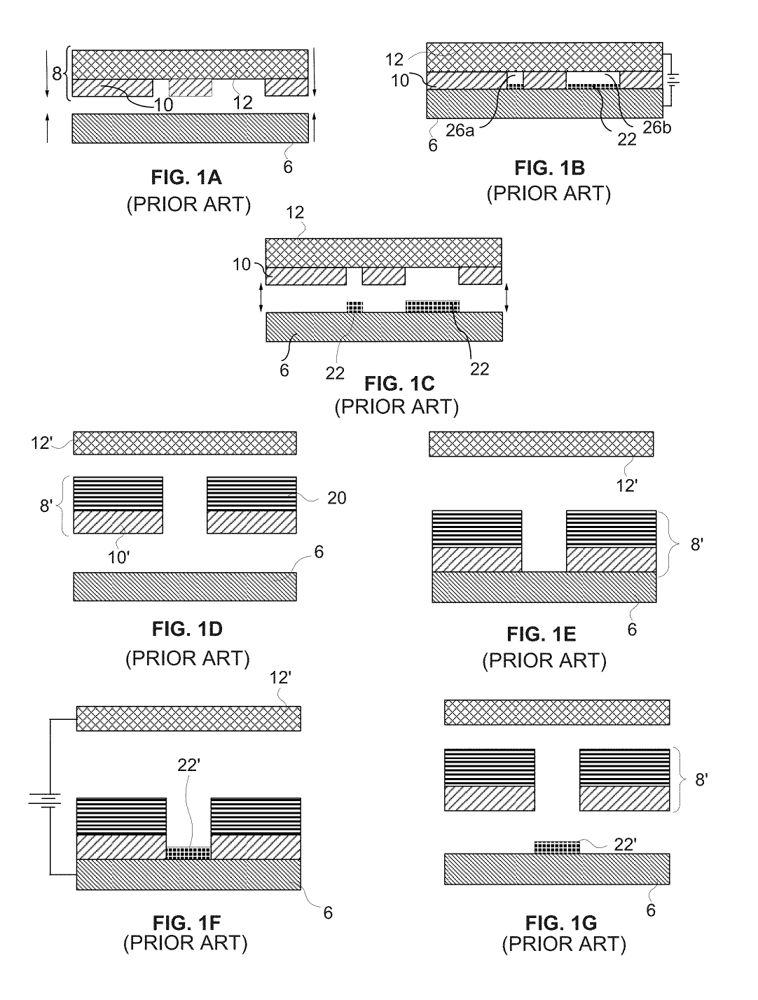

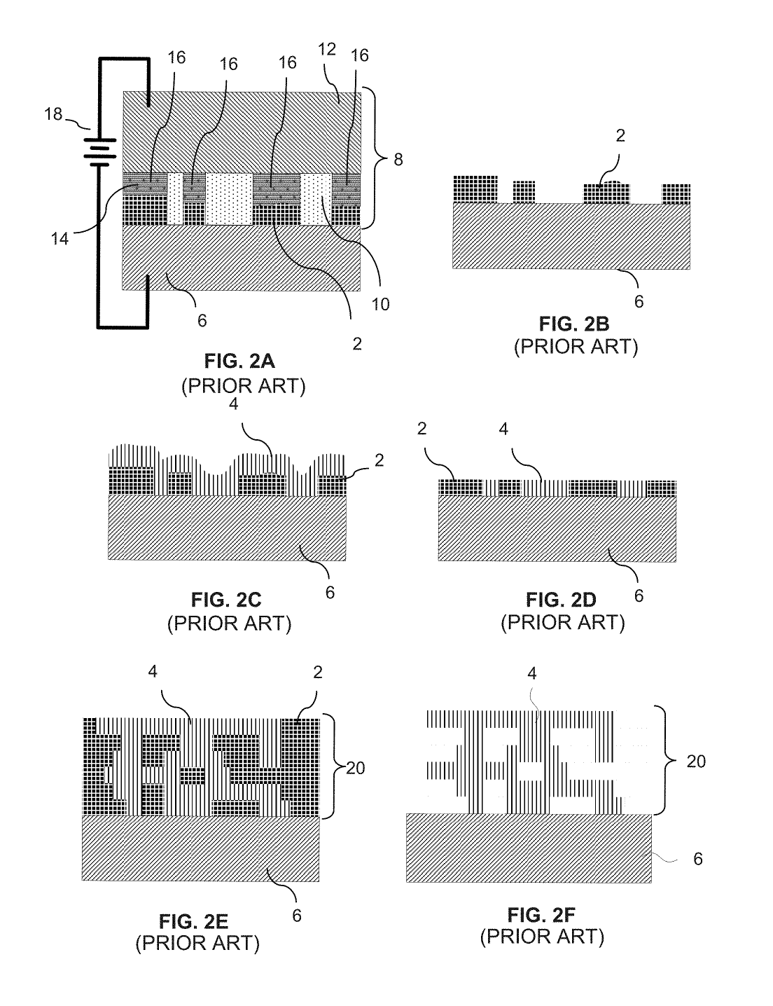

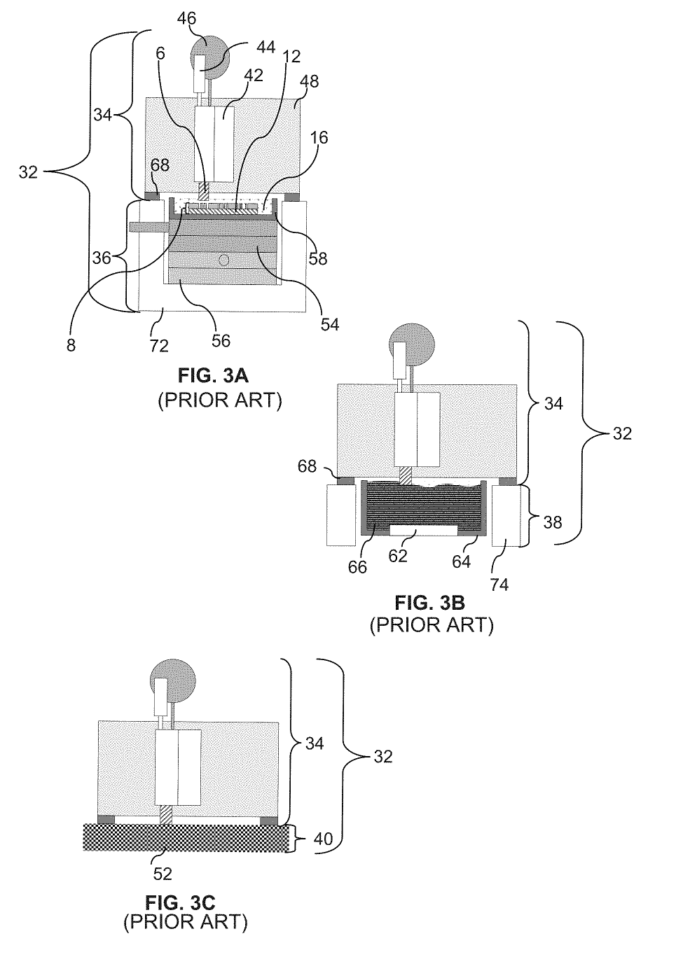

FIGS. 1A-1G, 2A-2F, and 3A-3C illustrate various features of one form of electrochemical fabrication. Other electrochemical fabrication techniques are set forth in the '630 patent referenced above, in the various previously incorporated publications, in various other patents and patent applications incorporated herein by reference. Still others may be derived from combinations of various approaches described in these publications, patents, and applications, or are otherwise known or ascertainable by those of skill in the art from the teachings set forth herein. All of these techniques may be combined with those of the various embodiments of various aspects of the invention to yield enhanced embodiments. Still other embodiments may be derived from combinations of the various embodiments explicitly set forth herein.

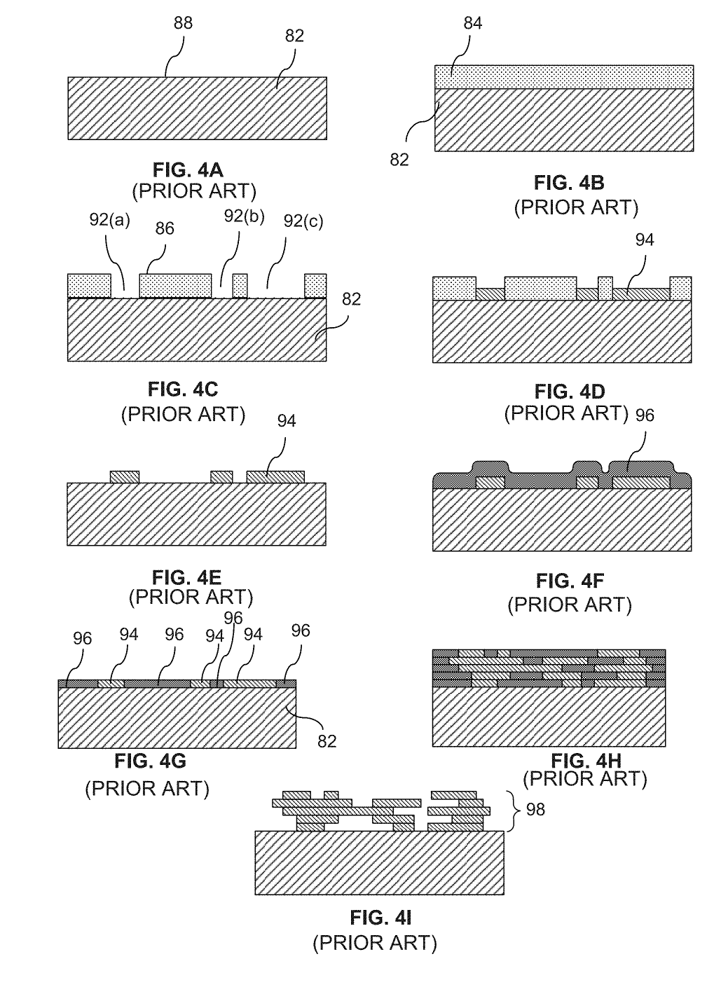

FIGS. 4A-4I illustrate various stages in the formation of a single layer of a multi-layer fabrication process where a second metal is deposited on a first metal as well as in openings in the first metal so that the first and second metal form part of the layer. In FIG. 4A a side view of a substrate 82 is shown, onto which patternable photoresist 84 is cast as shown in FIG. 4B. In FIG. 4C, a pattern of resist is shown that results from the curing, exposing, and developing of the resist. The patterning of the photoresist 84 results in openings or apertures 92(a)-92(c) extending from a surface 86 of the photoresist through the thickness of the photoresist to surface 88 of the substrate 82. In FIG. 4D a metal 94 (e.g. nickel) is shown as having been electroplated into the openings 92(a)-92(c). In FIG. 4E the photoresist has been removed (i.e. chemically stripped) from the substrate to expose regions of the substrate 82 which are not covered with the first metal 94. In FIG. 4F a second metal 96 (e.g. silver) is shown as having been blanket electroplated over the entire exposed portions of the substrate 82 (which is conductive) and over the first metal 94 (which is also conductive). FIG. 4G depicts the completed first layer of the structure which has resulted from the planarization of the first and second metals down to a height that exposes the first metal and sets a thickness for the first layer. In FIG. 4H the result of repeating the process steps shown in FIGS. 4B-4 G several times to form a multi-layer structure are shown where each layer consists of two materials. For most applications, one of these materials is removed as shown in FIG. 4I to yield a desired 3-D structure 98 (e.g. component or device).

Various embodiments of various aspects of the invention are directed to formation of three-dimensional structures from materials some of which may be electrodeposited or electroless deposited. Some of these structures may be formed form a single build level formed from one or more deposited materials while others are formed from a plurality of build layers each including at least two materials (e.g. two or more layers, more preferably five or more layers, and most preferably ten or more layers). In some embodiments, layer thicknesses may be as small as one micron or as large as fifty microns. In other embodiments, thinner layers may be used while in other embodiments, thicker layers may be used. In some embodiments structures having features positioned with micron level precision and minimum features size on the order of tens of microns are to be formed. In other embodiments structures with less precise feature placement and/or larger minimum features may be formed. In still other embodiments, higher precision and smaller minimum feature sizes may be desirable. In the present application meso-scale and millimeter scale have the same meaning and refer to devices that may have one or more dimensions extending into the 0.5-20 millimeter range, or somewhat larger and with features positioned with precision in the 10-100 micron range and with minimum features sizes on the order of 100 microns.

The various embodiments, alternatives, and techniques disclosed herein may form multi-layer structures using a single patterning technique on all layers or using different patterning techniques on different layers. For example, Various embodiments of the invention may perform selective patterning operations using conformable contact masks and masking operations (i.e. operations that use masks which are contacted to but not adhered to a substrate), proximity masks and masking operations (i.e. operations that use masks that at least partially selectively shield a substrate by their proximity to the substrate even if contact is not made), non-conformable masks and masking operations (i.e. masks and operations based on masks whose contact surfaces are not significantly conformable), and/or adhered masks and masking operations (masks and operations that use masks that are adhered to a substrate onto which selective deposition or etching is to occur as opposed to only being contacted to it). Conformable contact masks, proximity masks, and non-conformable contact masks share the property that they are preformed and brought to, or in proximity to, a surface which is to be treated (i.e. the exposed portions of the surface are to be treated). These masks can generally be removed without damaging the mask or the surface that received treatment to which they were contacted, or located in proximity to. Adhered masks are generally formed on the surface to be treated (i.e. the portion of that surface that is to be masked) and bonded to that surface such that they cannot be separated from that surface without being completely destroyed damaged beyond any point of reuse. Adhered masks may be formed in a number of ways including (1) by application of a photoresist, selective exposure of the photoresist, and then development of the photoresist, (2) selective transfer of pre-patterned masking material, and/or (3) direct formation of masks from computer controlled depositions of material.

Patterning operations may be used in selectively depositing material and/or may be used in the selective etching of material. Selectively etched regions may be selectively filled in or filled in via blanket deposition, or the like, with a different desired material. In some embodiments, the layer-by-layer build up may involve the simultaneous formation of portions of multiple layers. In some embodiments, depositions made in association with some layer levels may result in depositions to regions associated with other layer levels (i.e. regions that lie within the top and bottom boundary levels that define a different layer's geometric configuration). Such use of selective etching and interlaced material deposition in association with multiple layers is described in U.S. Pat. No. 7,252,861, which is hereby incorporated herein by reference as if set forth in full.

Temporary substrates on which structures may be formed may be of the sacrificial-type (i.e. destroyed or damaged during separation of deposited materials to the extent they cannot be reused), non-sacrificial-type (i.e. not destroyed or excessively damaged, i.e. not damaged to the extent they may not be reused, e.g. with a sacrificial or release layer located between the substrate and the initial layers of a structure that is formed). Non-sacrificial substrates may be considered reusable, with little or no rework (e.g. replanarizing one or more selected surfaces or applying a release layer, and the like) though they may or may not be reused for a variety of reasons.

### Definitions

This section of the specification is intended to set forth definitions for a number of specific terms that may be useful in describing the subject matter of the various embodiments of the invention. It is believed that the meanings of most if not all of these terms is clear from their general use in the specification but they are set forth hereinafter to remove any ambiguity that may exist. It is intended that these definitions be used in understanding the scope and limits of any claims that use these specific terms. As far as interpretation of the claims of this patent disclosure are concerned, it is intended that these definitions take presence over any contradictory definitions or allusions found in any materials which are incorporated herein by reference.

“Build” as used herein refers, as a verb, to the process of building a desired structure or plurality of structures from a plurality of applied or deposited materials which are stacked and adhered upon application or deposition or, as a noun, to the physical structure or structures formed from such a process. Depending on the context in which the term is used, such physical structures may include a desired structure embedded within a sacrificial material or may include only desired physical structures which may be separated from one another or may require dicing and/or slicing to cause separation.

“Build axis” or “build orientation” is the axis or orientation that is substantially perpendicular to substantially planar levels of deposited or applied materials that are used in building up a structure. The planar levels of deposited or applied materials may be or may not be completely planar but are substantially so in that the overall extent of their cross-sectional dimensions are significantly greater than the height of any individual deposit or application of material (e.g. 100, 500, 1000, 5000, or more times greater). The planar nature of the deposited or applied materials may come about from use of a process that leads to planar deposits or it may result from a planarization process (e.g. a process that includes mechanical abrasion, e.g. lapping, fly cutting, grinding, or the like) that is used to remove material regions of excess height. Unless explicitly noted otherwise, “vertical” as used herein refers to the build axis or nominal build axis (if the layers are not stacking with perfect registration) while “horizontal” refers to a direction within the plane of the layers (i.e. the plane that is substantially perpendicular to the build axis).

“Build layer” or “layer of structure” as used herein does not refer to a deposit of a specific material but instead refers to a region of a build located between a lower boundary level and an upper boundary level which generally defines a single cross-section of a structure being formed or structures which are being formed in parallel. Depending on the details of the actual process used to form the structure, build layers are generally formed on and adhered to previously formed build layers. In some processes the boundaries between build layers are defined by planarization operations which result in successive build layers being formed on substantially planar upper surfaces of previously formed build layers. In some embodiments, the substantially planar upper surface of the preceding build layer may be textured to improve adhesion between the layers. In other build processes, openings may exist in or be formed in the upper surface of a previous but only partially formed build layers such that the openings in the previous build layers are filled with materials deposited in association with current build layers which will cause interlacing of build layers and material deposits. Such interlacing is described in U.S. patent application Ser. No. 10/434,519 now U.S. Pat. No. 7,252,861. This referenced application is incorporated herein by reference as if set forth in full. In most embodiments, a build layer includes at least one primary structural material and at least one primary sacrificial material. However, in some embodiments, two or more primary structural materials may be used without a primary sacrificial material (e.g. when one primary structural material is a dielectric and the other is a conductive material). In some embodiments, build layers are distinguishable from each other by the source of the data that is used to yield patterns of the deposits, applications, and/or etchings of material that form the respective build layers. For example, data descriptive of a structure to be formed which is derived from data extracted from different vertical levels of a data representation of the structure define different build layers of the structure. The vertical separation of successive pairs of such descriptive data may define the thickness of build layers associated with the data. As used herein, at times, “build layer” may be loosely referred simply as “layer”. In many embodiments, deposition thickness of primary structural or sacrificial materials (i.e. the thickness of any particular material after it is deposited) is generally greater than the layer thickness and a net deposit thickness is set via one or more planarization processes which may include, for example, mechanical abrasion (e.g. lapping, fly cutting, polishing, and the like) and/or chemical etching (e.g. using selective or non-selective etchants). The lower boundary and upper boundary for a build layer may be set and defined in different ways. From a design point of view they may be set based on a desired vertical resolution of the structure (which may vary with height). From a data manipulation point of view, the vertical layer boundaries may be defined as the vertical levels at which data descriptive of the structure is processed or the layer thickness may be defined as the height separating successive levels of cross-sectional data that dictate how the structure will be formed. From a fabrication point of view, depending on the exact fabrication process used, the upper and lower layer boundaries may be defined in a variety of different ways. For example by planarization levels or effective planarization levels (e.g. lapping levels, fly cutting levels, chemical mechanical polishing levels, mechanical polishing levels, vertical positions of structural and/or sacrificial materials after relatively uniform etch back following a mechanical or chemical mechanical planarization process). For example, by levels at which process steps or operations are repeated. At levels at which, at least theoretically, lateral extends of structural material can be changed to define new cross-sectional features of a structure.

“Layer thickness” is the height along the build axis between a lower boundary of a build layer and an upper boundary of that build layer.

“Planarization” is a process that tends to remove materials, above a desired plane, in a substantially non-selective manner such that all deposited materials are brought to a substantially common height or desired level (e.g. within 20%, 10%, 5%, or even 1% of a desired layer boundary level). For example, lapping removes material in a substantially non-selective manner though some amount of recession one material or another may occur (e.g. copper may recess relative to nickel). Planarization may occur primarily via mechanical means, e.g. lapping, grinding, fly cutting, milling, sanding, abrasive polishing, frictionally induced melting, other machining operations, or the like (i.e. mechanical planarization). Mechanical planarization maybe followed or proceeded by thermally induced planarization (e.g. melting) or chemically induced planarization (e.g. etching). Planarization may occur primarily via a chemical and/or electrical means (e.g. chemical etching, electrochemical etching, or the like). Planarization may occur via a simultaneous combination of mechanical and chemical etching (e.g. chemical mechanical polishing (CMP)).

“Structural material” as used herein refers to a material that remains part of the structure when put into use.

“Supplemental structural material” as used herein refers to a material that forms part of the structure when the structure is put to use but is not added as part of the build layers but instead is added to a plurality of layers simultaneously (e.g. via one or more coating operations that applies the material, selectively or in a blanket fashion, to a one or more surfaces of a desired build structure that has been released from a sacrificial material.

“Primary structural material” as used herein is a structural material that forms part of a given build layer and which is typically deposited or applied during the formation of that build layer and which makes up more than 20% of the structural material volume of the given build layer. In some embodiments, the primary structural material may be the same on each of a plurality of build layers or it may be different on different build layers. In some embodiments, a given primary structural material may be formed from two or more materials by the alloying or diffusion of two or more materials to form a single material.

“Secondary structural material” as used herein is a structural material that forms part of a given build layer and is typically deposited or applied during the formation of the given build layer but is not a primary structural material as it individually accounts for only a small volume of the structural material associated with the given layer. A secondary structural material will account for less than 20% of the volume of the structural material associated with the given layer. In some preferred embodiments, each secondary structural material may account for less than 10%, 5%, or even 2% of the volume of the structural material associated with the given layer. Examples of secondary structural materials may include seed layer materials, adhesion layer materials, barrier layer materials (e.g. diffusion barrier material), and the like. These secondary structural materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931, now U.S. Pat. No. 7,239,219. In other embodiments, such coatings may be applied in a non-planar manner, for example, in openings in and over a patterned masking material that has been applied to previously planarized layers of material as taught in U.S. patent application Ser. No. 10/841,383, now U.S. Pat. No. 7,195,989. These referenced applications are incorporated herein by reference as if set forth in full herein.

“Functional structural material” as used herein is a structural material that would have been removed as a sacrificial material but for its actual or effective encapsulation by other structural materials. Effective encapsulation refers, for example, to the inability of an etchant to attack the functional structural material due to inaccessibility that results from a very small area of exposure and/or due to an elongated or tortuous exposure path. For example, large (10,000 μm2) but thin (e.g. less than 0.5 microns) regions of sacrificial copper sandwiched between deposits of nickel may define regions of functional structural material depending on ability of a release etchant to remove the sandwiched copper.

“Sacrificial material” is material that forms part of a build layer but is not a structural material. Sacrificial material on a given build layer is separated from structural material on that build layer after formation of that build layer is completed and more generally is removed from a plurality of layers after completion of the formation of the plurality of layers during a “release” process that removes the bulk of the sacrificial material or materials. In general sacrificial material is located on a build layer during the formation of one, two, or more subsequent build layers and is thereafter removed in a manner that does not lead to a planarized surface. Materials that are applied primarily for masking purposes, i.e. to allow subsequent selective deposition or etching of a material, e.g. photoresist that is used in forming a build layer but does not form part of the build layer) or that exist as part of a build for less than one or two complete build layer formation cycles are not considered sacrificial materials as the term is used herein but instead shall be referred as masking materials or as temporary materials. These separation processes are sometimes referred to as a release process and may or may not involve the separation of structural material from a build substrate. In many embodiments, sacrificial material within a given build layer is not removed until all build layers making up the three-dimensional structure have been formed. Of course sacrificial material may be, and typically is, removed from above the upper level of a current build layer during planarization operations during the formation of the current build layer. Sacrificial material is typically removed via a chemical etching operation but in some embodiments may be removed via a melting operation or electrochemical etching operation. In typical structures, the removal of the sacrificial material (i.e. release of the structural material from the sacrificial material) does not result in planarized surfaces but instead results in surfaces that are dictated by the boundaries of structural materials located on each build layer. Sacrificial materials are typically distinct from structural materials by having different properties therefrom (e.g. chemical etchability, hardness, melting point, etc.) but in some cases, as noted previously, what would have been a sacrificial material may become a structural material by its actual or effective encapsulation by other structural materials. Similarly, structural materials may be used to form sacrificial structures that are separated from a desired structure during a release process via the sacrificial structures being only attached to sacrificial material or potentially by dissolution of the sacrificial structures themselves using a process that is insufficient to reach structural material that is intended to form part of a desired structure. It should be understood that in some embodiments, small amounts of structural material may be removed, after or during release of sacrificial material. Such small amounts of structural material may have been inadvertently formed due to imperfections in the fabrication process or may result from the proper application of the process but may result in features that are less than optimal (e.g. layers with stairs steps in regions where smooth sloped surfaces are desired. In such cases the volume of structural material removed is typically minuscule compared to the amount that is retained and thus such removal is ignored when labeling materials as sacrificial or structural. Sacrificial materials are typically removed by a dissolution process, or the like, that destroys the geometric configuration of the sacrificial material as it existed on the build layers. In many embodiments, the sacrificial material is a conductive material such as a metal. As will be discussed hereafter, masking materials though typically sacrificial in nature are not termed sacrificial materials herein unless they meet the required definition of sacrificial material.

“Supplemental sacrificial material” as used herein refers to a material that does not form part of the structure when the structure is put to use and is not added as part of the build layers but instead is added to a plurality of layers simultaneously (e.g. via one or more coating operations that applies the material, selectively or in a blanket fashion, to a one or more surfaces of a desired build structure that has been released from an initial sacrificial material. This supplemental sacrificial material will remain in place for a period of time and/or during the performance of certain post layer formation operations, e.g. to protect the structure that was released from a primary sacrificial material, but will be removed prior to putting the structure to use.

“Primary sacrificial material” as used herein is a sacrificial material that is located on a given build layer and which is typically deposited or applied during the formation of that build layer and which makes up more than 20% of the sacrificial material volume of the given build layer. In some embodiments, the primary sacrificial material may be the same on each of a plurality of build layers or may be different on different build layers. In some embodiments, a given primary sacrificial material may be formed from two or more materials by the alloying or diffusion of two or more materials to form a single material.

“Secondary sacrificial material” as used herein is a sacrificial material that is located on a given build layer and is typically deposited or applied during the formation of the build layer but is not a primary sacrificial materials as it individually accounts for only a small volume of the sacrificial material associated with the given layer. A secondary sacrificial material will account for less than 20% of the volume of the sacrificial material associated with the given layer. In some preferred embodiments, each secondary sacrificial material may account for less than 10%, 5%, or even 2% of the volume of the sacrificial material associated with the given layer. Examples of secondary structural materials may include seed layer materials, adhesion layer materials, barrier layer materials (e.g. diffusion barrier material), and the like. These secondary sacrificial materials are typically applied to form coatings having thicknesses less than 2 microns, 1 micron, 0.5 microns, or even 0.2 microns). The coatings may be applied in a conformal or directional manner (e.g. via CVD, PVD, electroless deposition, or the like). Such coatings may be applied in a blanket manner or in a selective manner. Such coatings may be applied in a planar manner (e.g. over previously planarized layers of material) as taught in U.S. patent application Ser. No. 10/607,931, now U.S. Pat. No. 7,239,219. In other embodiments, such coatings may be applied in a non-planar manner, for example, in openings in and over a patterned masking material that has been applied to previously planarized layers of material as taught in U.S. patent application Ser. No. 10/841,383, now U.S. Pat. No. 7,195,989. These referenced applications are incorporated herein by reference as if set forth in full herein.

“Adhesion layer”, “seed layer”, “barrier layer”, and the like refer to coatings of material that are thin in comparison to the layer thickness and thus generally form secondary structural material portions or sacrificial material portions of some layers. Such coatings may be applied uniformly over a previously formed build layer, they may be applied over a portion of a previously formed build layer and over patterned structural or sacrificial material existing on a current (i.e. partially formed) build layer so that a non-planar seed layer results, or they may be selectively applied to only certain locations on a previously formed build layer. In the event such coatings are non-selectively applied, selected portions may be removed (1) prior to depositing either a sacrificial material or structural material as part of a current layer or (2) prior to beginning formation of the next layer or they may remain in place through the layer build up process and then etched away after formation of a plurality of build layers.

“Masking material” is a material that may be used as a tool in the process of forming a build layer but does not form part of that build layer. Masking material is typically a photopolymer or photoresist material or other material that may be readily patterned. Masking material is typically a dielectric. Masking material, though typically sacrificial in nature, is not a sacrificial material as the term is used herein. Masking material is typically applied to a surface during the formation of a build layer for the purpose of allowing selective deposition, etching, or other treatment and is removed either during the process of forming that build layer or immediately after the formation of that build layer.

“Multilayer structures” are structures formed from multiple build layers of deposited or applied materials.

“Multilayer three-dimensional (or 3D or 3-D) structures” are Multilayer Structures that meet at least one of two criteria: (1) the structural material portion of at least two layers of which one has structural material portions that do not overlap structural material portions of the other.

“Complex multilayer three-dimensional (or 3D or 3-D) structures” are multilayer three-dimensional structures formed from at least three layers where a line may be defined that hypothetically extends vertically through at least some portion of the build layers of the structure will extend from structural material through sacrificial material and back through structural material or will extend from sacrificial material through structural material and back through sacrificial material (these might be termed vertically complex multilayer three-dimensional structures). Alternatively, complex multilayer three-dimensional structures may be defined as multilayer three-dimensional structures formed from at least two layers where a line may be defined that hypothetically extends horizontally through at least some portion of a build layer of the structure that will extend from structural material through sacrificial material and back through structural material or will extend from sacrificial material through structural material and back through sacrificial material (these might be termed horizontally complex multilayer three-dimensional structures). Worded another way, in complex multilayer three-dimensional structures, a vertically or horizontally extending hypothetical line will extend from one or structural material or void (when the sacrificial material is removed) to the other of void or structural material and then back to structural material or void as the line is traversed along at least a portion of the line.

“Moderately complex multilayer three-dimensional (or 3D or 3-D) structures” are complex multilayer 3D structures for which the alternating of void and structure or structure and void not only exists along one of a vertically or horizontally extending line but along lines extending both vertically and horizontally.

“Highly complex multilayer (or 3D or 3-D) structures” are complex multilayer 3D structures for which the structure-to-void-to-structure or void-to-structure-to-void alternating occurs once along the line but occurs a plurality of times along a definable horizontally or vertically extending line.

“Up-facing feature” is an element dictated by the cross-sectional data for a given build layer “n” and a next build layer “n+1” that is to be formed from a given material that exists on the build layer “n” but does not exist on the immediately succeeding build layer “n+1”. For convenience the term “up-facing feature” will apply to such features regardless of the build orientation.

“Down-facing feature” is an element dictated by the cross-sectional data for a given build layer “n” and a preceding build layer “n−1” that is to be formed from a given material that exists on build layer “n” but does not exist on the immediately preceding build layer “n−1”. As with up-facing features, the term “down-facing feature” shall apply to such features regardless of the actual build orientation.

“Continuing region” is the portion of a given build layer “n” that is dictated by the cross-sectional data for the given build layer “n”, a next build layer “n+1” and a preceding build layer “n−1” that is neither up-facing nor down-facing for the build layer “n”.

“Minimum feature size” refers to a necessary or desirable spacing between structural material elements on a given layer that are to remain distinct in the final device configuration. If the minimum feature size is not maintained on a given layer, the fabrication process may result in structural material inadvertently bridging the two structural elements due to masking material failure or failure to appropriately fill voids with sacrificial material during formation of the given layer such that during formation of a subsequent layer structural material inadvertently fills the void. More care during fabrication can lead to a reduction in minimum feature size or a willingness to accept greater losses in productivity can result in a decrease in the minimum feature size. However, during fabrication for a given set of process parameters, inspection diligence, and yield (successful level of production) a minimum design feature size is set in one way or another. The above described minimum feature size may more appropriately be termed minimum feature size of sacrificial material regions. Conversely a minimum feature size for structure material regions (minimum width or length of structural material elements) may be specified. Depending on the fabrication method and order of deposition of structural material and sacrificial material, the two types of minimum feature sizes may be different. In practice, for example, using electrochemical fabrication methods and described herein, the minimum features size on a given layer may be roughly set to a value that approximates the layer thickness used to form the layer and it may be considered the same for both structural and sacrificial material widths and lengths. In some more rigorously implemented processes, examination regiments, and rework requirements, it may be set to an amount that is 80%, 50%, or even 30% of the layer thickness. Other values or methods of setting minimum feature sizes may be set.

“Sublayer” as may be used herein refers to a portion of a build layer that typically includes the full lateral extents of that build layer but only a portion of its height. A sublayer is usually a vertical portion of build layer that undergoes independent processing compared to another sublayer of that build layer.

Cylindrical Cutting Devices

Various cylindrical cutting devices or instrument embodiments will be discussed below. These devices may be used in a number of different tissue removal methods, such as planing, coring, milling, or drilling. Such tissue removal methods can be used in various applications including: (1) Disc, other tissue, or bone in the spinal region, for example, to relieve pressure on spinal nerves, (2) Ear, nose (sinus), and throat surgery, (3) ophthalmic procedures such as cataract surgery; (4) Cardiovascular (can be delivered over a guide wire) surgery or procedures such as (a) Blood clot removal (Thrombectomy); (b) Chronic total occlusion (CTO); (c) Atherectomy; (d) Removal of heart tissue; (5) Neurovascular procedures such as thrombectomy; (6) Breast surgeries or procedures such as (a) Breast duct papilloma, and (b) Lumpectomy; (7) Orthopedic surgeries and procedures such as (a) Joint surgeries; (b) Removal of bone spurs; and (c) Arthroscopic surgeries; (8) Peripheral artery disease surgeries and procedures; (9) other thrombectomy and atherectomy procedures and (10) Removal of tumors, cancerous tissue, and other excess tissue masses. The devices of various embodiments of the invention may also be used in non-medical applications.

The cutting devices described herein can advantageously be constructed using the electrochemical fabrication process. Using the electrochemical fabrication process allows the devices to be on the micrometer or nanometer scale and have precision on the order of tens of microns. Medical devices having such scale and precision are advantageous over conventional medical devices because they can be sharper, have more cutting surfaces, and be more intricately shaped. As a result, the medical devices described herein can be used for selective and accurate removal of tissue or other material within the body.

Further, using the electrochemical fabrication process is advantageous because the scale and precision available by doing so allows the medical devices to be configured to be used in conjunction with additional therapeutic or diagnostic elements. For example, the medical devices described herein may be used in conjunction with ancillary components extending through the center of the device, such as guide wires, endoscopes or other imaging methods (IVUS, OCT, OFDI, etc.), aspiration, irrigation, and other micro-scale or millimeter-scale devices and instruments such as distal protection devices (see U.S. patent application Ser. No. 12/179,573), positioning instruments such as expanders (see U.S. patent application Ser. No. 12/179,573), other tissue shredding devices such as those described in U.S. patent application Ser. No. 12/490,301, and guiding and configurable elements such as those described in U.S. patent application Ser. Nos. 12/169,528; 12/179,295; and Ser. No. 12/144,618.

Although the medical devices described herein can be produced using the electrochemical fabrication process, additional fabrication processes may also be used. The cutting devices described herein can each include two concentric components, which can be configured to rotate relative to one another to perform the desired surgical function. As such, only one concentric component can be rotated, both can be rotated in opposite directions, or both can be rotated in the same direction, but at different rates. The dimensions of the various cutting devices can be adjusted to obtain a desired degree of tissue removal.

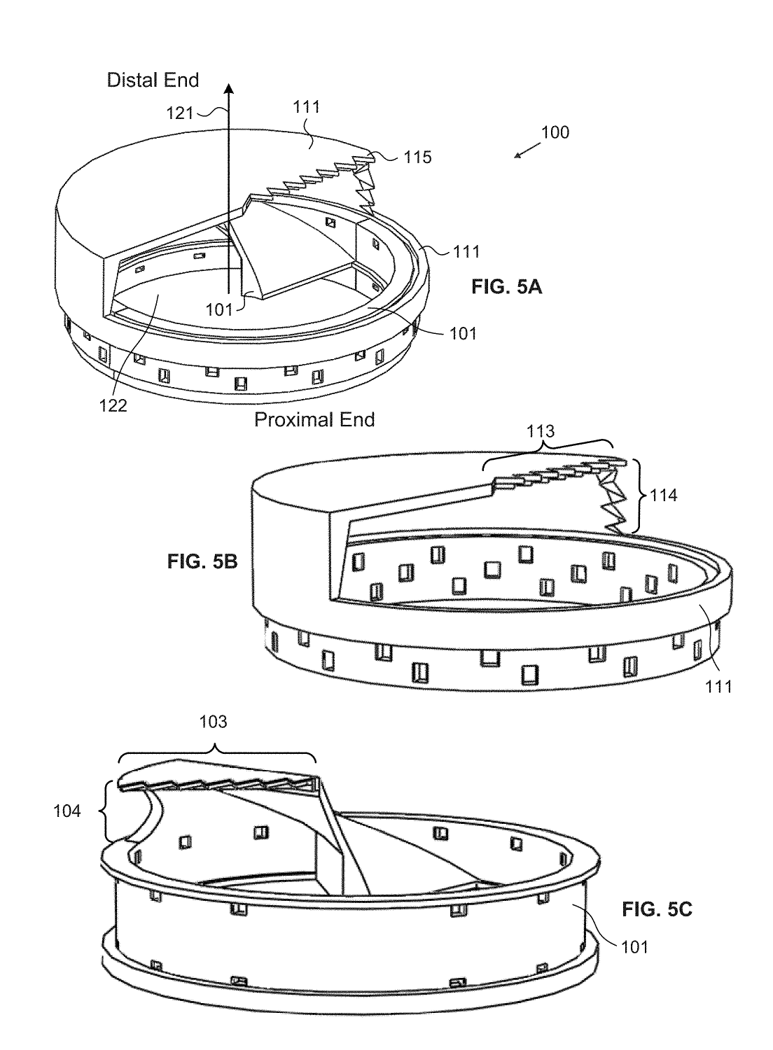

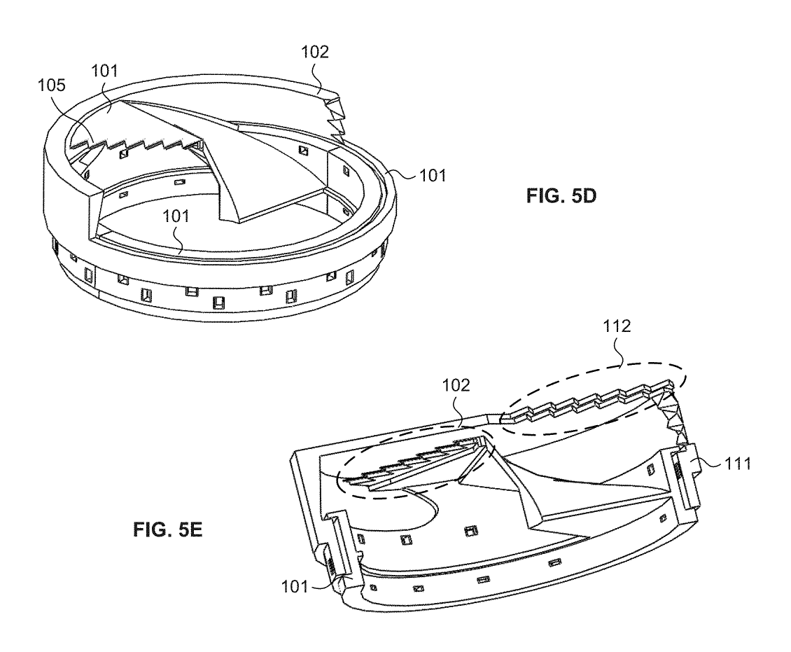

FIGS. 5A-5E illustrate a cutting device 100. The first component 101 of the device 100 includes an inner cutting element 102, having a primary cutting surface 103 and a secondary cutting surface 104. The second component 111 likewise includes an outer cutting element 112, having a primary surface 113 and a secondary surface 114. When assembled as shown in FIG. 5E, the first component 101 is capable of rotating relative to the second component 111, and the interaction of the cutting surfaces can cause shearing away of layers of tissue or other material as the inner and outer cutting elements rotate past one another. In some embodiments, either or both components can be configured to rotate in either direction. For example, the first component can rotate counter-clockwise, and the second component can rotate clockwise.

Both the inner and outer cutters 102, 112 can have singled-sided substantially radially-extending primary cutting surfaces 103 and 113 as well as secondary cutting surfaces 104 and 114 extending substantially axially at the radial extremes of the components. Having a primary cutting surface extending substantially radially and a secondary cutting surface extending substantially axially can be advantageous over prior art cutting devices because it provides more cutting surfaces for shearing off tissue. Further, the axial length of the secondary cutting surfaces 104 and 114 can be less than 100 microns, such as less than 50 microns, such as less than 10 microns. Such a small axial length allows for accurate removal of small layers of tissue, such as layers between 2 and 5 microns thick. Thus, for example, cutting device 100 can be used for planing of thin slices of tissue.

Teeth 105 and 115 can extend along the primary and/or the secondary cutting surfaces. The teeth 105 and 115 can all be configured such that they extend radially. The teeth 105 and 115 can be configured to, upon rotation of the first and second components 101, 111, engage one another substantially point-to-point, relative to a centered longitudinal axis 121 of relative rotation of the elements. The teeth 105 and 115 can aid in shearing off layers of tissue during the planing process.

As shown in the figures, the cutting surface of the first component 101 is supported by a sloped surface that sweeps a three-dimensional curve (i.e. sweeping in radial, axial or longitudinal, and azimuthal directions). The proximal facing portion of this sloped surface, when rotating in a counterclockwise direction, may aid in pushing sheared off material in a proximal and longitudinal direction to help remove material and ensure that the cutting surface of the blade is cleared of material and ready to shear off newly encountered material.

As shown, the first component 101 of device 100 includes an intake window 122. The intake window 122 can, for example, extend across at least one-half of the distal end of the cutting device, such as approximately one-half of the distal end of the cutting device. The intake window 122 permits tissue to extend into the interior of cutting device 100 to enable the interacting cutting surfaces of the first and second components to shear the tissue, for example during a planing process.

As shown in the figures the components may be formed with a plurality of etching or release holes so that the individual components may be formed using a multi-material, multi-layer fabrication method and then the sacrificial material readily removed. In alternative embodiments, fewer, more, or even no release holes may be formed. In some embodiments, the components may be formed using an interference bushings, or with intermediate bearing elements, for example, to provide smoother operation or tighter formation tolerances. In some alternative embodiments fluid flow paths and outlets may exist between the components and may receive a fluid during operation of the device so as to provide a fluid bearing for improved device operation.

The device 100 can be a micro-scale device. Thus, the diameter of the device 100 can be less than 5 mm, such as less than 3 mm, e.g. less than 1 mm. The minimum feature size can be on the order of tens of microns, i.e. less than 100 microns, such as less than 50 microns. Moreover, the precision of the device build can be on the micron level, i.e. between 1 and 10 microns. Having a micro-scale device can advantageously allow the device to be used in small areas of the body that are unreachable by larger devices. Moreover, the precision of the build and the minimum feature size can be useful for very precise and specific tissue removal, such as planing of tissue layers of only a few microns thick. These micro-scale devices may be made using the electrochemical fabrication process described above.

Various alternatives to this embodiment are possible and include, for example, alternative blade configurations and intake configurations. For example, the teeth of the cutting elements may be made to encounter one another other than in a tip-to-tip configuration, the teeth may be removed in favor of straight blades, and the cutting blades may have cutting surfaces that have lengths which extend not just radially but also have an azimuthal component of length as well. In some embodiments, the cutting elements may be provided with cutting surfaces to allow cutting in either direction of rotation. In some alternative embodiments, the intake opening, which is defined by the distal cap of component 111 may be made larger by decreasing the azimuthal sweep or extent of the cap or smaller by increasing the azimuthal extent of the cap. In some embodiments, different numbers of inner cutting elements may form part of the inner component (e.g. 1, 2, 3, 4, or more cutting elements), and different numbers of outer cutting elements may form part of the outer component, and in some embodiments, these numbers of inner and outer cutting elements need not match. In some embodiments, cutting elements may be contained on a single component, two components, or more than two components.

During use, the two components 101, 111 of this working end of the cutting device may have their proximal ends joined or otherwise coupled to tubes or other rotatable elements such that one component (i.e. each including its respective cutting elements) stays stationary while the other rotates, such the two components 101, 111 rotate in opposite directions, or such that the two components 101, 111 rotate in the same direction but at different rates such that they still move past one another to provide shearing. During some uses, the components 101, 111 may be made to periodically, or possibly upon input from sensors (e.g. an input indicating a stall or excess slowing of the rotation), rotate a partial rotation in reverse to provide an opportunity for additional shearing attempts. During some uses, the cutting may be accompanied by aspiration from distal to proximal end to provide enhanced transport of sheared off material. In some embodiments, aspiration may be accompanied by appropriately directed irrigation. In some embodiments, more proximally located cutting and/or transport elements can be included on the components 101, 111 to cause further maceration of the removed material or proximal transportation of the material.

In some alternative embodiments, the rotation of one or both of the concentric components may occur via one or more rotating tubes that may be located within a catheter. The tubes may be driven by rotational driving elements located at a significant distance from the working area that is being operated on (e.g. outside the body of a patient). In other embodiments, the rotating tubes or other elements may be driven by a fluid driven turbine (e.g. driven by an irrigation fluid of other fluid) that is located within the catheter or other instrumental lumen.

In some embodiments, the instrument components shown in FIGS. 5A-5E may be formed using one of the multi-layer multi-material fabrication processes set forth herein or incorporated herein by reference. In some embodiments, one of the components, or part the components may be made by one of these multi-layer multi-material fabrication process while the other component or component portions may be made by one or more different processes. In still other embodiments, both of the components may be made by processes other than multi-layer, multi-material fabrication process. For example, one or both components, or portions thereof may be made from a tube which is cut to a desired shape and then bent to a desired configuration and perhaps with portions welded or otherwise joined to maintain the created configuration.

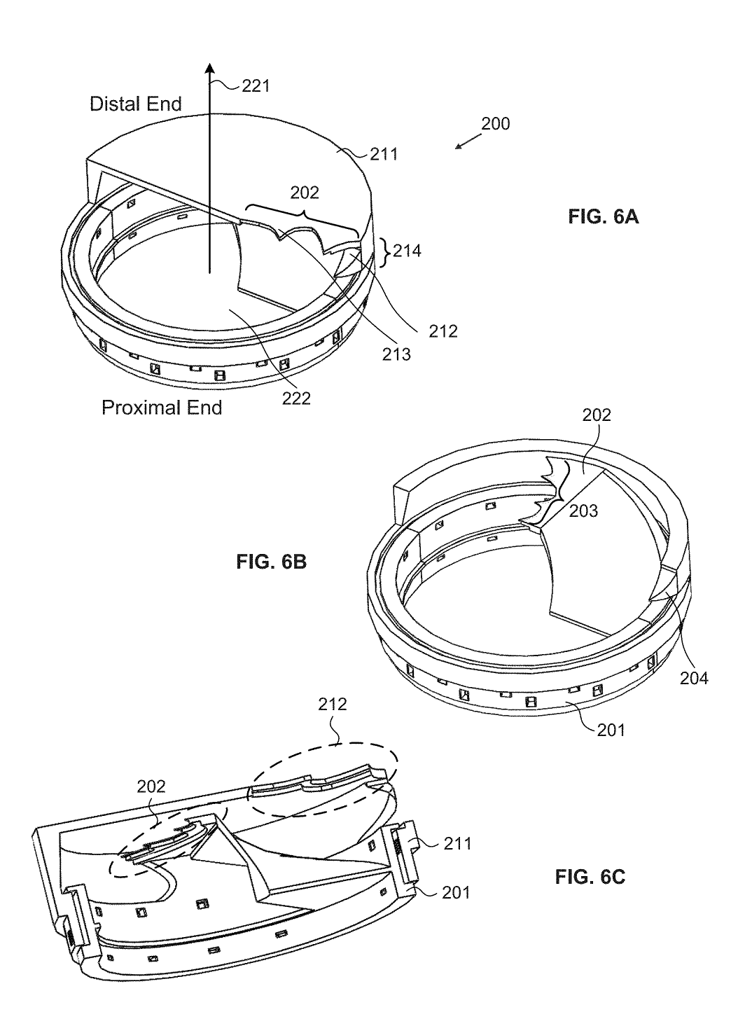

FIGS. 6A-6C provide various views of a working end of a cutting device 200. The cutting device 200 includes first and second components 201 and 211. The first component 201 includes an inner cutting element 202, having a primary cutting surface 203 and a secondary cutting surface 204. The second component 211 includes an outer cutting element 212, having a primary surface 213 and a secondary surface 214. One or both of the first and second components 201, 211 is capable of rotating about a central longitudinal axis 221 to cause relative rotation with respect to one another. The interaction of cutting surfaces 203 and 204 with cutting surfaces 213 and 214, respectively, during such relative rotation can cause shearing away of tissue or other material as the inner and outer cutting elements rotate past one another.

Both the inner and outer cutters 202, 212 can have singled-sided radial extending, and slightly azimuthal extending, primary cutting surfaces 203 and 213 as well as secondary cutting surfaces 204 and 214 extending axially at the radial extremes of the components. Teeth 205 and 215 can extend radially along the primary and/or secondary cutting surfaces.

An intake window, such as an intake window 222 of the device exists on one-half of the distal end of the cutting device 200. The intake window 222 permits tissue to extend into the interior of cutting device 200 to enable the interacting cutting surfaces of the first and second components to shear the tissue, for example during a planing process. Further, a distal cap of element 211 is located on the other half of the distal end of the cutting device 200. The cutter 200 can have many of the same advantages of the cutter 100. For example, the cutter 100 can be a micro-scale device and can have thin axially-extending cutting surfaces, allowing for access to small areas and specific and precise removal of very small layers of tissue, such as during a planing process.

Numerous variations of the cutting device 200 exist, some of which are similar, mutatis mutandis, to those noted above with regard to the first embodiment.

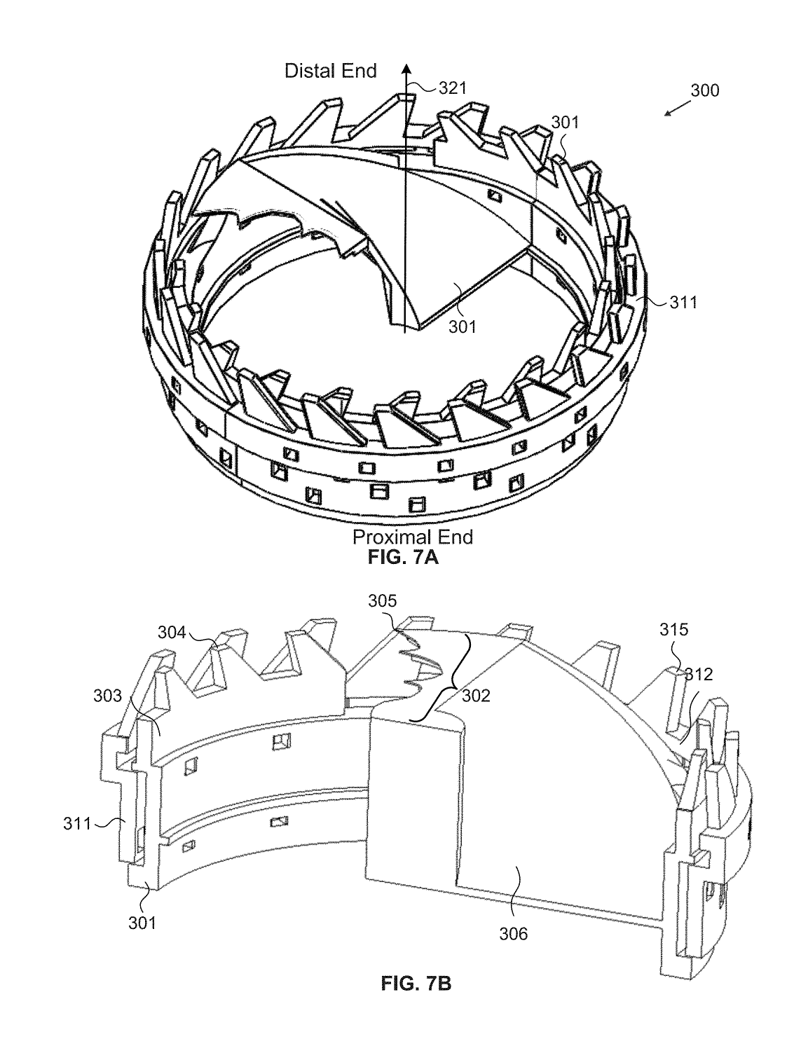

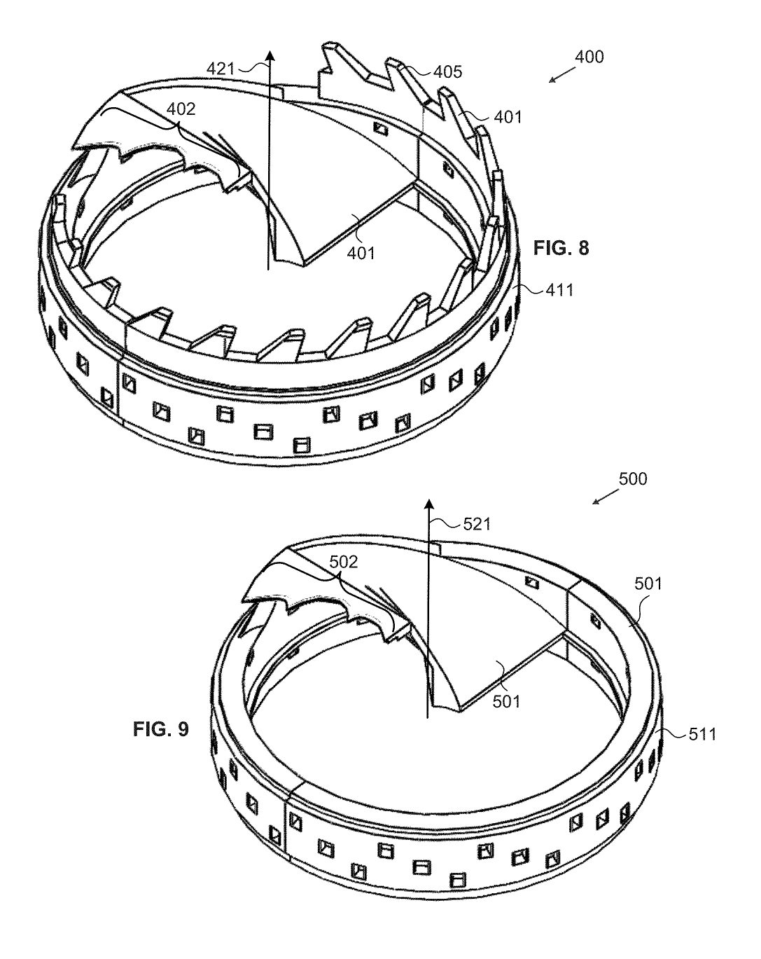

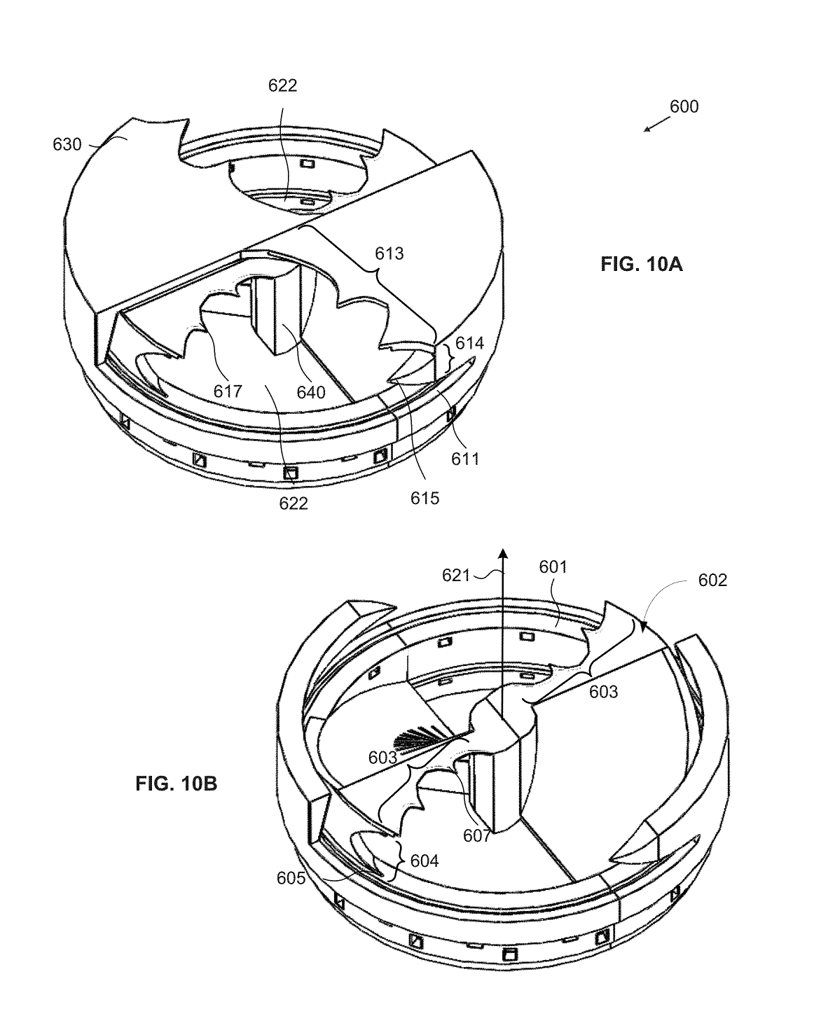

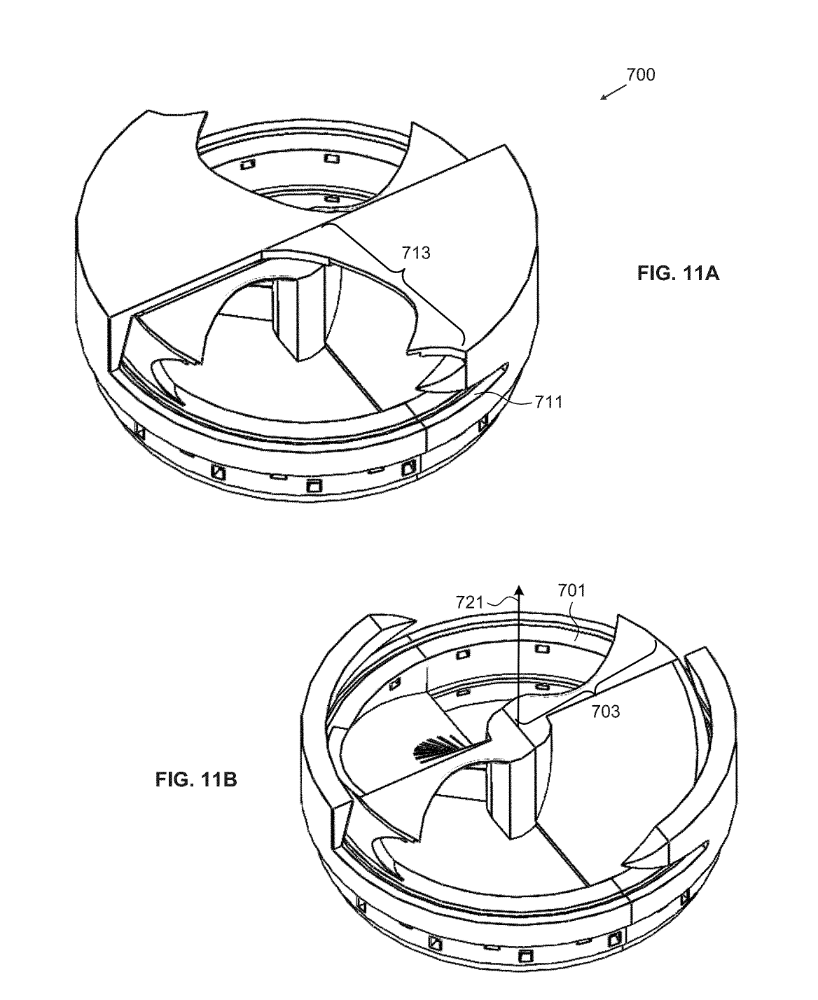

FIGS. 7A-7B provide perspective and cut views of a working end of a cutting device 300. The first component 301 of the device 300 includes an inner cutting element 302 having optional teeth 305 that extend perpendicular to the axis of rotation and a secondary peripheral cutting surface 303 with axially-extending teeth 305. The second component 311 is disposed radially outward from first element 301 and includes an outer cutting element 312 with axially-extending teeth 315. The first component 301 is capable of rotating with respect to the second component 311, which causes shearing at the periphery due to the interaction of cutting surfaces 303 and 312 while the cutting surface 302 cuts a plane of tissue. Sloped surface 306 helps draw material from the distal end of the device toward the proximal end. In some embodiments, the first component can also be configured to rotate. For example, the first component can rotate counter-clockwise, and the second component can rotate clockwise.

Both cutting surfaces 304 and 312 are provided with teeth in a crown configuration, i.e. both have teeth extending axially. The teeth can be used to drive into tissue. The teeth can have a maximum radial thickness of less than 50 microns, such as approximately 30 microns. Further, the teeth can have a pitch of less than 200 microns, such as less than 100 microns. The device 300 can be used for coring and slicing a substantially circular plane of tissue, i.e. for conducting a biopsy. The small teeth of the cutting device 300 can allow for removal of very small tissue samples, such as samples that are less than 5 microns, such as between 2 and 5 microns. Removing such small samples avoids excessive damage to surrounding tissue.

The intake window 322 of the device 300 covers nearly the entire 360 degree azimuthal region of the components to allow tissue to extend proximally into the device for shearing and easy removal of the tissue sample for analysis. The device 300 can be a micro-scale device, allowing it access to otherwise inaccessible areas of the body and may be made using the electrochemical fabrication process described above.

Numerous variations the cutting device 300 exist, some of which are similar, mutatis mutandis, to those noted above with regard to cutting device 100.

FIGS. 8, 9, 13, and 16 show components of devices similar to device 300, i.e., that include axially-extending teeth. Thus, the devices can include many of the same features and advantages as device 300.

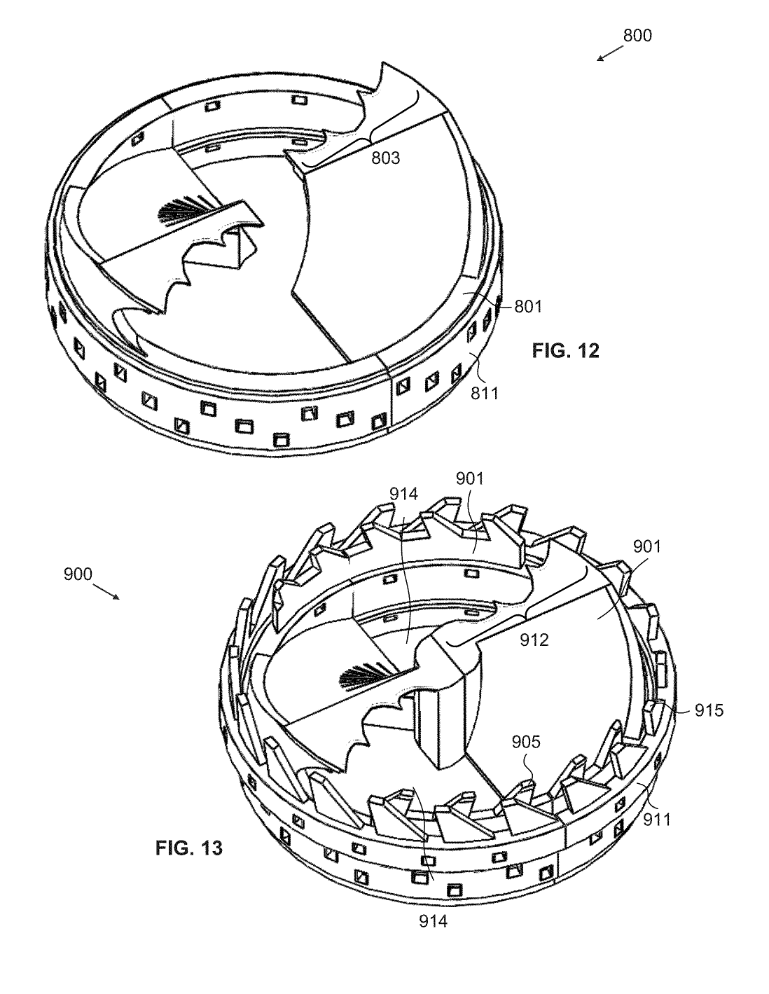

FIGS. 8 and 9 are similar to the cutting device 300 with the exception that the cutter 400 (FIG. 8) has the outer crown cutting teeth removed while the cutter 500 has both the inner and out crown cutting teeth removed.

FIG. 13 provides a perspective view of a working end of a cutting device 900 having first and second components 901 and 911. Similar to the other cutting devices described herein, the cutting device 900 includes teeth on each component 901, 911, respectively, that can shear against each other during rotation of one or both of the components 901, 911, to remove small pieces of tissue. Unlike the embodiment of FIG. 7, the first component 901 of the embodiment of FIG. 13 has two cutting elements 912 and two intake windows 914 for drawing in and removing tissue.

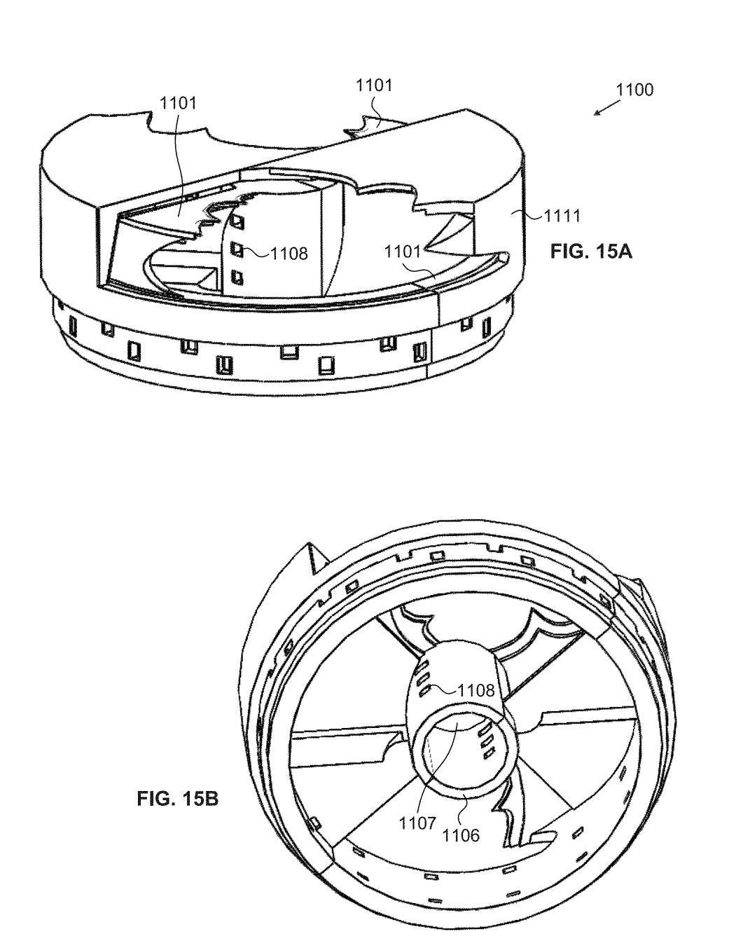

FIG. 16 provides a perspective view of a working end of a cutting device 1200 having first and second components 1201 and 1211. The device includes inner and outer crown cutters having teeth 1205 and 1215 extending substantially axially. The teeth 1205 are configured to bore into a material without any additional cutting elements. This embodiment omits the inner cutting element of the first component shown in FIG. 7.

Numerous variations on these embodiments are possible and include those, mutatis mutandis, set forth regard to any of the other embodiment set forth herein.

FIGS. 10A-10B provide a perspective and a perspective cut view respectively of a working end of a cutting device 600. The cutting device 600 includes first and second components 601 and 611 attached to a central shaft 640. The first component 601 includes an inner cutting element 602, having two primary cutting surfaces 603 and two secondary cutting surfaces 604. The second component 611 includes two outer cutting elements 612, having a primary surface 613 and a secondary surface 614. When assembled, first component 601 is disposed radially inward of second component 611. The first component 601 is capable of rotating relative to the second component 611 to cause shearing away of tissue or other material as the inner and outer cutting elements rotate past one another. In some embodiments, the first component can also be configured to rotate about the central longitudinal axis 621. For example, the first component can rotate counter-clockwise, and the second component can rotate clockwise.

Both the inner and outer cutters 602, 612 have two-sided radial extending, and slightly azimuthal extending, primary cutting surfaces 603 and 613, respectively. The primary cutting surfaces 603 and 613 can include teeth 607 and 617. Moreover, both the inner and outer cutters 602, 612 have secondary cutting surfaces 604 and 614 extending axially at the radial extremes of the components, which can also include teeth 605 and 615. An intake window 622 of the device consists of two opposite facing 90 degree wedges for drawing in tissue to be sheared between the rotating cutting surfaces and two sloping surfaces for drawing the sheared tissue proximally.

Advantageously, the distal end of the cutter 600 can have a flat portion 630 that extends from the outer circumference to the radial center of the cutter 600. The flat portion 630 can have an axial thickness of less than 100 microns, such as less than 50 microns. The spatial relationships between the flat surface, the cutting elements 603 and 613 and the intake windows 622 can allow for removal of tissue along a single plane, such as during a milling process, thereby avoiding removal of unwanted tissue.

Further, the cutter 600 can be a micro-scale device. Thus, the diameter of the device 600 can be less than less than 5 mm, such as less than 3 mm, e.g. less than 1 mm. The minimum feature size (e.g., the size of teeth 605 and 615) can be on the order of tens of microns, i.e. less than 100 microns, such as less than 50 microns. Moreover, the precision of the device build can be on the micron level, i.e. between 1 and 10 microns. Having a micro-scale milling device can advantageously allow the device to be used in small areas of the body that are unreachable by larger devices. Moreover, the precision of the build and the minimum feature size can be useful for very precise and specific tissue or material cutting. For example, tissue having a diameter of less than 5 microns, such as between 2 and 5 microns, can be removed during a milling process. Removing such small pieces avoids excessive damage to surrounding tissue. These micro-scale devices may be made using the electrochemical fabrication process described above.

Numerous variations of the cutter 600 exist, some of which are similar, mutatis mutandis, to those noted above. Additional variations may include the removal of the central rod shaft or the hollowing out of the shaft to form a ring element through which a guide wire, imagining device or other component may extend. In still other embodiment variations, the central rod may be a hollow shaft with perforation and may be connected to a proximal tube (e.g. with a rotatable coupling) that allows a flow of an irrigation fluid to be directed into the working region e.g. for aspiration along with removed material.

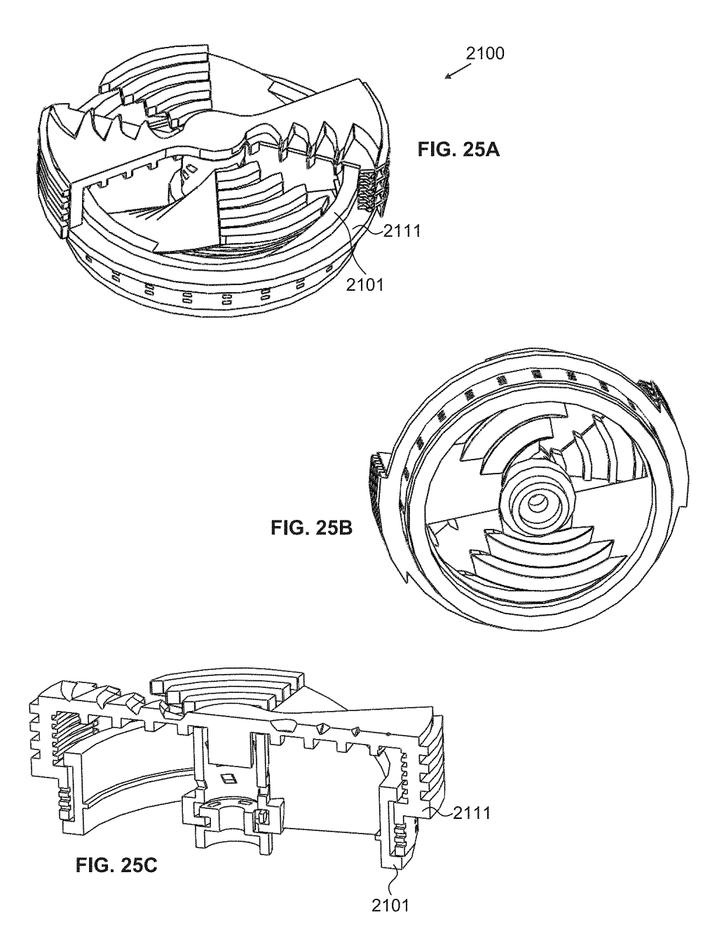

FIGS. 11, 12, 14, 15, 17, 23, 25 show similar devices to device 600, i.e., that include two rotating portions having flat distal surfaces. Thus, the devices can include many of the same features and advantages as device 600 and may be made using the electrochemical fabrication process described above.

FIGS. 11A and 11B provide a perspective and a perspective cut view respectively of a working end of a cutting device 700 having first and second components 701 and 711. The cutting device 700 is similar to that of cutter 600 with the exception of a different set of primary cutting blade configurations 703 and 713.

FIG. 12 provides a perspective view of a working end of a cutting device 800 having first and second components 801 and 811 that are configured to be rotated with respect to each other, as in the embodiments described above. The cutting device 800 has an inner cutter similar to that of cutter 600 with the exception that the central rod or shaft is removed so that a guide wire, imaging device or other element may be extended down the central axis of the device. The device also lacks an outer cutting element. Numerous variations of cutter 800 exist some of which are similar, mutatis mutandis, to those noted above with regard to the other embodiments set forth herein above and herein after. An additional variation of the device might include the complete removal of the outer component 811 and any tube used to hold or control its motion and instead simply allow the device to extend from and rotate within a catheter or other delivery lumen.

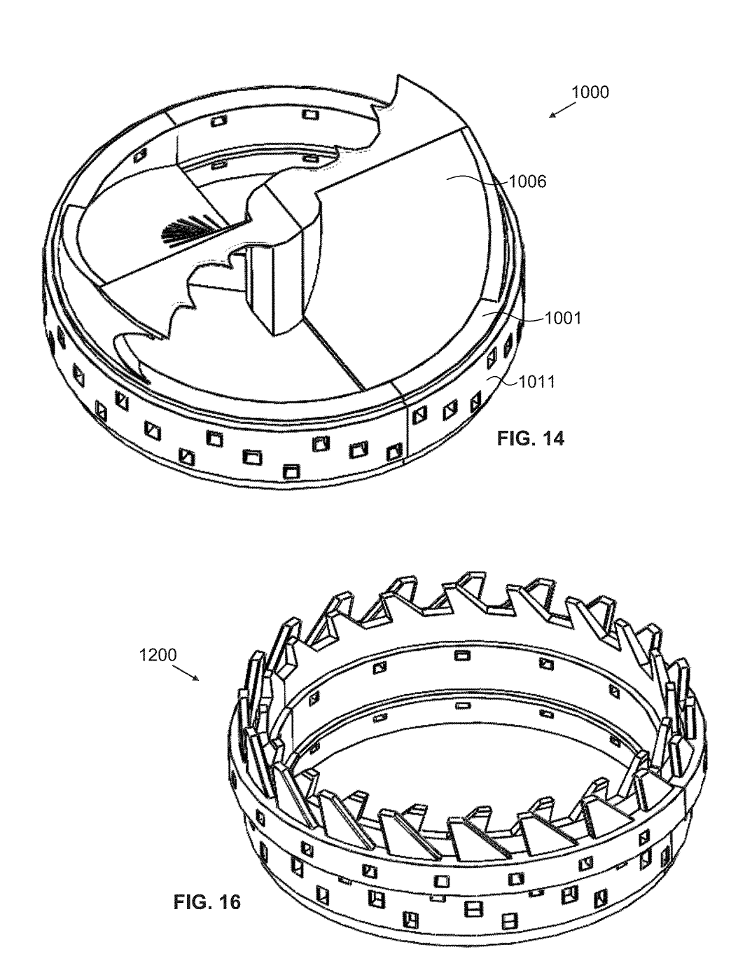

FIG. 14 provides a perspective view of a working end of a cutting device 1000 having first and second components 1001 and 1011. The device has an inner cutter 1006 similar to that of cutter 600 of the invention but lacks an outer cutter. Numerous variations of cutter 1000 exist some of which are similar, mutatis mutandis, to those noted above with regard to the other embodiments set forth herein above and herein after. An additional variation of the device might include the complete removal of the outer component 1011 and any tube used to hold or control its motion and instead simply allow the device to extend from and rotate within a catheter or other delivery lumen.

FIG. 15A-15B provide a perspective views of a working end of a cutting device 1100 having first and second components 1101 and 1111. The device is similar to that of cutter 600 except that the central shaft 1106 includes a hollow center 1107 with irrigation apertures 1108. A rotating or non-rotating tube may be connected to this central shaft to provide a flow of irrigation fluid. Numerous variations of cutter 1100 exist some of which are similar, mutatis mutandis, to those noted above with regard to the other embodiments set forth herein above and herein after. Additional variations of the device might include variations on the number, position and orientation of the apertures so that a desired flow volume and flow direction can be obtained.

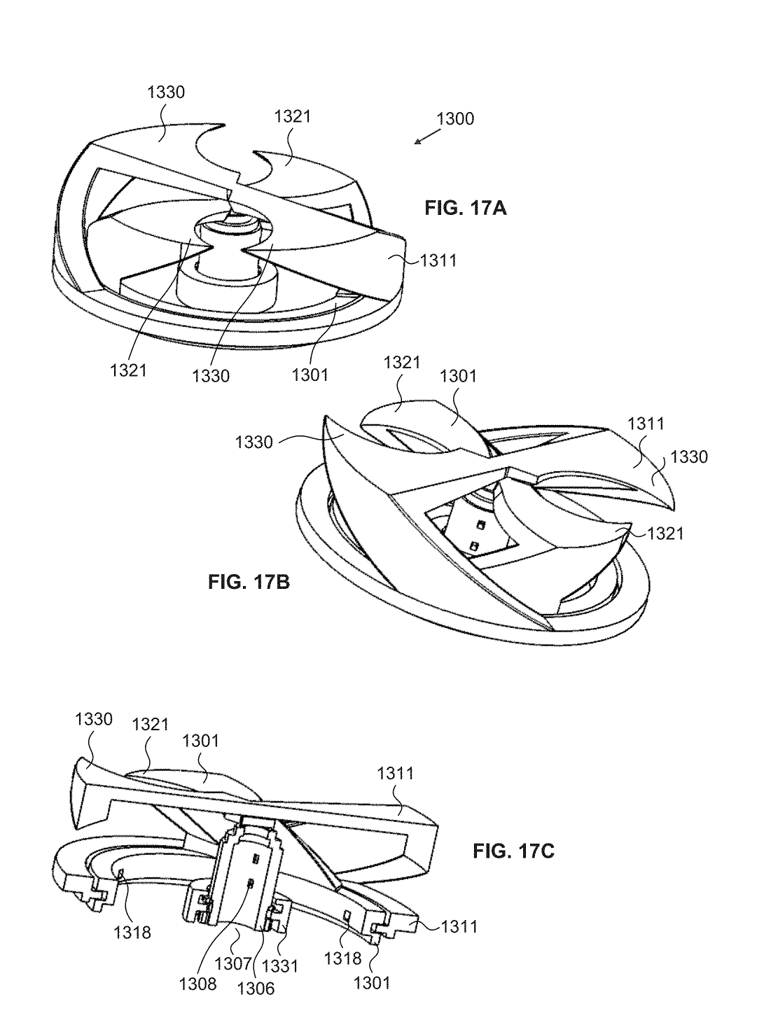

FIGS. 17A-17C provide various perspective views of a working end of a cutting device 1300 having first and second components 1301, 1311. Component 1301 includes a pair of inner pinch-off cutters 1321, and component 1311 includes a pair of outer pinch-off cutters 1330. In addition, the cutting device includes a third inner component 1331. The device 1300 further includes a central irrigation tube 1306 including passage 1307 and apertures 1308 that forms part of component 1301. Relative rotation between cutters 1321 and 1331 shears tissue extending into the openings between the cutters. Component 1331 provides a tube coupler that is capable of relative rotation relative to the irrigation tube 1306 so that the feed tube can provide fluid for irrigation but need not rotate in unison with the inner cutter. The inside portion of the outer ring of the component 1301 also include inward facing aperture 1318, which may exist solely for fabrication purposes (e.g. release of sacrificial material) or may provide for additional irrigation fluid which may be supplied between a tube connecting to component 1301 and a tube connecting to component 1311. Numerous variations on this embodiment are possible and include those, mutatis mutandis, set forth regard to the various other embodiments set forth herein. Other variations might include a coupling between the irrigation tube and the inner cutting element so that these components can rotate relative to one another.

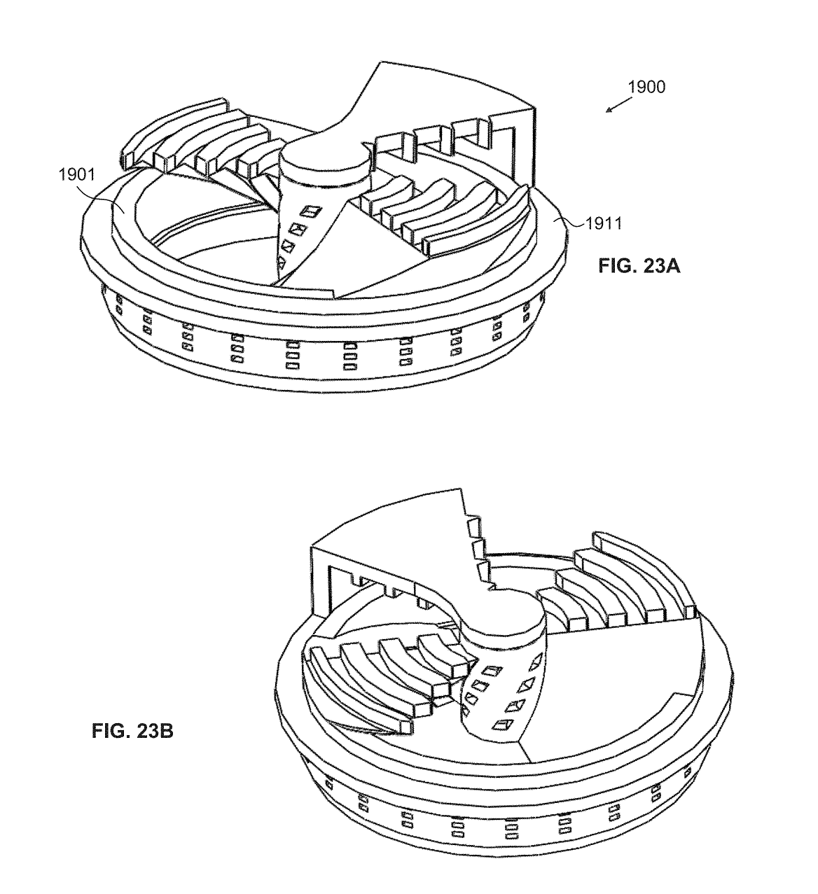

FIG. 23 illustrates several embodiments of additional cutting devices having rotating parts and a flat distal end. The embodiments shown therein have features that include various combinations or refinement of the features included in the other embodiments presented herein.

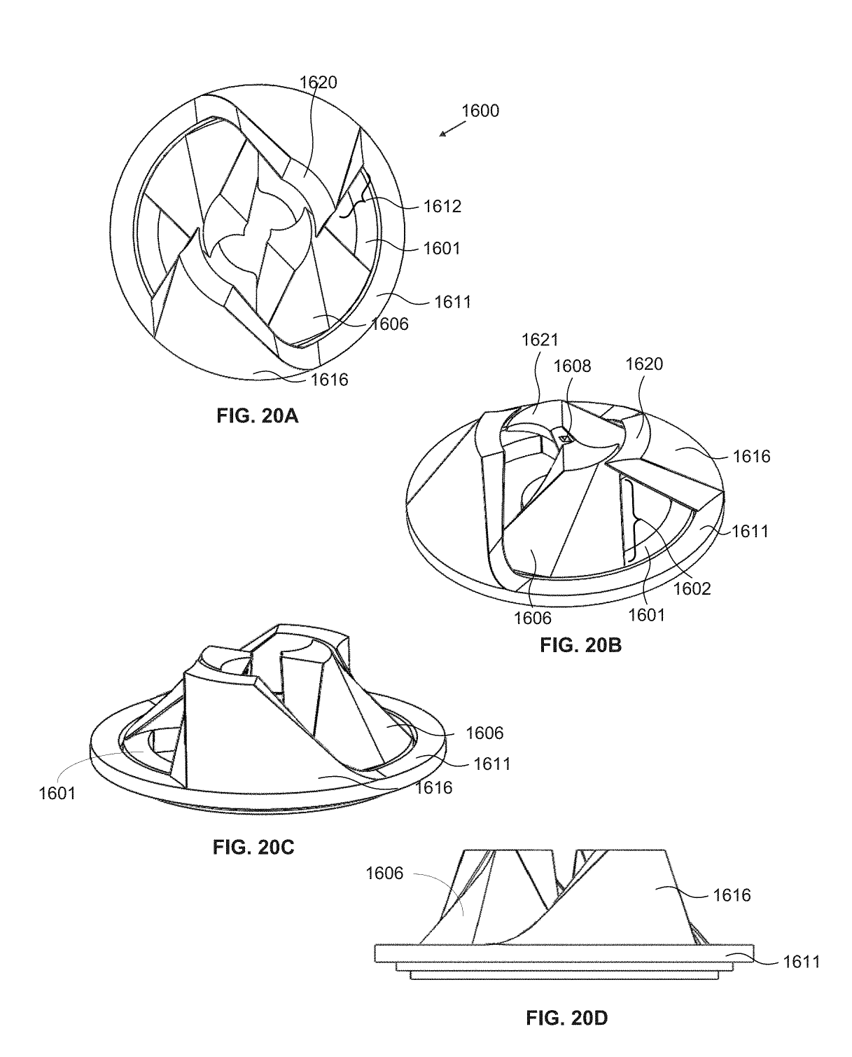

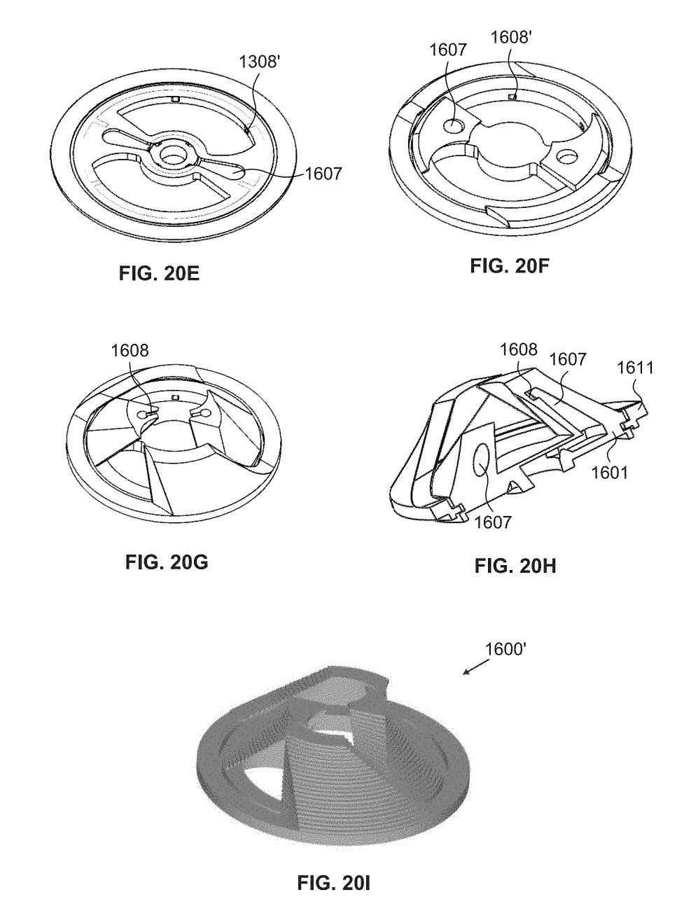

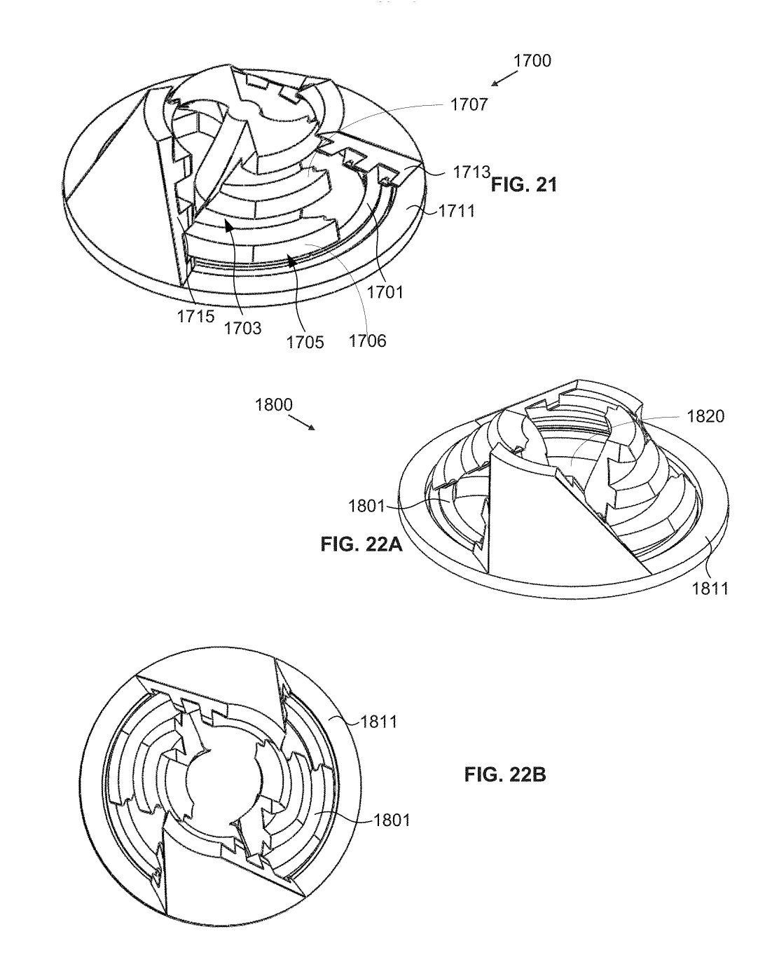

FIGS. 20A-20H provide perspective views of a working end of a cutting device 1600. The cutting device 1600 includes first and second components 1601 and 1611 which can be rotated with respect to each other. The components 1600, 1611 each include a conical cutting element 1606, 1616 extending axially. The conical cutting elements can together form a helical shape. The helical shape can be advantageous for particular medical processes, such as drilling, The edges 1620 of conical element 1616 and edges 1621 of conical element 1606 can be sharp such that the shearing action from rotation of the edges relative to one another causes the cutter 1600 to drill through material, such as tissue. Further, the edges 1620 and 1621 can have a beveled shape. The beveled edges 1620 can advantageously promote shearing. The beveled edge can have a thickness that is less than 10 microns, such as between 2 and 5 microns, allowing for precise tissue cutting.

The device 1600 also includes pairs of inner and outer cutters elements 1602 and 1612, respectively, extending axially, radially inward from ring-like base structures of components 1601 and 1611, and extending forward azimuthally, as part of components 1601 and 1611 respectively. Component 1601 also includes irrigation channels 1607 leading to irrigation apertures 1608 and 1608′ on the cutting blade and on the ring-like base structure. The cutting device 1600 can be a micro-scale device such that it can be used in small areas of the body that are unreachable by larger devices, such as blood vessels having a diameter of less than 5 mm, such as less than 5 mm, such as less than 3 mm, e.g. less than 1 mm. Moreover, the precision of the build and the minimum feature size can be useful for very precise and specific tissue or material cutting. For example, tissue having a diameter of less than 5 microns, such as between 2 and 5 microns, can be removed. Removing such small samples avoids excessive damage to surrounding tissue.

FIG. 20I provides an example layered device 1600′ as the devices of FIGS. 20A-20H might be formed from a plurality of adhered layers which might be produced in a multi-layer, multi-material fabrication process (e.g. the electrochemical fabrication process described above).

Numerous variations on this embodiment are possible and include those, mutatis mutandis, set forth regard to the various other embodiments set forth herein. Other variations might include inner and/or outer blade configuration that provide for tight fitting blades while minimizing risk of tolerance based collisions by offsetting regions of initial passing (e.g. tips) radially inward (in the case of the inner cutting blades) or outward (in the case of the outer blades) in to ensure smooth passing while providing tightened ring-like base clearances or clearances on portions of the blades that are recessed from the initial contact regions. Variations of the device of this embodiment, like other embodiments described herein, can also provide for an open central region so that a guide wire, imaging device, or other tool or instrument may be moved down the center of the cutting element. The open central region may be defined by the blades themselves or by a ring like structure, with or without, a coupling element through which the central instrument may pass.

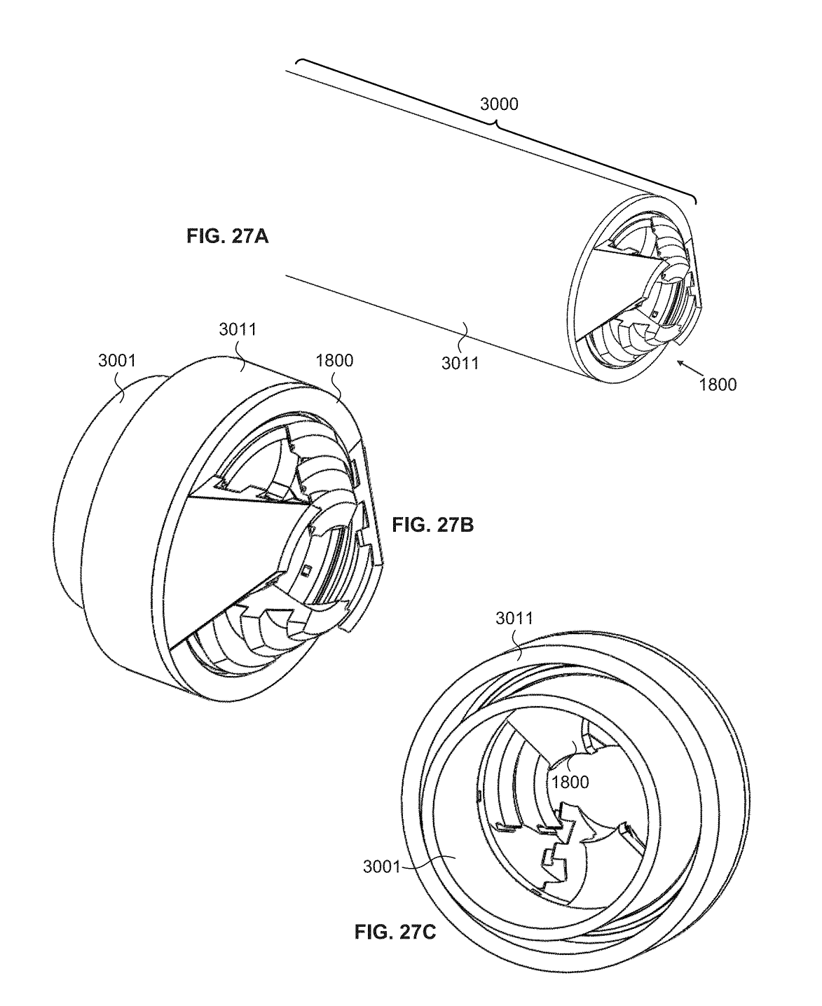

FIGS. 22 and 27 show similar devices to device 600, i.e., that include a conical-shaped distal end. Thus, the devices can include many of the same features and advantages as device 600 and may be made using the electrochemical fabrication process described above.

FIGS. 22A-22B provide perspective views of a working end of a cutting device 1800 having first and second components 1801 and 181. Variations of the device 1800 are similar, mutatis mutandis, to those for the other embodiments, noted herein and as with the other embodiments may include features or portions of features found only within the other embodiments themselves. Variations of the device may include a ring-like structure or structures which guide movement for an instrument inserted through the center 1820 of the cutting device so that the instrument cannot inadvertently get caught by the cutting blades themselves.

FIGS. 27A-27C provide various views of an example device 3000 including a working end of an example cutting element 1800 having its inner and outer cutting elements coupled to inner and outer tubes 3001 and 3011 respectively which can be used to rotate the cutting elements or to hold them stationary. FIGS. 27B and 27C provide truncated views of the tubes so that the inner tube may be seen. Variations of this embodiment may make use of the working ends of the other embodiments set forth herein or variations thereof. In other alternatives, the tubes or the working ends themselves may include pivot elements or bendable elements to provide a desired orientation to cutting elements when in use. Further alternatives may include the use of additional tubes or fewer tubes as appropriate. In use, various fluids or vacuum may be applied between the tubes to provide desired lubrication, irrigation, aspiration, drug delivery, or the like.

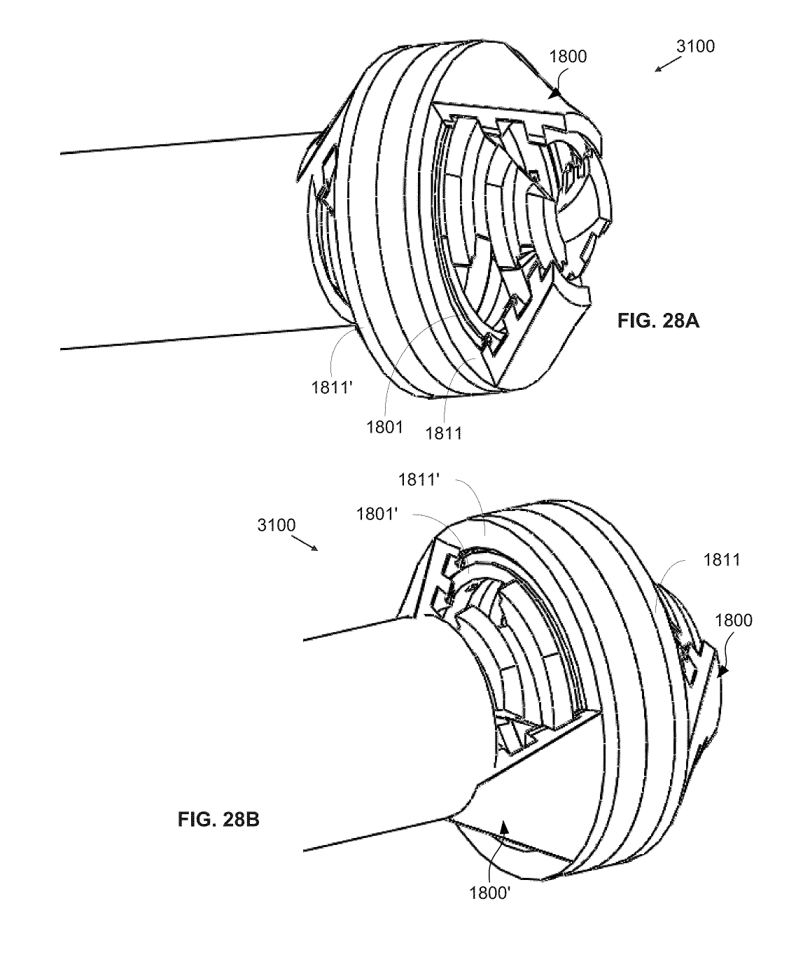

In some configurations, the cutting devices described herein can be stacked or combined to further cut tissue brought into the tube. Referring to FIGS. 28A-28B, the first device 1800 can include an inner component 1801 and an outer component 1811, which can be designed similar to any of the first and second components described herein. A second device 1800′ can be combined with the first device 1800, such as stacked together axially as shown in FIGS. 28A-28B. The second device 1800′ can include an inner component 1801′ and an outer component 1811′, which can be designed similar to any of the first and second components described herein. Optionally, as shown in FIGS. 28A-28B, the first device 1800 can be a forward-facing cutter, while the second device 1800′ can be a backward-facing cutter relative to the control tubes.

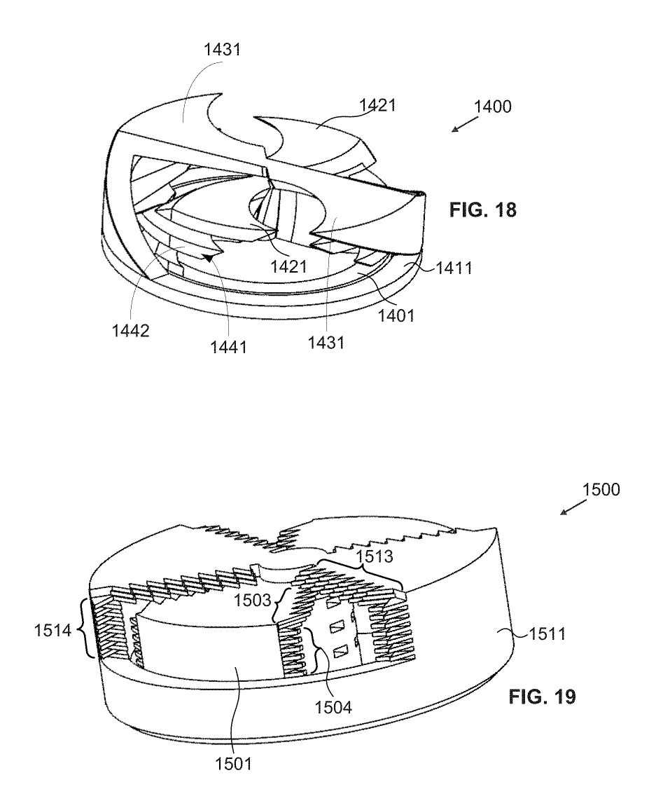

Referring to FIGS. 18, 19, 21, 24, and 26, the cutting devices described herein can be configured to include multiple cutters along the axial and/or radial directions. Having multiple cutters along the axial and/or radial directions can advantageously allow for better shearing of tissue.

FIG. 18 provides a perspective view of a working end of a cutting device 1400 having first and second components 1401, and 1411. Component 1401 includes a pair of inner pinch-off cutters 1421 spaced apart circumferentially, and component 1411 includes a pair of outer pinch-off cutters 1431 spaced apart circumferentially. The inner and outer cutting blades also include interlaced side cutters 1441 that provide for side milling. The side cutters 1441 can each include cutting surfaces 1442 that are parallel to the central axis of the cutter 600. The cutting surfaces 1442 can extend along the same radial plane. The side cutters 1441 can be spaced apart axially. Moreover, each pinch-off cutter 1421 can include a set of side cutters 1441 approximately axially aligned thereto. Further, the side cutters 1441 can each include parallel cutting surfaces 1443 extending perpendicular to the central axis of the cutter 600. The outer component 1411 can include similar cutting surfaces such that the side cutters of the inner and outer components 1401 and 1411 can interlace with one another. The axial thickness of each side cutter 1441 can be less than 100 microns, such as less than 50 microns. The interaction of the side cutters and/or the pinch-off cutters as one or both of the components 1401, 1411 rotates can allow for shearing of tissue. Numerous variations on this embodiment are possible and include those, mutatis mutandis, set forth regard to the various other embodiments set forth herein. Other variations might include different numbers of interlaced elements, different thicknesses of interlaced elements, and different interlacing depths for those elements.

As shown in FIG. 19, a cutting device 1500 can include first and second components 1501 and 1511. The device 1500 includes stacked levels of cutters 1504 on primary cutting elements of both the inner and outer components. The device 1500 further includes side teeth 1514 that provide for retention and shredding of material. The teeth of the inner and outer elements can both extend perpendicular to the central axis of the device and can be located on opposing planes so as to allow shearing when the components 1501, 1511 are rotated relative to one another. The teeth can have an axial thickness of less than 100 microns, such as less than 50 microns, such as less than 10 microns. The device 1500 can also include irrigation apertures on central shaft. Other variations might include different numbers and configurations of stacked cutter primary and secondary cutting teeth.

FIG. 21 provides a perspective view of a working end of a cutting device 1700 having first and second components 1701, and 1711. The first component 1701 includes inner cutting blades 1703 spaced apart circumferentially, while the second component 1711 includes outer cutting blades 1713 spaced apart circumferentially. The inner cutting blades 1703 are provided with outward facing side teeth 1705 that interlace with inward facing side teeth 1715 on the outer cutting blades 1711 to shear tissue as the first and second components rotate with respect to each other. The teeth 1705 can be stacked and spaced apart axially. The teeth 1705 can each include a surface 1706 parallel to the central axis of the device and a surface 1707 perpendicular to the central axis of the device. The surfaces 1706 extending approximately parallel with each other can each be located along a different radial dimension so as to create a conical-shaped distal end of the device. Variations of the device 1700 are similar, mutatis mutandis, to those for the other embodiments, noted herein and as with the other embodiments may include features or portions of features found only within the other embodiments themselves. Variations of the device may include irrigation channels and apertures.

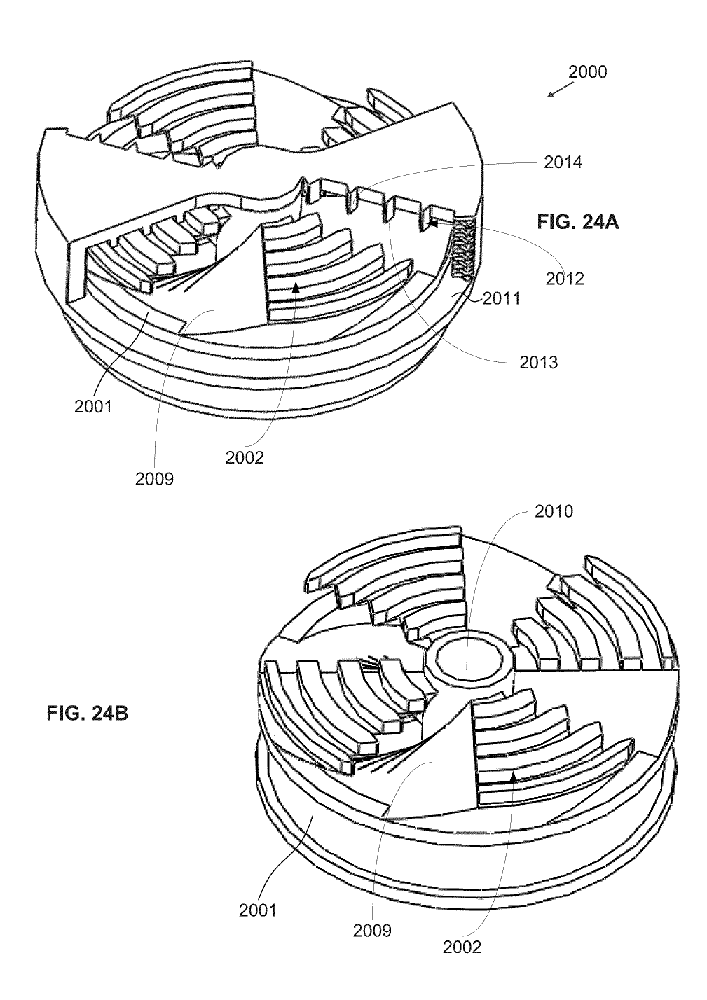

As shown in FIGS. 24A-24B, a cutting device 2000 can include an outer component 2011 and an inner component 2001. The outer component 2011 can include axially-extending cutting elements 2012. The axially-extending cutting elements 2012 can each have a cutting surface 2013 extending parallel to the central axis of the device and a cutting surface 2014 extending perpendicular to the central axis of the device. The axially-extending cutting elements can be spaced apart radially and/or circumferentially. Likewise, the inner component 2001 can include similar axially-extending elements 2002. The axially extending elements 2002 can be spaced apart radially and/or circumferentially. Further, the inner cutting element 2001 can include one or more sloped surfaces 2009 such that the inner cutting elements 2001 can be spaced apart axially. The axially-extending elements of each component can extend along a common axial plane. The interaction of the surfaces of the cutting elements 2012 and 2002 as one or both of the elements 2001, 2011 rotates, can allow for shearing of tissue. In the illustrated embodiment, outer component 2011 has a shaft (not shown) that fits into a bore 2010 formed in inner component 2001.

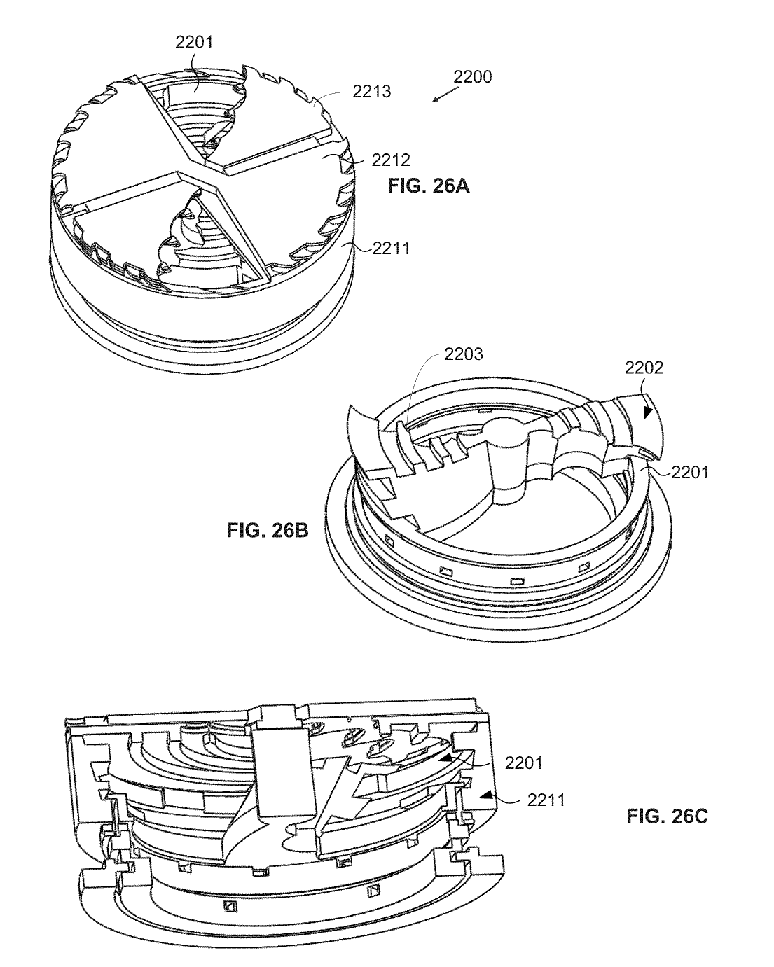

As shown in FIGS. 26A-26C, a cutting device 2200 can include an inner component 2201 and an outer component 2211. The inner component 2201 can include cutting surfaces 2202 having teeth 2203, while the outer component 2211 can include cutting surfaces 2212 having 2213. Inner component 2201 and outer component 2211 may be rotated with respect to each other so that cutting surfaces 2202 and 2212 can shear tissue extending through intake windows 2216. Other embodiments are possible. For example, the inner cutting element can include multiple cutters extending radially, while the outer cutting element includes multiple cutters extending axially. Alternatively, the inner cutting element can include multiple cutters extending axially while the outer cutter element also includes multiple cutters extending axially.

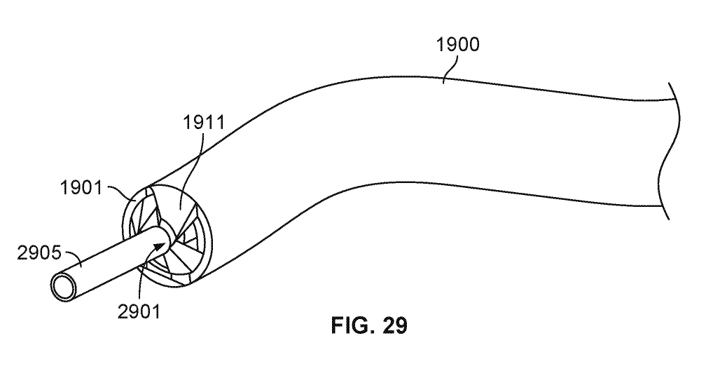

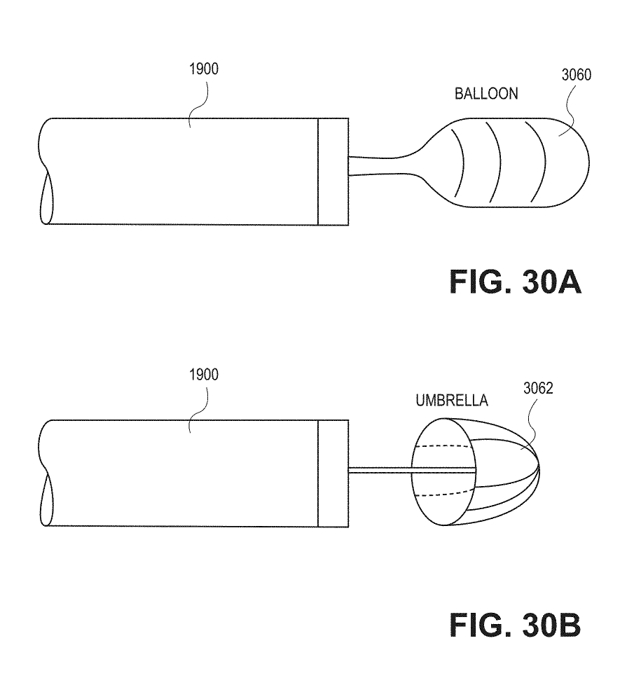

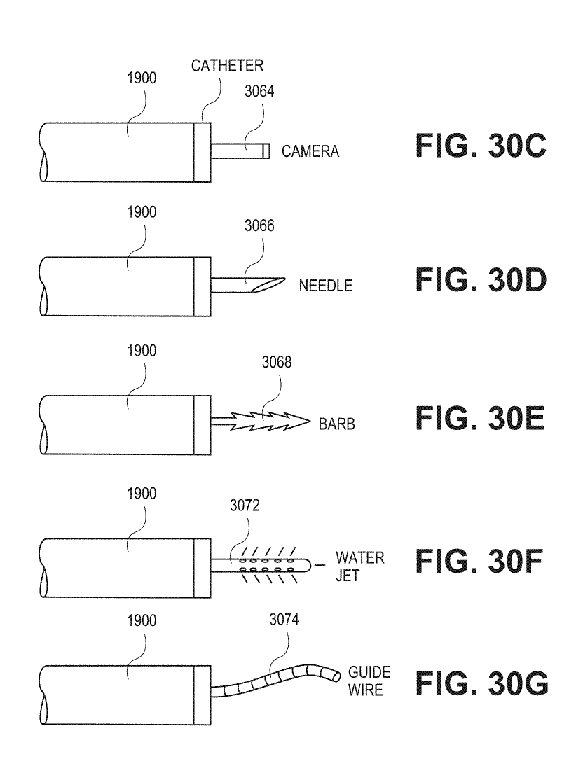

Further, referring to FIG. 29, the devices described herein, due to their small features sizes and precise build, can advantageously be configured to include ancillary components that extend along the inner central axis and through an opening in the distal end of the device. The cutting device 1900, representing any of the cutting devices described herein, can include including inner and outer cutting elements 1901 and 1911. A hole 2901 can extend along the central axis of the cutting device 1900. As such, an ancillary component 2905 can extend through the cutting device 1900. Referring to FIG. 30A, the ancillary component can be a balloon 3060. Referring to FIG. 30B, the ancillary component can be an umbrella 3062. Referring to FIG. 30C, the ancillary component can be an imaging element 3064, such as a CMOS camera, a fiber optic scope with CCD or CMOS, 2D and 3D capture and display, ultrasound (IVUS), Doppler, or birefringence-insensitive optical coherence tomography (OCT). Referring to FIG. 30D, the ancillary component can be a needle 3066, such as drug delivery needle. Referring to FIG. 30E, the ancillary component can be a longitudinal element 3068 including barbs to, for example, gather tissue and pull it towards the cutting elements or to stabilize tissue during cutting. Referring to FIG. 30F, the ancillary component can be a water jet tube 3072, such as a water jet tube for delivering water to clear clots. Referring to FIG. 30G, the ancillary component can be a guide wire 3074. Additional ancillary components include a device for suction, a device for irrigation, or an energy system to coagulate or cauterize, such as a system providing RF energy, an argon beam, a laser, or a DC current.

In summary, various specific cylindrical cutting device embodiments have been taught herein. These various device embodiments may make use of various elements including: (1) designs are driven with 2 concentric tubes; (2) cutting surfaces that face forward with respect to the longitudinal axis of the tool or instrument; (3) an inside tube is connected to one set of blades; (4) an outside tube is connected to one set of blades; (5) an inside tube is rotated with respect to the outside tubes, making the cutting blades pass one another; (6) in some cases the outside tube can be rotated in either direction at a different rate than the inside tube to expose all blades to the tissue at all azimuthal angles (this allows cutting over the entire front surface of the targeted area); (7) the various device embodiments can be attached to articulating tubes so that the cutting end can be steerable; (8) the various device embodiments can incorporate aspiration to remove the material that has been cut; (9) some embodiments may provide turbine or propeller-like effects which will help material transport away from the targeted area; (10) some embodiments may incorporate irrigation to aid in the material transport; (11) some embodiments may incorporate central imaging; (12) some embodiments may be deliverable via a central guide wire; (13) various embodiments are scalable to different radial sizes from less than one-half millimeter to more than a centimeter; and/or (14) some embodiments may be assisted by one or more proximally located supplement cutters, shredders, or mechanical flow assist devices.

### FURTHER COMMENTS AND CONCLUSIONS

Structural or sacrificial dielectric materials may be incorporated into embodiments of the present invention in a variety of different ways. Such materials may form a third material or higher deposited on selected layers or may form one of the first two materials deposited on some layers. Additional teachings concerning the formation of structures on dielectric substrates and/or the formation of structures that incorporate dielectric materials into the formation process and possibility into the final structures as formed are set forth in a number of patent applications filed Dec. 31, 2003. The first of these filings is U.S. Patent Application No. 60/534,184 which is entitled “Electrochemical Fabrication Methods Incorporating Dielectric Materials and/or Using Dielectric Substrates”. The second of these filings is U.S. Patent Application No. 60/533,932, which is entitled “Electrochemical Fabrication Methods Using Dielectric Substrates”. The third of these filings is U.S. Patent Application No. 60/534,157, which is entitled “Electrochemical Fabrication Methods Incorporating Dielectric Materials”. The fourth of these filings is U.S. Patent Application No. 60/533,891, which is entitled “Methods for Electrochemically Fabricating Structures Incorporating Dielectric Sheets and/or Seed layers That Are Partially Removed Via Planarization”. A fifth such filing is U.S. Patent Application No. 60/533,895, which is entitled “Electrochemical Fabrication Method for Producing Multi-layer Three-Dimensional Structures on a Porous Dielectric”. Additional patent filings that provide teachings concerning incorporation of dielectrics into the EFAB process include U.S. patent application Ser. No. 11/139,262, filed May 26, 2005 by Lockard, et al., and which is entitled “Methods for Electrochemically Fabricating Structures Using Adhered Masks, Incorporating Dielectric Sheets, and/or Seed Layers that are Partially Removed Via Planarization”; and U.S. patent application Ser. No. 11/029,216, filed Jan. 3, 2005 by Cohen, et al., now abandoned, and which is entitled “Electrochemical Fabrication Methods Incorporating Dielectric Materials and/or Using Dielectric Substrates”. These patent filings are each hereby incorporated herein by reference as if set forth in full herein.

Some embodiments may employ diffusion bonding or the like to enhance adhesion between successive layers of material. Various teachings concerning the use of diffusion bonding in electrochemical fabrication processes are set forth in U.S. patent application Ser. No. 10/841,384 which was filed May 7, 2004 by Cohen et al., now abandoned, which is entitled “Method of Electrochemically Fabricating Multilayer Structures Having Improved Interlayer Adhesion” and which is hereby incorporated herein by reference as if set forth in full. This application is hereby incorporated herein by reference as if set forth in full.

Some embodiments may incorporate elements taught in conjunction with other medical devices as set forth in various U.S. patent applications filed by the owner of the present application and/or may benefit from combined use with these other medical devices: Some of these alternative devices have been described in the following previously filed patent applications: (1) U.S. patent application Ser. No. 11/478,934, by Cohen et al., and entitled “Electrochemical Fabrication Processes Incorporating Non-Platable Materials and/or Metals that are Difficult to Plate On”; (2) U.S. patent application Ser. No. 11/582,049, by Cohen, and entitled “Discrete or Continuous Tissue Capture Device and Method for Making”; (3) U.S. patent application Ser. No. 11/625,807, by Cohen, and entitled “Microdevices for Tissue Approximation and Retention, Methods for Using, and Methods for Making”; (4) U.S. patent application Ser. No. 11/696,722, by Cohen, and entitled “Biopsy Devices, Methods for Using, and Methods for Making”; (5) U.S. patent application Ser. No. 11/734,273, by Cohen, and entitled “Thrombectomy Devices and Methods for Making”; (6) U.S. Patent Application No. 60/942,200, by Cohen, and entitled “Micro-Umbrella Devices for Use in Medical Applications and Methods for Making Such Devices”; and (7) U.S. patent application Ser. No. 11/444,999, by Cohen, and entitled “Microtools and Methods for Fabricating Such Tools”. Each of these applications is incorporated herein by reference as if set forth in full herein.

Though the embodiments explicitly set forth herein have considered multi-material layers to be formed one after another. In some embodiments, it is possible to form structures on a layer-by-layer basis but to deviate from a strict planar layer on planar layer build up process in favor of a process that interlaces material between the layers. Such alternative build processes are disclosed in U.S. application Ser. No. 10/434,519, filed on May 7, 2003, now U.S. Pat. No. 7,252,861, entitled Methods of and Apparatus for Electrochemically Fabricating Structures Via Interlaced Layers or Via Selective Etching and Filling of Voids. The techniques disclosed in this referenced application may be combined with the techniques and alternatives set forth explicitly herein to derive additional alternative embodiments. In particular, the structural features are still defined on a planar-layer-by-planar-layer basis but material associated with some layers are formed along with material for other layers such that interlacing of deposited material occurs. Such interlacing may lead to reduced structural distortion during formation or improved interlayer adhesion. This patent application is herein incorporated by reference as if set forth in full.

The patent applications and patents set forth below are hereby incorporated by reference herein as if set forth in full. The teachings in these incorporated applications can be combined with the teachings of the instant application in many ways: For example, enhanced methods of producing structures may be derived from some combinations of teachings, enhanced structures may be obtainable, enhanced apparatus may be derived, and the like.

| US Pat App No., Filing Date | |
|---|---|
| US App Pub No., Pub Date | |
| US Patent No., Pub Date | Inventor, Title |
| 09/493,496 - Jan. 28, 2000 | Cohen, “Method For Electrochemical Fabrication” |
| -- | |
| PAT 6,790,377 - Sep. 14, 2004 | |
| 10/387,958 - Mar. 13, 2003 | Cohen, “Electrochemical Fabrication Method and |
| 2003-022168A - Dec. 4, 2003 | Application for Producing Three-Dimensional |
| -- | Structures Having Improved Surface Finish” |
| 10/434,294 - May 7, 2003 | Zhang, “Electrochemical Fabrication Methods With |
| 2004-0065550A - Apr. 8, 2004 | Enhanced Post Deposition Processing” |
| -- | |
| 10/434,295 - May 7, 2003 | Cohen, “Method of and Apparatus for Forming Three- |
| 2004-0004001A - Jan. 8, 2004 | Dimensional Structures Integral With Semiconductor |
| -- | Based Circuitry” |
| 10/434,103 - May 7, 2004 | Cohen, “Electrochemically Fabricated Hermetically |
| 2004-0020782A - Feb. 5, 2004 | Sealed Microstructures and Methods of and Apparatus |
| PAT 7,160,429 - Jan. 9, 2007 | for Producing Such Structures” |
| 10/841,006 - May 7, 2004 | Thompson, “Electrochemically Fabricated Structures |
| 2005-0067292 - May 31, 2005 | Having Dielectric or Active Bases and Methods of and |
| -- | Apparatus for Producing Such Structures” |
| 10/434,519 - May 7, 2003 | Smalley, “Methods of and Apparatus for |
| 2004-0007470A - Jan. 15, 2004 | Electrochemically Fabricating Structures Via Interlaced |
| PAT 7,252,861 - Aug. 7, 2007 | Layers or Via Selective Etching and Filling of Voids” |
| 10/841,347 - May 7, 2004 | Cohen, “Multi-step Release Method for |
| 2005-0072681 - Apr. 7, 2005 | Electrochemically Fabricated Structures” |
| -- | |
| 60/534,183 - Dec. 31, 2003 | Cohen, “Method and Apparatus for Maintaining |
| -- | Parallelism of Layers and/or Achieving Desired |
| -- | Thicknesses of Layers During the Electrochemical |
| | Fabrication of Structures” |
| 11/733,195 - Apr. 9, 2007 | Kumar, “Methods of Forming Three-Dimensional |
| 2008-0050524 - Feb. 28, 2008 | Structures Having Reduced Stress and/or Curvature” |
| -- | |
| 11/506,586 - Aug. 8, 2006 | Cohen, “Mesoscale and Microscale Device Fabrication |
| 2007-0039828 - Feb. 22, 2007 | Methods Using Split Structures and Alignment |
| -- | Elements” |
| 10/949,744 - Sep. 24, 2004 | Lockard, “Three-Dimensional Structures Having |
| 2005-0126916 - Jun. 16, 2005 | Feature Sizes Smaller Than a Minimum Feature Size |
| PAT 7,498,714 - Mar. 3, 2009 | and Methods for Fabricating” |
| 14/634,424 - Feb. 27, 2015 | Lockard, “Miniature Shredding Tool for Use in Medical |
| 2015-0173788 - Jun. 25, 2015 | Applications and Methods for Making” |
| -- | |
| 14/452,376 - Aug. 5, 2014 | Schmitz, “Selective Tissue Removal Tool for Use in Medical |
| 2014-0350567 - Nov. 27, 2014 | Applications and Methods for Making and Using” |
| -- | |
| 15/277,916 - Sep. 27, 2016 | Schmitz, “MEMS Micro Debrider Devices and Methods of |
| 2017-0014148 - Jan. 19, 2017 | Tissue Removal” |
| -- | |
| 15/005,994 - Jan. 25, 2016 | Schmitz, “Minimally Invasive Micro Tissue Debriders Having |
| 2016-0135831 - May 19, 2016 | Targeted Rotor Positions |
| -- | |
| 13/843,462 - Mar. 15, 2013 | Schmitz, “MEMS Debrider Drive Train” |
| 2014-0148836 - May 29, 2014 | |
| -- | |
| 15/167,899 - May 27, 2016 | Schmitz, “Solderless Microcircuit Boards, Components, |
| -- | Methods of Making, and Methods of Using” |
| -- | |
| 15/292,029 - Oct. 12, 2016 | Schmitz, Surgical Micro-Shears and Methods of Fabrication |
| 2017-0095264 - Sep. 6, 2017 | and Use |
| -- | |

Though various portions of this specification have been provided with headers, it is not intended that the headers be used to limit the application of teachings found in one portion of the specification from applying to other portions of the specification. For example, it should be understood that alternatives acknowledged in association with one embodiment, are intended to apply to all embodiments to the extent that the features of the different embodiments make such application functional and do not otherwise contradict or remove all benefits of the adopted embodiment. Various other embodiments of the present invention exist. Some of these embodiments may be based on a combination of the teachings herein with various teachings incorporated herein by reference.

It is intended that the aspects of the invention set forth herein represent independent invention descriptions which Applicant contemplates as full and complete invention descriptions that Applicant believes may be set forth as independent claims without need of importing additional limitations or elements, from other embodiments or aspects set forth herein, for interpretation or clarification other than when explicitly set forth in such independent claims once written. It is also understood that any variations of the aspects set forth herein represent individual and separate features that may form separate independent claims, be individually added to independent claims, or added as dependent claims to further define an invention being claimed by those respective dependent claims should they be written.

In view of the teachings herein, many further embodiments, alternatives in design and uses of the embodiments of the instant invention will be apparent to those of skill in the art. As such, it is not intended that the invention be limited to the particular illustrative embodiments, alternatives, and uses described above but instead that it be solely limited by the claims presented hereafter.

What is claimed is:

1. A tissue cutting device comprising:
an elongate tube having a proximal end and a distal end and a central axis extending from the proximal end to the distal end;
a first annular element at the distal end of the elongate tube;
a second annular element at the distal end of the elongate tube and concentric with the first annular element, at least one of the first or second annular elements rotatable about the central axis, the rotation causing the first annular element and the second annular element to pass each other to shear tissue.

2. The tissue device of claim 1, wherein the first annular element comprises a flat portion at its distal end perpendicular to the central axis, the flat portion extending from an outer circumference of the first annular element to the central axis; and the second annular element comprises a flat portion at its distal end perpendicular to the central axis, at least one of the first or second annular elements rotatable about the central axis, the rotation causing the first annular element and the second annular element to pass each other to shear tissue.

3. The tissue cutting device of claim 2 comprising a feature selected from the group consisting of: (1) the elongate tube has a diameter less than 5 mm; (2) at least one of the first and second annular elements has a tooth having a radial thickness of less than 50 microns; (3) the flat portion has an axial thickness of less than 100 microns; (4) the first annular element is rotatable about the central axis in an opposite direction from the second annular element; (5) the first annular element is rotatable about the central axis in a same direction as the second annular element, the first annular element and the second annular element being configured to be rotated at different speeds; (6) an intake window at the distal end of the elongate tube; (7) a hole extending along the central axis; (8) a hole extending along the central axis and an ancillary component extending through the hole, the ancillary component comprising an imaging element, a guide wire, a water jet tube, or a barbed device; (9) a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

4. The tissue cutting device of claim 1 wherein the first and second elements together form a conical shape at the distal end of the elongate tube and wherein edges of the first and second tubular elements are beveled to further shear tissue.

5. The tissue cutting device of claim 4 comprising a feature selected from the group consisting of: (1) the elongate tube has a diameter less than 5 mm; (2) the beveled edges have a thickness less than 10 microns; (3) the first annular element is rotatable about the central axis in an opposite direction from the second annular element; (4) the first and second elements together form a second conical shape, the second conical shape facing proximally; (5) the first annular element is rotatable about the central axis in a same direction as the second annular element, the first annular element and the second annular element being configured to rotate at different speeds; (6) an intake window at the distal end of the elongate tube; (7) a hole extending along the central axis; (8) a hole extending along the central axis and an ancillary component extending through the hole, the ancillary component comprising an imaging element, a guide wire, a water jet tube, or a barbed device; (9) a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

6. The tissue cutting device of claim 1 wherein the first and second annular elements each have an axially-extending cutting surface, the rotation causing the axially-extending surfaces of the first and second annular elements to pass each other to shear tissue, and wherein the first and second annular elements each have a radially-extending cutting surface, rotation causing the axially-extending surfaces of the first and second elements to pass each other to shear tissue, wherein the axially extending cutting surface has an axial length of less than 100 microns.

7. The tissue cutting device of claim 6 comprising a feature selected from the group consisting of: (1) teeth extending along the axially-extending or radially-extending cutting surfaces; (2) the elongate tube has a diameter less than 0.5 mm; (3) the first annular element is rotatable about the central axis in an opposite direction from the second annular element; (4) the first annular element is rotatable about the central axis in a same direction as the second annular element, the first annular element and the second annular element being configured to be rotated at different speeds; (5) an intake window at the distal end of the elongate tube; (6) a hole extending along the central axis; (7) a hole extending along the central axis and an ancillary component extending through the hole, the ancillary component comprising an imaging element, a guide wire, a water jet tube, or a barbed device; and (8) a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

8. The tissue cutting device of claim 1 wherein the first and second annular elements each include axially-extending teeth, the teeth having a radial thickness of less than 10 microns, the rotation causing the teeth of the first annular element and the teeth of the second annular element to pass each other to shear tissue.

9. The tissue cutting device of claim 8, comprising a feature selected from the group consisting of: (1) the elongate tube has a diameter less than 5 mm; (2) the first annular element is rotatable about the central axis in an opposite direction from the second annular element; (3) the first annular element is rotatable about the central axis in a same direction as the second annular element, the first annular element and the second annular element being configured to be rotated at different speeds; (4) the teeth have a pitch of less than 200 microns; (5) an intake window at the distal end of the elongate tube; (6) a hole extending along the central axis; (7) an ancillary component extending through the hole, the ancillary component comprising an imaging element, a guide wire, a water jet tube, or a barbed device; and (8) a third annular element and a fourth annular element, the third and fourth annular elements located between the proximal and distal ends, at least one of the third or fourth annular elements configured to rotate, the rotation causing the third and fourth annular elements to rotate past each other to further shear the tissue.

10. The tissue cutting device of claim 1 wherein the first annular element comprises a plurality of first shearing elements, each first shearing element having a perpendicular shearing surface that is perpendicular to the central axis, wherein the second annular element comprises a plurality of second shearing elements, each second shearing element having a perpendicular shearing surface that is perpendicular to the central axis, wherein the rotation causes the perpendicular shearing surfaces of the first shearing elements and the perpendicular shearing surfaces of the second shearing elements to pass each other to shear tissue.

11. The tissue cutting device of claim 10 comprising at least one feature selected from the group consisting of: (1) at least some of the perpendicular shearing surfaces of the first shearing elements lie along the same plane; (2) at least some of the perpendicular shearing surfaces are located at the same radial distance from the central axis; (3) at least some of the perpendicular shearing surfaces do not lie along the same plane; (4) at least some perpendicular shearing surfaces are located at different radial distances from the central axis; (5) each first shearing element has a parallel shearing surface that is parallel to the central axis, each second shearing element has a parallel shearing surface that is parallel to the central axis, and rotation of one or both of the first and second annular elements causes the parallel shearing surfaces of the first shearing elements and the parallel shearing surfaces of the second shearing elements to pass each other to shear tissue; (6) each first and each second shearing element has a parallel shearing surface that is parallel to the central axis, and rotation of one or both of the first and second annular elements causes the parallel shearing surfaces of the first and second shearing elements to pass each other to shear tissue wherein at least some of the parallel shearing surfaces of the first shearing elements have a configuration selected from the group consisting of (a) lying along the same radial plane, (b) spaced apart from each other circumferentially, and (c) spaced apart from each other radially; and (7) the elongate tube has a diameter of less than 5 mm.

12. The tissue cutting device of claim 1 wherein the first annular element comprises a plurality of first shearing elements, each first shearing element having a parallel shearing surface that is parallel to the central axis, wherein the second annular element including a plurality of second shearing elements, each second shearing element having a parallel shearing surface that is parallel to the central axis, and wherein the rotation causes the parallel shearing surfaces of the first shearing elements and the parallel shearing surfaces of the second shearing elements to pass each other to shear tissue.

13. The tissue cutting device of claim 12 comprising at least one feature selected from the group consisting of: (1) at least some of the parallel shearing surfaces of the first shearing elements lie along the same radial plane; (2) at least some of the parallel shearing surfaces are spaced apart from each other axially; (3) at least some of the parallel shearing surfaces are spaced apart from each other circumferentially; (4) at least some of the parallel shearing surfaces of the first shearing elements are spaced apart from each other radially; and (5) the elongate tube has a diameter of less than 5 mm.

14. A method for removing at least part of a pituitary tumor in a patient, the method comprising:
advancing a distal end of a tissue cutter through a nostril and through the sphenoid sinus of the patient to contact a cutting member of the tissue cutter with the pituitary tumor, wherein the tissue cutter includes an outer shaft configured to enter the nostril and having an outer diameter no greater than about 10 mm, which includes a distal shaft portion and a proximal shaft portion, and wherein the distal shaft portion is sharply angled relative to the proximal shaft portion;
activating the cutting member to cut tissue from the pituitary tumor by rotating an inner drive shaft located within the outer shaft; and
moving the cut pituitary tumor tissue through a channel within at least one of the shafts toward a proximal end of the tissue cutter.

15. The method of claim 14 comprising a feature selected from the group consisting of: (1) the cutting member does not extend laterally beyond the outer diameter of the tissue cutter outer shaft; (2) before contacting the pituitary tumor the method provides (a) forming an opening through the sphenoid sinus; and (b) advancing the distal end of the tissue cutter through the opening; (3) before contacting the pituitary tumor the method provides (a) forming an opening through the sphenoid sinus, and (b) advancing the distal end of the tissue cutter through the opening, and wherein the opening is formed using the tissue cutter; (4) the cutting of the tissue comprises shredding the tissue; (5) the moving of the tissue comprises urging the tissue into the channel with a cutting motion of the tissue cutter; (6) the moving of the cut tissue through the channel further comprises applying suction to the channel; (7) the moving of the cut tissue through the channel further comprises applying suction to the channel and introducing fluid, via the tissue cutter, to an area at or near the distal end of the tissue cutter, wherein the applied suction moves at least some of the fluid proximally through the channel with the cut tissue; (8) the cutting member comprises at least one moveable blade and at least one stationary blade, and wherein cutting tissue comprises rotating the at least one rotating blade past the at least one stationary blade; (9) the cutting member comprises at least two interdigitated blades, and wherein cutting tissue comprises rotating the two interdigitated blades toward one another to shear tissue therebetween; (10) the cutting member is selected from the group consisting of micro-shears, graspers and biopsy forceps; (11) the distal shaft portion is angled relative to the proximal shaft portion by at least 1 degree; (12) the distal shaft portion is angled relative to the proximal shaft portion by at least 45 degrees; (13) the distal shaft portion is angled relative to the proximal shaft portion by about 90 degrees; (14) the proximal shaft portion is curved; (15) measuring an amount of the removed tissue by filtering the removed tissue from a stream of irrigation fluid; (16) measuring an amount of the removed tissue by determining motor torque in the tissue removal device during engagement of the device with the tissue and using at least one of the determined motor torque, a time period of tissue removal or a loading condition to approximate the amount of the removed tissue; (17) monitoring a location of the tissue removal device during use, using a navigation system and at least one tracking feature on the device; (18) collecting a sample of cut tissue, using a tissue capturing feature on the device, for use as a histological sample; (19) at least partially removing a blood clot from the patient through the channel, wherein removing the blood clot includes breaking up the clot using the cutting member; (20) the tissue cutter is coupled with an image guided or robotic surgical system during performance of at least part of the method; (21) protecting tissues not intended for treatment from contacting the cutting member during use of the device; and (22) stimulating a portion of the pituitary tumor using a stimulation member at or near the distal end of the tissue removal device, and deciding whether to cut the stimulated tissue, based on an observed response from the stimulation.

16. The method of claim 14 further comprising visualizing the tissue cutting using a visualization device selected from the group consisting of: (a) a straight endoscope, (b) an angled endoscope, (c) a swing prism endoscope, (d) a side viewing endoscope, (e) a flexible endoscope, (f) a CMOS digital camera, (g) an ultrasound device, and (h) a scanning single fiber endoscope.

17. A method as in claim 16, wherein the visualization device is incorporated into the tissue removal device.

18. A method for removing a volume of tissue from a tongue in a patient to treat sleep apnea, the method comprising:
cutting tissue from the tongue using a tissue cutting device having a shaft and at least one moveable cutting member attached to the shaft at a distal end of the tissue cutting device; and
moving the cut tissue through a channel of the shaft in a direction from the distal end of the tissue cutting device toward a proximal end of the device.

19. The method of claim 18 wherein before cutting the tissue, forming an incision in the tongue, and then advancing the distal end of the tissue cutting device through the incision to cut tissue within an inner portion of the tongue.

20. The method of claim 19 comprising a feature selected from the group consisting of: (1) the incision is formed using the tissue cutting device; (2) the incision is formed in a top of the tongue; (3) the incision is formed in a bottom of the tongue; (4) the incision is formed from under the patient's chin through a bottom of the tongue, and (5) closing the incision using an energy emitting member on the tissue cutting device, wherein the energy emitting member emits energy selected from the group consisting of radiofrequency, ultrasound, microwave, heat and laser energy.

21. The method of claim 19 comprising a feature selected from the group consisting of: (1) the moveable cutting member comprises at least one moveable blade and at least one stationary blade, and wherein cutting tissue comprises rotating the at least one rotating blade past the at least one stationary blade; (2) the moveable cutting member comprises at least two interdigitated tissue cutters, and wherein cutting tissue comprises rotating the two interdigitated cutters toward one another; (3) moving the cut tissue through the channel comprises applying suction to the channel; (4) moving the cut tissue through the channel comprises applying suction to the channel and wherein moving the cut tissue through the channel further comprises introducing fluid, via the tissue cutting device, to an area at or near the distal end of the tissue cutting device, wherein the applied suction moves at least some of the fluid proximally through the channel with the cut tissue; (5) the shaft of the tissue cutting device has a diameter no greater than about 10 mm, a distal tip having a length of between about 1 mm and about 25 mm, and a bend between a proximal portion of the shaft and the distal tip forming an angle between the proximal portion and the distal tip of between about 1 degree and about 90 degrees; (6) visualizing the cutting using a visualization device selected from the group consisting of (a) a straight endoscope, (b) an angled endoscope, (c) a swing prism endoscope, (d) a side viewing endoscope, (e) a flexible endoscope, (f) a CMOS digital camera, (g) an ultrasound device, and (h) a scanning single fiber endoscope; (7) providing a visualization device that is incorporated into the tissue removal device; (8) measuring an amount of the removed tissue by filtering the removed tissue from a stream of irrigation fluid; and (9) measuring an amount of the removed tissue by determining motor torque in the tissue removal device during engagement of the device with the tissue and using at least one of the determined motor torque, a time period of tissue removal or a loading condition to approximate the amount of the removed tissue.

22. The method of claim 18 wherein the tissue cutting device comprises a mechanical tissue debrider, comprising:
a shaft having a proximal portion, a distal tip disposed at an angle relative to the proximal portion, and a channel extending from a distal end of the distal tip through at least part of the proximal portion;
at least one moveable cutting member disposed at the distal end of the distal tip;
a handle coupled with the proximal portion of the shaft;
an actuator coupled with the handle for actuating the at least one moveable cutting member; and
an energy transmission member coupled with the distal tip of the shaft for transmitting an energy to the tissue, wherein the energy is selected from the group consisting of radiofrequency, ultrasound, microwave, heat and laser energy.

23. A powered scissors device comprising:
a distal housing having a fixed cutting arm located thereon;
an elongate member coupled to the distal housing and configured to introduce the distal housing to a target tissue site of the subject, the elongate member comprising an outer tube and an inner drive tube rotatably mounted within the outer tube;
a rotatable blade rotatably mounted to the distal housing, the rotatable blade having at least one cutting element configured to cooperate with the fixed arm to shear tissue therebetween;
a crown gear located at a distal end of the inner drive tube; and
a first spur gear configured to inter-engage with the crown gear and coupled with the rotatable blade to allow the crown gear to drive the rotatable blade.

24. The device of claim 23, comprising a feature selected from the group consisting of: (1) the rotatable blade has an axis of rotation that is perpendicular to an axis of rotation of the inner drive tube; (2) the rotatable blade is partially located within a slot formed within the distal housing such that the at least one cutting element is covered by the distal housing during at least half of its rotation about an axis of rotation of the rotatable blade; (3) the rotatable blade has multiple cutting elements, each of the cutting elements having a cutting edge configured to cooperate with a cutting edge of the fixed arm to shear tissue therebetween; (4) the rotatable blade has multiple cutting elements, each of the cutting elements having a cutting edge configured to cooperate with a cutting edge of the fixed arm to shear tissue therebetween and wherein every cutting edge of the multiple cutting elements of the rotatable blade lies in a common plane; (5) the cutting element is shorter than the fixed arm; (6) the cutting element has a top side and a bottom side, is flat on the top side, and has a cutting bevel provided along the bottom side; (7) the cutting element has a cutting edge that is curved, and the fixed arm has a cutting edge that is curved in the same direction; (8) the cutting element has a cutting edge that is curved, and the fixed arm has a cutting edge that is curved in the same direction and wherein the cutting edges of the cutting element and the fixed arm are curved in an outward direction trailing away from a direction of rotation of the cutting element; (9) the cutting element has a cutting edge that is curved, and the fixed arm has a cutting edge that is curved in the same direction and wherein the cutting edge of the cutting element has a smaller radius of curvature than a radius of curvature of the cutting edge of the fixed arm; and (11) the fixed arm is provided with a radio frequency electrode
